SEQUENCE LISTING (195 sequences)

SEQ ID NO: 1 - protein, 13 aa, Homo sapiens
  TLR4 juxtamembrane domain
PVLSLNITCQMNK

SEQ ID NO: 2 - protein, 46 aa, Homo sapiens
  MRC1 signaling domain
YKKRRVHLPQEGAFENTLYFNSQSSPGTSDMKDLVGNIEQNEHSVI

SEQ ID NO: 3 - protein, 473 aa, Homo sapiens
  MERTK signaling domain
KRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 4 - protein, 476 aa, Mus musculus
  MERTK signaling domain
ALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 5 - protein, 440 aa, Homo sapiens
  Tyro3 signaling domain
LRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 6 - protein, 422 aa, Homo sapiens
  Axl signaling domain
HRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 7 - protein, 727 aa, Homo sapiens
  ELMO signaling domain
MPPPADIVKVAIEWPGAYPKLMEIDQKKPLSAIIKEVCDGWSLANHEYFALQHADSSNFYITEKNRNEIKNGTILRLTTSPAQNAQQLHERIQSSSMDAKLEALKDLASLSRDVTFAQEFINLDGISLLTQMVESGTERYQKLQKIMKPCFGDMLSFTLTAFVELMDHGIVSWDTFSVAFIKKIASFVNKSAIDISILQRSLAILESMVLNSHDLYQKVAQEITIGQLIPHLQGSDQEIQTYTIAVINALFLKAPDERRQEMANILAQKQLRSIILTHVIRAQRAINNEMAHQLYVLQVLTFNLLEDRMMTKMDPQDQAQRDIIFELRRIAFDAESEPNNSSGSMEKRKSMYTRDYKKLGFINHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 8 - protein, 522 aa, Homo sapiens
  Traf6 signaling domain - full length
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 9 - protein, 261 aa, Homo sapiens
  Syk signaling domain
TLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYY

SEQ ID NO: 10 - protein, 296 aa, Homo sapiens
  MyD88 signaling domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 11 - skipped

SEQ ID NO: 12 - protein, 42 aa, Homo sapiens
  FcepsilonR1gamma signaling domain
RLKIQVRKAAITSYEKSDGVYTGLSTRNQETYETLKHEKPPQ

SEQ ID NO: 13 - protein, 61 aa, Homo sapiens
  FcgammaR1 signaling domain
RKELKRKKKWDLEISLDSGHEKKVISSLQEDRHLEEELKCQEQKEEQLQEGVHRKEPQGAT

SEQ ID NO: 14 - protein, 77 aa, Homo sapiens
  FcgammaR2A signaling domain
CRKKRISANSTDPVKAAQFEPPGRQMIAIRKRQLEETNNDYETADGGYMTLNPRAPTDDDKNIYLTLPPNDHVNSNN

SEQ ID NO: 15 - protein, 77 aa, Homo sapiens
  FcgammaR2c signaling domain
CRKKRISANSTDPVKAAQFEPPGRQMIAIRKRQPEETNNDYETADGGYMTLNPRAPTDDDKNIYLTLPPNDHVNSNN

SEQ ID NO: 16 - protein, 25 aa, Homo sapiens
  FcgammaR3A signaling domain
KTNIRSSTRDWKDHKFKWRKDPQDK

SEQ ID NO: 17 - protein, 85 aa, Homo sapiens
  BAFF-R signaling domain
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 18 - protein, 52 aa, Homo sapiens
  DAP12 signaling domain
YFLGRLVPRGRGAAEAATRKQRITETESPYQELQGQRSDVYSDLNTQRPYYK

SEQ ID NO: 19 - protein, 86 aa, Homo sapiens
  NFAM1 signaling domain
LWNKKRMRGPGKDPTRKCPDPRSASSPKQHPSESVYTALQRRETEVYACIENEDGSSPTAKQSPLSQERPHRFEDDGELNLVYENL

SEQ ID NO: 20 - protein, 29 aa, Homo sapiens
  CD79b signaling domain (amino acids 185-229)
DSKAGMEEDHTYEGLDIDQTATYEDIVTL

SEQ ID NO: 21 - protein, 45 aa, Homo sapiens
  CD79b signaling domain (amino acids 185-213)
DSKAGMEEDHTYEGLDIDQTATYEDIVTLRTGEVKWSVGEHPGQE

SEQ ID NO: 22 - protein, 185 aa, Homo sapiens
  TLR1 signaling domain
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 23 - protein, 175 aa, Homo sapiens
  TLR2 signaling domain
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 24 - protein, 179 aa, Homo sapiens
  TLR3 signaling domain
EGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 25 - protein, 187 aa, Homo sapiens
  TLR4 signaling domain
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 26 - protein, 198 aa, Homo sapiens
  TLR5 signaling domain
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 27 - protein, 189 aa, Homo sapiens
  TLR6 signaling domain
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 28 - protein, 189 aa, Homo sapiens
  TLR7 signaling domain
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 29 - protein, 193 aa, Homo sapiens
  TLR8 signaling domain
HHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 30 - protein, 193 aa, Homo sapiens
  TLR9 signaling domain
GWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 31 - protein, 303 aa, Homo sapiens
  TRAF2 signaling domain
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 32 - protein, 274 aa, Homo sapiens
  TRAF3 signaling domain
MESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 33 - protein, 155 aa, Homo sapiens
  truncated MyD88 signaling domain without TIR domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 34 - protein, 274 aa, Homo sapiens
  truncated TRAF6 signaling domain
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 35 - protein, 29 aa, Homo sapiens
  truncated NFAM1 signaling domain
SSPKQHPSESVYTALQRRETEVYACIENE

SEQ ID NO: 36 - protein, 21 aa, Homo sapiens
  Tim1 transmembrane domain
IYAGVCISVLVLLALLGVIIA

SEQ ID NO: 37 - protein, 21 aa, Homo sapiens
  Tim4 transmembrane domain
LLMIIAPSLGFVLFALFVAFL

SEQ ID NO: 38 - protein, 21 aa, Mus musculus
  Tim4 transmembrane domain
ILIIACCVGFVLMVLLFLAFL

SEQ ID NO: 39 - protein, 21 aa, Homo sapiens
  Tim3 transmembrane domain
IYIGAGICAGLALALIFGALI

SEQ ID NO: 40 - protein, 21 aa, Homo sapiens
  FcgammaR1 transmembrane domain
VLFYLAVGIMFLVNTVLWVTI

SEQ ID NO: 41 - protein, 23 aa, Homo sapiens
  FcgammaR2A transmembrane domain
IIVAVVIATAVAAIVAAVVALIY

SEQ ID NO: 42 - protein, 23 aa, Homo sapiens
  FcgammaR2B2 transmembrane domain
SSSPMGIIVAVVTGIAVAAIVAA

SEQ ID NO: 43 - protein, 23 aa, Homo sapiens
  FcgammaR2C transmembrane domain
IIVAVVTGIAVAAIVAAVVALIY

SEQ ID NO: 44 - protein, 21 aa, Homo sapiens
  FcgammaR3A transmembrane domain
VSFCLVMVLLFAVDTGLYFSV

SEQ ID NO: 45 - protein, 21 aa, Homo sapiens
  FcepsilonR1 transmembrane domain
LCYILDAILFLYGIVLTLLYC

SEQ ID NO: 46 - protein, 19 aa, Homo sapiens
  FcalphaR1 transmembrane domain
LIRMAVAGLVLVALLAILV

SEQ ID NO: 47 - protein, 21 aa, Homo sapiens
  CD8 transmembrane domain
IYIWAPLAGTCGVLLLSLVIT

SEQ ID NO: 48 - protein, 21 aa, Homo sapiens
  MERTK transmembrane domain
FGCFCGFILIGLILYISLAIR

SEQ ID NO: 49 - protein, 21 aa, Homo sapiens
  Axl transmembrane domain
YVLLGAVVAAACVLILALFLV

SEQ ID NO: 50 - protein, 21 aa, Homo sapiens
  Tyro3 transmembrane domain
VPVVLGVLTALVTAAALALIL

SEQ ID NO: 51 - protein, 22 aa, Homo sapiens
  CD4 transmembrane domain
MALIVLGGVAGLLLFIGLGIFF

SEQ ID NO: 52 - protein, 21 aa, Homo sapiens
  DAP12 transmembrane domain
GVLAGIVMGDLVLTVLIALAV

SEQ ID NO: 53 - protein, 21 aa, Homo sapiens
  MRC1 transmembrane domain
GVVIIVILLILTGAGLAAYFF

SEQ ID NO: 54 - protein, 21 aa, Homo sapiens
  TLR1 transmembrane domain
LLIVTIVATMLVLAVTVTSLC

SEQ ID NO: 55 - protein, 21 aa, Homo sapiens
  TLR2 transmembrane domain
ALVSGMCCALFLLILLTGVLC

SEQ ID NO: 56 - protein, 21 aa, Homo sapiens
  TLR3 Transmembrane domain
FFMINTSILLIFIFIVLLIHF

SEQ ID NO: 57 - protein, 21 aa, Homo sapiens
  TLR4 transmembrane domain
TIIGVSVLSVLVVSVVAVLVY

SEQ ID NO: 58 - protein, 21 aa, Homo sapiens
  TLR5 transmembrane domain
FSLFIVCTVTLTLFLMTILTV

SEQ ID NO: 59 - protein, 21 aa, Homo sapiens
  TLR6 transmembrane domain
ALVSGMCCALFLLILLTGVLC

SEQ ID NO: 60 - protein, 21 aa, Homo sapiens
  TLR7 transmembrane domain
LILFSLSISVSLFLMVMMTAS

SEQ ID NO: 61 - protein, 21 aa, Homo sapiens
  TLR8 transmembrane domain
AVILFFFTFFITTMVMLAALA

SEQ ID NO: 62 - protein, 21 aa, Homo sapiens
  TLR9 transmembrane domain
FALSLLAVALGLGVPMLHHLC

SEQ ID NO: 63 - protein, 12 aa, Artificial Sequence
  modified IgG4 hinge
ESKYGPPCPPCP

SEQ ID NO: 64 - protein, 22 aa, Homo sapiens
  GM-CSF signal peptide
MLLLVTSLLLCELPHPAFLLIP

SEQ ID NO: 65 - protein, 24 aa, Homo sapiens
  Tim4 signal peptide
MSKEPLILWLMIEFWWLYLTPVTS

SEQ ID NO: 66 - protein, 22 aa, Mus musculus
  Tim4 signal peptide
MSKGLLLLWLVTELWWLYLTPA

SEQ ID NO: 67 - protein, 18 aa, Artificial Sequence
  T2A self-cleaving peptide
EGRGSLLTCGDVEENPGP

SEQ ID NO: 68 - protein, 23 aa, Artificial Sequence
  T2A self-cleaving peptide variant
LEGGGEGRGSLLTCGDVEENPGP

SEQ ID NO: 69 - protein, 19 aa, Artificial Sequence
  T2A self-cleaving peptide variant
EGRGSLLTCGDVEENPGPR

SEQ ID NO: 70 - protein, 19 aa, Artificial Sequence
  P2A self-cleaving peptide
ATNFSLLKQAGDVEENPGP

SEQ ID NO: 71 - protein, 27 aa, Artificial Sequence
  P2A self-cleaving peptide variant
RAKRSGSGATNFSLLKQAGDVEENPGP

SEQ ID NO: 72 - protein, 20 aa, Artificial Sequence
  E2A self-cleaving peptide
QCTNYALLKLAGDVESNPGP

SEQ ID NO: 73 - protein, 22 aa, Artificial Sequence
  F2A self-cleaving peptide
VKQTLNFDLLKLAGDVESNPGP

SEQ ID NO: 74 - skipped

SEQ ID NO: 75 - protein, 24 aa, Artificial Sequence
  T2A self-cleaving peptide variant
LEGGGEGRGSLLTCGDVEENPGPR

SEQ ID NO: 76-81 - skipped (6 entries)

SEQ ID NO: 82 - protein, 285 aa, Homo sapiens
  truncated EGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCH

SEQ ID NO: 83 - protein, 192 aa, Homo sapiens
  Rac1
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 84 - protein, 215 aa, Homo sapiens
  Rab5
MASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 85 - protein, 207 aa, Homo sapiens
  Rab7
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 86 - protein, 184 aa, Homo sapiens
  Rap1
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 87 - protein, 193 aa, Homo sapiens
  RhoA
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 88-89 - skipped (2 entries)

SEQ ID NO: 90 - protein, 598 aa, Artificial Sequence
  HPV16 E7 TCRbeta chain-P2A-TCRalpha chain
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 91 - protein, 279 aa, Mus musculus
  Tim4 binding domain, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 92 - protein, 135 aa, Artificial Sequence
  TCR Vbeta region
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVL

SEQ ID NO: 93 - protein, 173 aa, Artificial Sequence
  TCR Cbeta region, Cys-substituted
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 94 - protein, 126 aa, Artificial Sequence
  TCR Valpha region
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIP

SEQ ID NO: 95 - protein, 137 aa, Artificial Sequence
  TCR Calpha region, Cys-substituted, LVL substituted
NIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 96 - protein, 314 aa, Homo sapiens
  Tim4 binding domain, amino acids 1-24 signal peptide
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 97 - protein, 487 aa, Artificial Sequence
  CER5, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 98 - protein, 498 aa, Artificial Sequence
  CER19, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 99 - protein, 493 aa, Artificial Sequence
  CER21, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 100 - protein, 386 aa, Artificial Sequence
  CER25; amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENL

SEQ ID NO: 101 - protein, 475 aa, Artificial Sequence
  CER27, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 102 - protein, 574 aa, Artificial Sequence
  CER29, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 103 - protein, 574 aa, Artificial Sequence
  CER31, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 104 - protein, 70 aa, Homo sapiens
  FcgammaR2B2 signaling domain
VVALIYCRKKRISALPGYPECREMGETLPEKPANPTNPDEADKVGAENTITYSLLMHPDALEEPDDQNRI

SEQ ID NO: 105 - protein, 41 aa, Homo sapiens
  FcalphaR1 signaling domain
ENWHSHTALNKEASADVAEPSWSQQMCQPGLTFARTPSVCK

SEQ ID NO: 106 - protein, 141 aa, Homo sapiens
  MyD88 TIR domain
HMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 107 - protein, 27 aa, Homo sapiens
  CD28 transmembrane domain
FWVLVVVGGVLACYSLLVTVAFIIFWV

SEQ ID NO: 108 - protein, 191 aa, Homo sapiens
  CDC42
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 109 - protein, 776 aa, Artificial Sequence
  CER1, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 110 - protein, 487 aa, Artificial Sequence
  CER6, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 111 - protein, 500 aa, Artificial Sequence
  CER7, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 112 - protein, 740 aa, Artificial Sequence
  CER8, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 113 - protein, 352 aa, Artificial Sequence
  CER9, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYK

SEQ ID NO: 114 - protein, 352 aa, Artificial Sequence
  CER10, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGVLAGIVMGDLVLTVLIALAVYFLGRLVPRGRGAAEAATRKQRITETESPYQELQGQRSDVYSDLNTQRPYYK

SEQ ID NO: 115 - protein, 722 aa, Artificial Sequence
  CER11, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 116 - protein, 342 aa, Artificial Sequence
  CER12, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLRLKIQVRKAAITSYEKSDGVYTGLSTRNQETYETLKHEKPPQ

SEQ ID NO: 117 - protein, 342 aa, Artificial Sequence
  CER13, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTLCYILDAILFLYGIVLTLLYCRLKIQVRKAAITSYEKSDGVYTGLSTRNQETYETLKHEKPPQ

SEQ ID NO: 118 - protein, 455 aa, Artificial Sequence
  CER15, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 119 - protein, 441 aa, Artificial Sequence
  CER16, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 120 - protein, 479 aa, Artificial Sequence
  CER17, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 121 - protein, 479 aa, Artificial Sequence
  CER18, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 122 - protein, 498 aa, Artificial Sequence
  CER20, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 123 - protein, 493 aa, Artificial Sequence
  CER22, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 124 - protein, 493 aa, Artificial Sequence
  CER23, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 125 - protein, 493 aa, Artificial Sequence
  CER24, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 126 - protein, 485 aa, Artificial Sequence
  CER26, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 127 - protein, 489 aa, Artificial Sequence
  CER28, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 128 - protein, 603 aa, Artificial Sequence
  CER30, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 129 - protein, 540 aa, Artificial Sequence
  CER85, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 130 - protein, 507 aa, Artificial Sequence
  CER86, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGYFLGRLVPRGRGAAEAATRKQRITETESPYQELQGQRSDVYSDLNTQRPYYK

SEQ ID NO: 131 - protein, 540 aa, Artificial Sequence
  CER87, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSWRRRQRRLRGASSAEAPDGDKDAPEPLDKVIILSPGISDATAPAWPPPGEDPGTTPPGHSVPVPATELGSTELVTTKTAGPEQQMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 132 - protein, 507 aa, Artificial Sequence
  CER88, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYKMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 133 - protein, 500 aa, Artificial Sequence
  CER89, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGDSKAGMEEDHTYEGLDIDQTATYEDIVTLRTGEVKWSVGEHPGQE

SEQ ID NO: 134 - protein, 541 aa, Artificial Sequence
  CER90, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGLWNKKRMRGPGKDPTRKCPDPRSASSPKQHPSESVYTALQRRETEVYACIENEDGSSPTAKQSPLSQERPHRFEDDGELNLVYENL

SEQ ID NO: 135 - protein, 692 aa, Artificial Sequence
  CER91, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGGSGATNFSLLKQAGDVEENPGPMASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 136 - protein, 931 aa, Artificial Sequence
  CER92, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 137 - protein, 861 aa, Artificial Sequence
  CER93, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 138 - protein, 828 aa, Artificial Sequence
  CER94, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMYFLGRLVPRGRGAAEAATRKQRITETESPYQELQGQRSDVYSDLNTQRPYYK

SEQ ID NO: 139 - protein, 821 aa, Artificial Sequence
  CER95, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMDSKAGMEEDHTYEGLDIDQTATYEDIVTLRTGEVKWSVGEHPGQE

SEQ ID NO: 140 - protein, 862 aa, Artificial Sequence
  CER96, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMLWNKKRMRGPGKDPTRKCPDPRSASSPKQHPSESVYTALQRRETEVYACIENEDGSSPTAKQSPLSQERPHRFEDDGELNLVYENL

SEQ ID NO: 141 - protein, 774 aa, Artificial Sequence
  CER97, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGAYFLGRLVPRGRGAAEAATRKQRITETESPYQELQGQRSDVYSDLNTQRPYYK

SEQ ID NO: 142 - protein, 767 aa, Artificial Sequence
  CER98, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGADSKAGMEEDHTYEGLDIDQTATYEDIVTLRTGEVKWSVGEHPGQE

SEQ ID NO: 143 - protein, 808 aa, Artificial Sequence
  CER99, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGALWNKKRMRGPGKDPTRKCPDPRSASSPKQHPSESVYTALQRRETEVYACIENEDGSSPTAKQSPLSQERPHRFEDDGELNLVYENL

SEQ ID NO: 144 - protein, 579 aa, Artificial Sequence
  CER102, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYLWNKKRMRGPGKDPTRKCPDPRSASSPKQHPSESVYTALQRRETEVYACIENEDGSSPTAKQSPLSQERPHRFEDDGELNLVYENL

SEQ ID NO: 145 - protein, 538 aa, Artificial Sequence
  CER103A, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYDSKAGMEEDHTYEGLDIDQTATYEDIVTLRTGEVKWSVGEHPGQE

SEQ ID NO: 146 - protein, 522 aa, Artificial Sequence
  CER103B, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYDSKAGMEEDHTYEGLDIDQTATYEDIVTL

SEQ ID NO: 147 - protein, 545 aa, Artificial Sequence
  CER104, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYYFLGRLVPRGRGAAEAATRKQRITETESPYQELQGQRSDVYSDLNTQRPYYK

SEQ ID NO: 148 - protein, 578 aa, Artificial Sequence
  CER105, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 149 - protein, 579 aa, Artificial Sequence
  CER106, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 150 - protein, 522 aa, Artificial Sequence
  CER107, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLDSKAGMEEDHTYEGLDIDQTATYEDIVTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 151 - protein, 545 aa, Artificial Sequence
  CER108, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYKHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 152 - protein, 578 aa, Artificial Sequence
  CER109, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSWRRRQRRLRGASSAEAPDGDKDAPEPLDKVIILSPGISDATAPAWPPPGEDPGTTPPGHSVPVPATELGSTELVTTKTAGPEQQHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 153 - protein, 626 aa, Artificial Sequence
  CER110, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAYFLGRLVPRGRGAAEAATRKQRITETESPYQELQGQRSDVYSDLNTQRPYYK

SEQ ID NO: 154 - protein, 619 aa, Artificial Sequence
  CER111A, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLADSKAGMEEDHTYEGLDIDQTATYEDIVTLRTGEVKWSVGEHPGQE

SEQ ID NO: 155 - protein, 603 aa, Artificial Sequence
  CER111B, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLADSKAGMEEDHTYEGLDIDQTATYEDIVTL

SEQ ID NO: 156 - protein, 660 aa, Artificial Sequence
  CER112, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLALWNKKRMRGPGKDPTRKCPDPRSASSPKQHPSESVYTALQRRETEVYACIENEDGSSPTAKQSPLSQERPHRFEDDGELNLVYENL

SEQ ID NO: 157 - protein, 659 aa, Artificial Sequence
  CER113, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLASWRRRQRRLRGASSAEAPDGDKDAPEPLDKVIILSPGISDATAPAWPPPGEDPGTTPPGHSVPVPATELGSTELVTTKTAGPEQQ

SEQ ID NO: 158 - protein, 1050 aa, Artificial Sequence
  CER114, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 159 - protein, 1050 aa, Artificial Sequence
  CER115, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 160 - protein, 767 aa, Artificial Sequence
  CER116, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 161 - protein, 767 aa, Artificial Sequence
  CER117, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 162 - protein, 571 aa, Artificial Sequence
  CER118, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKLWNKKRMRGPGKDPTRKCPDPRSASSPKQHPSESVYTALQRRETEVYACIENEDGSSPTAKQSPLSQERPHRFEDDGELNLVYENL

SEQ ID NO: 163 - protein, 514 aa, Artificial Sequence
  CER119B, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKDSKAGMEEDHTYEGLDIDQTATYEDIVTL

SEQ ID NO: 164 - protein, 537 aa, Artificial Sequence
  CER120, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKYFLGRLVPRGRGAAEAATRKQRITETESPYQELQGQRSDVYSDLNTQRPYYK

SEQ ID NO: 165 - protein, 759 aa, Artificial Sequence
  CER121, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 166 - protein, 113 aa, Homo sapiens
  wildtype CD3zeta signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 167 - protein, 112 aa, Homo sapiens
  variant CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 42 aa, Homo sapiens
  4-1BB costimulatory signaling domain
KRGRKKLLYIFKQPFMRPVQTTQEEDGCSCRFPEEEEGGCEL

SEQ ID NO: 169 - protein, 41 aa, Homo sapiens
  CD28 costimulatory signaling domain variant, L186G/L187G substitutions with positions in reference to full length protein
RSKRSRGGHSDYMNMTPRRPGPTRKHYQPYAPPRDFAAYRS

SEQ ID NO: 170 - protein, 41 aa, Homo sapiens
  wildtype CD28 costimulatory signaling domain
RSKRSRLLHSDYMNMTPRRPGPTRKHYQPYAPPRDFAAYRS

SEQ ID NO: 171 - protein, 527 aa, Artificial Sequence
  CER122, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSYFLGRLVPRGRGAAEAATRKQRITETESPYQELQGQRSDVYSDLNTQRPYYK

SEQ ID NO: 172 - protein, 749 aa, Artificial Sequence
  CER123, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 173 - protein, 561 aa, Artificial Sequence
  CER124, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSLWNKKRMRGPGKDPTRKCPDPRSASSPKQHPSESVYTALQRRETEVYACIENEDGSSPTAKQSPLSQERPHRFEDDGELNLVYENL

SEQ ID NO: 174 - protein, 520 aa, Artificial Sequence
  CER125A, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSDSKAGMEEDHTYEGLDIDQTATYEDIVTLRTGEVKWSVGEHPGQE

SEQ ID NO: 175 - protein, 504 aa, Artificial Sequence
  CER125B, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSDSKAGMEEDHTYEGLDIDQTATYEDIVTL

SEQ ID NO: 176 - protein, 1108 aa, Artificial Sequence
  CER5_T2A_HPV16 E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSILEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 177 - protein, 1119 aa, Artificial Sequence
  CER19 _T2A_HPV16 E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATISLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 178 - protein, 1114 aa, Artificial Sequence
  CER21_T2A_HPV16 E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 179 - protein, 1007 aa, Artificial Sequence
  CER_25_T2A_HPV16 E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENLLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 180 - protein, 1096 aa, Artificial Sequence
  CER_27_T2A_HPV E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 181 - protein, 1195 aa, Artificial Sequence
  CER_29_T2A_HPV16 E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLALEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 182 - protein, 1195 aa, Artificial Sequence
  CER_31_T2A_HPV16 E7 TCR tandem expression cassette
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKVLEGGGEGRGSLLTCGDVEENPGPMAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 183 - protein, 530 aa, Artificial Sequence
  CER119A, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKDSKAGMEEDHTYEGLDIDQTATYEDIVTLRTGEVKWSVGEHPGQE

SEQ ID NO: 184 - protein, 778 aa, Artificial Sequence
  CER126, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 185 - protein, 778 aa, Artificial Sequence
  CER127, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 186 - protein, 796 aa, Artificial Sequence
  CER128, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 187 - protein, 796 aa, Artificial Sequence
  CER129, amino acids 1-22 are signal peptide
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 188 - protein, 504 aa, Artificial Sequence
  hCER21
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 189 - protein, 585 aa, Artificial Sequence
  hCER29
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 190 - protein, 556 aa, Artificial Sequence
  hCER104
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYYFLGRLVPRGRGAAEAATRKQRITETESPYQELQGQRSDVYSDLNTQRPYYK

SEQ ID NO: 191 - protein, 778 aa, Artificial Sequence
  hCER116
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 192 - protein, 778 aa, Artificial Sequence
  hCER117
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 193 - protein, 538 aa, Artificial Sequence
  hCER122
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSYFLGRLVPRGRGAAEAATRKQRITETESPYQELQGQRSDVYSDLNTQRPYYK

SEQ ID NO: 194 - protein, 760 aa, Artificial Sequence
  hCER123
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 195 - protein, 789 aa, Artificial Sequence
  hCER126
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

(19) United States
(12) Patent Application Publication
(10) Pub. No.: US 2021/0024607 A1
(43) Pub. Date: Jan. 28, 2021

(54) EXPRESSION VECTORS FOR CHIMERIC ENGULFMENT RECEPTORS, GENETICALLY MODIFIED HOST CELLS, AND USES THEREOF

(71) Applicant: CERo Therapeutics, Inc., South San Francisco, CA (US)

(72) Inventors: Daniel Mark COREY, Menlo Park, CA (US); Ingrid IBARRA, South San Francisco, CA (US)

(21) Appl. No.: 17/040,464

(22) PCT Filed: Mar. 27, 2019
(86) PCT No.: PCT/US2019/024441

§ 371 Date: Sep. 22, 2020

Related U.S. Application Data
(60) Provisional application No. 62/649,499, filed on Mar. 28, 2018.

(51) Int. Cl. C07K 14/705; C12N 15/86; C07K 14/725; C12N 5/0783; A61K 35/17; A61K 45/06

(52) U.S. Cl. CPC C07K 14/705; C12N 15/86; C07K 14/70596; C07K 14/7051; C12N 5/0638; C12N 2510/00; A61K 45/06; C12N 2740/15042; C07K 2319/02; C07K 2319/03; A61K 35/17

(57) ABSTRACT

The present disclosure relates to tandem expression cassettes encoding chimeric engulfment receptor molecules and chimeric antigen receptors/or T cell receptor binding proteins, host cells modified to include the tandem expression cassettes, and methods of making and using such receptor molecules and modified cells.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 200265_406WO_SEQUENCE_LISTING.txt. The text file is 539 KB, was created on Mar. 26, 2019, and is being submitted electronically via EFS-Web.

### BACKGROUND

The use of T cells modified with genetically engineered receptors targeted against cancer antigens has demonstrated clinical successes in hematological malignancies (e.g., CD19 specific chimeric antigen receptor therapy in leukemias). A number of clinical trials are underway for adoptive cellular immunotherapy in the treatment of solid tumors, using engineered receptors targeting CEA, GD2, mesothelin, IL13Rα, HER2, FAP, and L1CAM, to name a few. Engineered receptors include chimeric antigen receptors (CARs) and enhanced affinity T cell receptors (TCRs). However, treatment of solid tumors presents unique challenges including: trafficking to the tumor site, physical barriers to the tumor microenvironment, a stressful metabolic landscape, and immunosuppressive mechanisms (e.g., expression of immune checkpoint molecules, production of inhibitory cytokines). Efforts to augment T-cell persistence and activity in adoptive immunotherapy treatments are ongoing.

### BRIEF DESCRIPTION OF THE DRAWINGS

FIGS. 1A-G show vector maps for exemplary tandem expression cassettes. The tandem expression cassettes harbor both a human papiloma virus 16 (HPV16) E7 protein specific TCR to induce a tumor (e.g., cervical) specific cytolytic response and a phosphatidylserine specific chimeric engulfment receptor (CER) to elicit tumor specific phagocytic activity upon cytolysis-induced phosphatidylserine exposure.

FIG. 1A shows an exemplary tandem expression cassette comprising a polynucleotide encoding a chimeric engulfment receptor 5 (CER5) construct and a polynucleotide encoding a HPV16 E7 specific TCR. CER5 is positioned upstream of the HPV16 E7 specific TCR. The sequences encoding CER5 and HPV16 E7 TCR are operably linked to a EF-1α promoter and separated by a T2A peptide. CER5 comprises a Tim4 binding domain, a Tim4 transmembrane domain, and a TLR4 engulfment signaling domain. FIG. 1B shows an exemplary tandem expression cassette comprising a polynucleotide encoding a CER19 construct and a polynucleotide encoding an HPV16 E7 specific TCR. CER19 is positioned upstream of the HPV16 E7 specific TCR. The sequences encoding CER19 and HPV16 E7 TCR are operably linked to a EF-1α promoter and separated by a T2A peptide. CER19 comprises a Tim4 binding domain, a Tim4 transmembrane domain, and a TLR5 signaling domain. FIG. 1C shows an exemplary tandem expression cassette comprising a polynucleotide encoding a CER21 construct and a polynucleotide encoding an HPV16 E7 specific TCR. CER21 is positioned upstream of the HPV16 E7 specific TCR. The sequences encoding CER21 and HPV16 E7 TCR are operably linked to an EF-1α promoter and separated by a T2A peptide. CER21 comprises a Tim4 binding domain, a Tim4 transmembrane domain, and a TLR8 signaling domain. FIG. 1D shows an exemplary tandem expression cassette comprising a polynucleotide encoding CER25 construct and a polynucleotide encoding an HPV16 E7 specific TCR. CER25 is positioned upstream of the HPV16 E7 specific TCR. The sequences encoding CER25 and HPV16 E7 TCR are operably linked to a EF-1α promoter and separated by a T2A peptide. CER25 comprises a Tim4 binding domain, a Tim4 transmembrane domain, and a NFAM1 signaling domain. FIG. 1E shows an exemplary tandem expression cassette comprising a polynucleotide encoding CER27 construct and a polynucleotide encoding an HPV16 E7 specific TCR. CER27 is positioned upstream of the HPV16 E7 specific TCR. The sequences encoding CER27 and HPV E7 TCR are operably linked to a EF-1α promoter and separated by a T2A peptide. CER27 comprises a Tim4 binding domain, a Tim4 transmembrane domain, and a TLR2 signaling domain. FIG. 1F shows an exemplary tandem expression cassette comprising a polynucleotide encoding CER29 construct and a polynucleotide encoding an HPV16 E7 specific TCR. CER29 is positioned upstream of the HPV16 E7 specific TCR. The sequences encoding CER29 and HPV16 E7 TCR are operably linked to a EF-1α promoter and separated by a T2A peptide. CER29 comprises a Tim4 binding domain, a Tim4 transmembrane domain, and a Traf6 signaling domain. FIG. 1G shows an exemplary tandem expression cassette comprising a polynucleotide encoding CER31 construct and a polynucleotide encoding an HPV16 E7 specific TCR. CER31 is positioned upstream of the HPV16 E7 specific TCR. The sequences encoding CER31 and HPV16 E7 TCR are operably linked to a EF-1α promoter and separated by a T2A peptide. CER31 comprises a Tim4 binding domain, a Tim4 transmembrane domain, and a Traf3 signaling domain.

FIG. 2 shows cytotoxicity of human primary CD8+ T cells modified with tandem expression construct comprising CER21-HPV16 E7 TCR. The images show caspase 3/7 fluorescent indicator dye emitted from HPV16 E7+ head and neck squamous cell carcinoma (SCC152) cells taken at various time points (2 hrs, 4 hrs, and 6 hrs). CD8+ T cells transduced with CER21-HPV16 E7 TCR tandem expression cassette (top row) were compared to CD8+ T cells transduced with HPV16 E7 TCR alone (bottom row) in timed co-culture experiments with SCC152 cells. The tandem expression construct confers both cytolytic and phagocytic activity in the CD8+ T cells and enhances killing capacity. Effector CD8+ T cells: target SCC152 cells were incubated ata 1:1 ratio.

FIG. 3 is a line graph showing Caspase 3/7 induction over time in HPV16 E7+ SCC152 cells upon co-culture with human primary CD8+ T cells transduced with a lentiviral vector comprising a tandem cassette HPV16 E7 TCR and Chimeric Engulfment Receptor 21 (CER21) separated by a T2A sequence. The tandem expression construct encoding HPV16 E7 TCR and CER21 confers enhanced target cell killing capacity to host CD8+ T cells as compared to host CD8+ T cells comprising HPV16 E7 TCR alone. Effector CD8+ T cells were incubated with target SCC152 cells at a 1:1 ratio. Total caspase 3/7 fluorescence was quantified over time.

FIG. 4 is a bar graph showing caspase 3/7 induction in HPV16 E7+ SCC152 cells upon co-culture with CD8+ T cells transduced with a lentiviral vector comprising a tandem cassette HPV16 E7 TCR and CER separated by a T2A sequence, Mock transduced cells were used as a negative control. Labeling of SCC152 cells with the IncuCyte® caspase 3/7 red apoptosis reagent enables detection of cells undergoing apoptosis (red fluorescence). Measurements were taken over time from co-culture experiments comparing CD8+ T cells transduced with a tandem CER-HPV16 E7 TCR cassette to CD8+ T cells transduced with HPV16 E7 TCR control.

FIG. 5 shows images of caspase 3/7 fluorescent indicator dye emitted from HPV+ head and neck squamous cell carcinoma (SCC152) cells taken 6 hrs after initiation of co-culture. Co-culture with CD8+ T cells transduced with CER-HPV16 E7 TCR tandem expression cassettes was compared to co-culture with CD8+ T cells transduced with HPV16 E7 TCR alone (2^{nd} from top). CD8+ T cells transduced with CER-HPV16 E7 TCR tandem expression cassettes show higher caspase induction than control CD8+ T cells.

FIG. 6 is a bar graph showing quantification of phagocytosis after 6 hours of co-culture of CD8+ T cells transduced with HPV16 E7 TCR, CER21-HPV16 E7 TCR tandem expression cassette, CER29-HPV16 E7 TCR tandem expression cassette, or CER31-HPV16 E7 TCR tandem expression cassette. Quantification of phagocytosis was performed by the hybrid capture software in Keyence BZ-X710 imaging system wherein % phagocytosis was determined by identifying the number of red fluorescent targets (SCC152 cells) inside blue stained effector cells (cell trace violet labeled CD8+ T cells transduced with CER-HPV16 E7 TCR tandem expression cassette−# of red internalized/# of blue)×100.

FIG. 7 is a fluorescent micrograph showing CER21-HPV16 E7 TCR tandem expression cassette transduced CD8+ T cells (violet colored) engulfing SCC152 target cells (pHrodo Red labeled). A 1:1 mixture of CER21-HPV16 E7 TCR tandem expression cassette transduced CD8+ T cells to SCC152 target cells were co-cultured for 6 hours and then imaged. Arrows indicate representative images of internalization of SCC152 cells (red) within violet-colored CD8+ T cell endosomal compartments (left image=bright field overlay, right image=fluorescent overlay). CD8+ T cells engineered with the tandem cassette clearly engulf the SCC152 tumor cell line.

FIG. 8 shows that CD8+ T cells transduced with CER21-HPV16 E7 TCR tandem expression cassette engulfed target cells in a Rac1-dependent manner. Activation and membrane recruitment of the small GTP-ase Rac1 triggers phagocytosis. The left image shows engulfment of pH rodo red labeled target cells inside blue labeled CD8+ cells transduced with CER21-HPV16 E7 TCR. The Rac1 inhibitor NSC23766 (50 μM) was added to co-culture experiments (right image) and in vitro phagocytosis/engulfment quantified. Fluorescent micrographs show that inhibition of Rac1 by small molecule abolishes in vitro engulfment in CD8+ T cells transduced with CER21-HPV16 E7 TCR tandem expression cassette (right).

FIG. 9 shows that CD8+ T cells transduced with CER29-HPV16 E7 TCR tandem expression cassette engulf target cells in a Rac1-dependent manner. Activation and membrane recruitment of the small GTP-ase Rac1 triggers phagocytosis. The left image shows engulfment of pH rodo red labeled target cells inside blue labeled CD8+ cells transduced with CER29-HPV16 E7 TCR. The Rac1 inhibitor NSC23766 (50 μM) was added to co-culture experiments (right image) and in vitro phagocytosis quantified. Fluorescent micrographs show that inhibition of Rac1 by small molecule abolishes in vitro phagocytosis in CD8+ T cells transduced with CER29-HPV16 E7 TCR tandem expression cassette (right).

FIG. 10 shows that CD8+ T cells transduced with CER31-HPV16 E7 TCR tandem expression cassette engulf target cells in a Rac1-dependent manner. Activation and membrane recruitment of the small GTP-ase Rac1 triggers phagocytosis. The left image shows engulfment of pH rodo red labeled target cells inside blue labeled CD8+ cells transduced with CER31-HPV16 E7 TCR. The Rac1 inhibitor NSC23766 (50 μM) was added to co-culture experiments (right) and in vitro phagocytosis quantified. Fluorescent micrographs show that inhibition of Rac1 by small molecule inhibitor abolishes in vitro phagocytosis/engulfment in CD8+ T cells transduced with CER31-HPV16 E7 TCR tandem expression cassette (right).

FIG. 11 shows that CD8+ T cells transduced with a tandem expression cassette comprising CER21-HPV16 E7 TCR have dual cytolytic and phagocytic functionality. CER21-HPV16 E7 TCR transduced CD8+ T cells exhibit phagocytic uptake of phosphatidylserine-coated beads in vitro. Streptavidin coated latex beads were bound with biotin-phosphatidylserine and used for phagocytosis assays. After 30 minutes incubation, CER21-HPV16 E7 TCR transduced CD8+ T cells exhibited uptake of phosphatidylserine coated beads (white arrows show representative images of engulfment events).

FIG. 12 shows a high magnification image of a CER21-HPV16 E7 TCR transduced CD8+ T cell exhibiting phagocytic uptake of phosphatidylserine-coated beads in vitro.

FIG. 13 shows light micrograph of HPV16 E7 TCR transduced CD8+ T cells co-cultured with latex beads coated with phosphatidylserine at 30 minutes following incubation. CD8+ T cells transduced with HPV16 E7 TCR alone do not exhibit uptake of latex beads coated with phosphatidylserine.

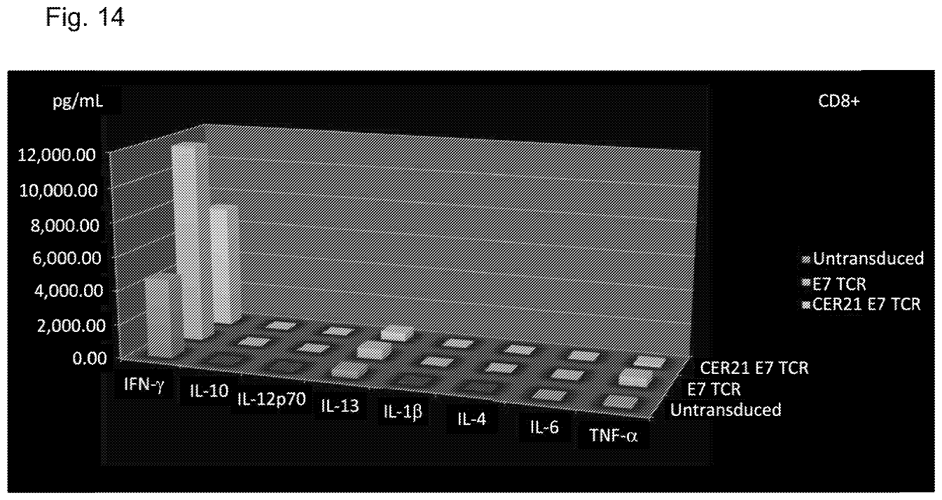

FIG. 14 is a 3D bar graph showing cytokine secretion patterns of CD8+ T cells transduced with CER21-HPV16 E7 TCR tandem expression cassette or HPV16 E7 TCR alone and co-cultured with SCC152 target cells. To determine cytokine secretion patterns, CER21-HPV16 E7 TCR modified CD8+ T cells were co-cultured with SCC152 target cells. Antigen-specific cytokine secretion was determined by measuring cytokine concentrations in the cell supernatants from each co-culture using a mesoscale multi-array cytokine plate. The following cytokines were measured in the assay: IFNγ, IL-2, TNFα, IL-4, IL-6, IL-12b, IL-13, IL-1b, and IL-10. CD8+ T cells transduced with CER21-HPV16 E7 TCR tandem expression cassette exhibit antigen specific effector function as shown by cytokine secretion, e.g., IFNγ.

FIGS. 15A-15B show that phagocytic activity of T cells is specifically induced by CER. FIG. 15A shows FACS analysis of phagocytosis assays. CellTrace-violet labeled mock transduced, HPV E7-TCR transduced T cells, and HPV E7-TCR/CER29 tandem expression cassette transduced T cells were co-cultured pHrodo-red labeled HPV+SCC152 head and neck cancer cells. FIG. 15B shows quantification of FACS data, showing no difference in phagocytosis between mock-transduced T cells and E7 TCR transduced T cells. T cells that co-expressed CER29 with the E7 TCR in T cells exhibited phagocytic activity.

### DETAILED DESCRIPTION

In one aspect, the present disclosure provides tandem expression cassettes for co-expression of a first transgene encoding a first adoptive immunotherapy molecule and a second transgene encoding a second adoptive immunotherapy molecule in the same host cell. Embodiments of the tandem expression cassettes described herein comprise a polynucleotide encoding a chimeric engulfment receptor (CER); and a polynucleotide encoding a chimeric antigen receptor (CAR) or recombinant T cell receptor (TCR). Tandem expression cassettes of the present disclosure may be used to confer tandem cytolytic and engulfment phenotypes in the same host cell. In certain embodiments, cytotoxic activity of the CAR or TCR specific for a first target antigen induces apoptosis of target cells expressing the first target antigen, thereby exposing a second target antigen; and the co-expressed CER, which is specific for the second target antigen, induces engulfment of target cells or particles expressing the second target antigen. This interaction may be related to separate genetically engineered cells producing the milieu/phosphatidylserine expression associated with the engulfment of target cells from the same cell having different interactions with a defined tumor or tumor cell.

Additionally, cells modified to express tandem expression cassettes of the present disclosure and methods and compositions for delivery of such modified cells to a subject in need thereof are provided.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means ±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Terms understood by those in the art of antibody technology are each given the meaning acquired in the art, unless expressly defined differently herein. The term “antibody” is used in the broadest sense and includes polyclonal and monoclonal antibodies. An “antibody” may refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody may be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′)₂, and Fv fragments, Fab′-SH, F(ab′)₂, diabodies, linear antibodies, scFv antibodies, VH, and multispecific antibodies formed from antibody fragments. A “Fab” (fragment antigen binding) is a portion of an antibody that binds to antigens and includes the variable region and CH1 of the heavy chain linked to the light chain via an inter-chain disulfide bond. An antibody may be of any class or subclass, including IgG and subclasses thereof (IgG₁, IgG₂, IgG₃, IgG₄), IgM, IgE, IgA, and IgD.

The term “variable region” or “variable domain” in the context of an antibody refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity determining region” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3).

As used herein, the terms “binding domain”, “binding region”, and “binding moiety” refer to a molecule, such as a peptide, oligopeptide, polypeptide, or protein that possesses the ability to specifically and non-covalently bind, associate, unite, recognize, or combine with a target molecule (e.g., tumor antigen). A binding domain includes any naturally occurring, synthetic, semi-synthetic, or recombinantly produced binding partner for a biological molecule or other target of interest. In some embodiments, the binding domain is an antigen-binding domain, such as an antibody or functional binding domain or antigen-binding portion thereof. Exemplary binding domains include single chain antibody variable regions (e.g., domain antibodies, sFv, scFv, Fab), receptor ectodomains (e.g., TNF-a), ligands (e.g., cytokines, chemokines), or synthetic polypeptides selected for the specific ability to bind to a biological molecule.

“T cell receptor” (TCR) refers to a molecule found on the surface of T cells (also referred to as T lymphocytes) that is generally responsible for recognizing antigens bound to major histocompatibility complex (MEW) molecules. The TCR is generally composed of a disulfide-linked heterodimer of the highly variable α and β chains (also known as TCRα and TCRβ, respectively) in most T cells. In a small subset of T cells, the TCR is made up of a heterodimer of γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al., Immunobiology: The Immune System in Health and Disease, 3^{rd} Ed., Current Biology Publications, p. 4:33, 1997). TCRs of the present disclosure may be from various animal species, including human, mouse, rat, cat, dog, goat, horse, or other mammals. TCRs may be cell-bound (i.e., have a transmembrane region or domain) or in soluble form. TCRs include recombinantly produced, genetically engineered, fusion, or modified forms of TCRs, including for example, scTCRs, soluble TCRs, TCR fusion constructs (TRuC™; see, U.S. Patent Publication No. 2017/0166622).

The term “variable region” or “variable domain” of a TCR α-chain (Vα) and β-chain (Vβ), or Vγ and Vδ for γδ TCRs, are involved in binding of the TCR to antigen. The V_{α} and V_{β} of a native TCR generally have similar structures, with each variable domain comprising four conserved FRs and three CDRs. The V_{α} domain is encoded by two separate DNA segments, the variable gene segment (V gene) and the joining gene segment (J gene); the V_{β} domain is encoded by three separate DNA segments, the variable gene segment (V gene), the diversity gene segment (D gene), and the joining gene segment (J gene). A single V_{α} or V_{β} domain may be sufficient to confer antigen-binding specificity.

“Major histocompatibility complex molecule” (MHC molecule) refers to a glycoprotein that delivers a peptide antigen to a cell surface. MEW class I molecules are heterodimers composed of a membrane spanning α chain (with three α domains) and a non-covalently associated β2 microglobulin. MEW class II molecules are composed of two transmembrane glycoproteins, α and β, both of which span the membrane. Each chain has two domains. MEW class I molecules deliver peptides originating in the cytosol to the cell surface, where peptide:MHC complex is recognized by CD8⁺ T cells. MEW class II molecules deliver peptides originating in the vesicular system to the cell surface, where they are recognized by CD4⁺ T cells. An MEW molecule may be from various animal species, including human, mouse, rat, or other mammals.

“Chimeric antigen receptor” (CAR) refers to a chimeric protein comprising two or more distinct domains and can function as a receptor when expressed on the surface of a cell. CARs are generally composed of an extracellular domain comprising a binding domain that binds a target antigen, an optional extracellular spacer domain, a transmembrane domain, and an intracellular signaling domain (e.g., an immunoreceptor tyrosine-based activation motif (ITAM)-containing T cell activating motif, and optionally an intracellular costimulatory domain). In certain embodiments, an intracellular signaling domain of a CAR has an ITAM-containing T cell activating domain (e.g., CD3ζ) and an intracellular costimulatory domain (e.g., CD28). In certain embodiments, a CAR is synthesized as a single polypeptide chain or is encoded by a nucleic acid molecule as a single chain polypeptide.

A variety of assays are known for identifying binding domains of the present disclosure that specifically bind a particular target, as well as determining binding domain affinities, such as Western blot, ELISA, analytical ultracentrifugation, spectroscopy, surface plasmon resonance (BIACORE®) analysis, and MEW tetramer analysis (see also, e.g., Scatchard et al., Ann. N.Y. Acad. Sci. 51:660, 1949; Wilson, Science 295:2103, 2002; Wolff et al., Cancer Res. 53:2560, 1993; Altman et al., Science 274:94-96, 1996; and U.S. Pat. Nos. 5,283,173, 5,468,614, or the equivalent). As used herein, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample.

The terms “antigen” and “Ag” refer to a molecule that is capable of inducing an immune response. The immune response that is induced may involve antibody production, the activation of specific immunologically-competent cells, or both. Macromolecules, including proteins, glycoproteins, and glycolipids, can serve as an antigen. Antigens can be derived from recombinant or genomic DNA. As contemplated herein, an antigen need not be encoded (i) solely by a full length nucleotide sequence of a gene or (ii) by a “gene” at all. An antigen can be generated or synthesized, or an antigen can be derived from a biological sample. Such a biological sample can include, but is not limited, to a tissue sample, a tumor sample, a cell, or a biological fluid.

The term “epitope” or “antigenic epitope” includes any molecule, structure, amino acid sequence or protein determinant within an antigen that is specifically bound by a cognate immune binding molecule, such as an antibody or fragment thereof (e.g., scFv), T cell receptor (TCR), CAR, chimeric engulfment receptor, or other binding molecule, domain or protein. Epitopic determinants generally contain chemically active surface groupings of molecules, such as amino acids or sugar side chains, and can have specific three dimensional structural characteristics, as well as specific charge characteristics. An epitope may be a linear epitope or a conformational epitope.

As used herein, an “effector domain” is an intracellular portion of a fusion protein or chimeric receptor that can directly or indirectly promote a biological or physiological response in a cell expressing the effector domain when receiving the appropriate signal. In certain embodiments, an effector domain is part of a protein or protein complex that receives a signal when bound. In other embodiments, the effector domain is part of a protein or protein complex that binds directly to a target molecule, which triggers a signal from the effector domain. For example, in response to binding of the CER to a target molecule, the effector domain may transduce a signal to the interior of the host cell, eliciting an effector function, e.g., engulfment, phagolysosome maturation, or secretion of anti-inflammatory, and/or immunosuppressive cytokines. An effector domain may directly promote a cellular response when it contains one or more signaling domains or motifs. In other embodiments, an effector domain will indirectly promote a cellular response by associating with one or more other proteins that directly promote a cellular response.

An “engulfment signaling domain” refers to an intracellular effector domain, which, upon binding of the target molecule (e.g., phosphatidylserine) targeted by the extracellular domain of a CER expressed by a host cell, activates one or more signaling pathways in the host cell resulting in engulfment, including, in specific embodiments, cytoskeletal rearrangement of the host cell and internalization of the target cell or particle associated with the target antigen. In certain embodiments, an engulfment signaling domain activates one or more signaling pathways resulting in phagocytosis of the target cell or particle. In further embodiments, an engulfment signaling domain comprises a primary engulfment signaling domain and a secondary engulfment signaling domain.

“Junction amino acids” or “junction amino acid residues” refer to one or more (e.g., about 2-20) amino acid residues between two adjacent motifs, regions or domains of a polypeptide. Junction amino acids may result from the construct design of a chimeric protein (e.g., amino acid residues resulting from the use of a restriction enzyme site during the construction of a nucleic acid molecule encoding a fusion protein).

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

“Nucleic acid molecule” and “polynucleotide” can be in the form of RNA or DNA, which includes cDNA, genomic DNA, and synthetic DNA. A nucleic acid molecule may be composed of naturally occurring nucleotides (such as deoxyribonucleotides and ribonucleotides), analogs of naturally occurring nucleotides (e.g., a-enantiomeric forms of naturally occurring nucleotides), or a combination of both. Modified nucleotides can have “modifications in or replacement of” sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Encoding” refers to the inherent property of specific polynucleotide sequences, such as DNA, cDNA, and mRNA sequences, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a polynucleotide encodes a protein if transcription and translation of mRNA corresponding to that polynucleotide produces the protein in a cell or other biological system. Both a coding strand and a non-coding strand can be referred to as encoding a protein or other product of the polynucleotide. Unless otherwise specified, a “nucleotide sequence encoding an amino acid sequence” includes all nucleotide sequences that are degenerate versions of each other and that encode the same amino acid sequence.

As used herein, the term “endogenous” or “native” refers to a gene, protein, compound, molecule or activity that is normally present in a host or host cell, including naturally occurring variants of the gene, protein, compound, molecule, or activity.

As used herein, “homologous” or “homolog” refers to a molecule or activity from a host cell that is related by ancestry to a second gene or activity, e.g., from the same host cell, from a different host cell, from a different organism, from a different strain, from a different species. For example, a heterologous molecule or heterologous gene encoding the molecule may be homologous to a native host cell molecule or gene that encodes the molecule, respectively, and may optionally have an altered structure, sequence, expression level or any combination thereof.

As used herein, “heterologous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous nucleic acid molecule, construct or sequence can be from a different genus or species. In some embodiments, the heterologous nucleic acid molecules are not naturally occurring. In certain embodiments, a heterologous nucleic acid molecule is added (i.e., not endogenous or native) into a host cell or host genome by, for example, conjugation, transformation, transfection, transduction, electroporation, or the like, wherein the added molecule can integrate into the host cell genome or exist as extra-chromosomal genetic material (e.g., as a plasmid or other form of self-replicating vector), and can be present in multiple copies. In addition, “heterologous” refers to a non-native enzyme, protein or other activity encoded by a non-endogenous nucleic acid molecule introduced into the host cell, even if the host cell encodes a homologous protein or activity.

As used herein, the term “engineered,” “recombinant,” “modified” or “non-natural” refers to an organism, microorganism, cell, nucleic acid molecule, or vector that has been modified by introduction of a heterologous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins, chimeric receptors, or enzymes, or other nucleic acid molecule additions, deletions, substitutions, or other functional disruption of or addition to a cell's genetic material. Exemplary modifications include those in coding regions or functional fragments thereof heterologous or homologous polypeptides from a reference or parent molecule. Additional exemplary modifications include, for example, modifications in non-coding regulatory regions in which the modifications alter expression of a gene or operon.

As used here, the term “transgene” refers to a gene or polynucleotide encoding a protein of interest (e.g., CER, CAR, TCR) whose expression is desired in a host cell and that has been transferred by genetic engineering techniques into a cell. A transgene may encode proteins of therapeutic interest as well as proteins that are reporters, tags, markers, suicide proteins, etc. A transgene may be from a natural source, modification of a natural gene, or a recombinant or synthetic molecule. In certain embodiments, a transgene is a component of a vector.

The term “overexpressed” or “overexpression” of an antigen refers to an abnormally high level of antigen expression in a cell. Overexpressed antigen or overexpression of antigen is often associated with a disease state, such as in hematological malignancies and cells forming a solid tumor within a specific tissue or organ of a subject. Solid tumors or hematological malignancies characterized by overexpression of a tumor antigen can be determined by standard assays known in the art.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “mature polypeptide” or “mature protein” refers to a protein or polypeptide that is secreted or localized in the cell membrane or inside certain cell organelles (e.g., the endoplasmic reticulum, golgi, or endosome) and does not include an N-terminal signal peptide.

A “signal peptide”, also referred to as “signal sequence”, “leader sequence”, “leader peptide”, “localization signal” or “localization sequence”, is a short peptide (usually 15-30 amino acids in length) present at the N-terminus of newly synthesized proteins that are destined for the secretory pathway. A signal peptide typically comprises a short stretch of hydrophilic, positively charged amino acids at the N-terminus, a central hydrophobic domain of 5-15 residues, and a C-terminal region with a cleavage site for a signal peptidase. In eukaryotes, a signal peptide prompts translocation of the newly synthesized protein to the endoplasmic reticulum where it is cleaved by the signal peptidase, creating a mature protein that then proceeds to its appropriate destination.

The “percent identity” between two or more nucleic acid or amino acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:N.Y. (1975), pp. 71-77; Lewin, Genes IV, Oxford University Press, NY and Cell Press, Cambridge, Mass. (1990), p. 8).

The term “chimeric” refers to any nucleic acid molecule or protein that is not endogenous and comprises a combination of sequences joined or linked together that are not naturally found joined or linked together in nature. For example, a chimeric nucleic acid molecule may comprise nucleic acids encoding various domains from multiple different genes. In another example, a chimeric nucleic acid molecule may comprise regulatory sequences and coding sequences that are derived from different sources, or regulatory sequences and coding sequences that are derived from the same source but arranged in a manner different than that found in nature.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that a promoter is in the correct location and orientation in relation to a polynucleotide to control the initiation of transcription by RNA polymerase and expression of the polynucleotide.

A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment.

In certain embodiments, the vector is a viral vector. Examples of viral vectors include, but are not limited to, adenovirus vectors, adeno-associated virus vectors, retrovirus vectors, gammaretrovirus vectors, and lentivirus vectors. “Retroviruses” are viruses having an RNA genome. “Gammaretrovirus” refers to a genus of the retroviridae family. Examples of gammaretroviruses include mouse stern cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HTV type 1 and HIV type 2, equine infectious anemia virus, feline immunodeficiency virus (FIV), bovine immune deficiency virus (BIV), and simian immunodeficiency virus (SIV).

In other embodiments, the vector is a non-viral vector. Examples of non-viral vectors include lipid-based DNA vectors, modified mRNA (modRNA), self-amplifying mRNA, closed-ended linear duplex (MA)) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

As used herein, the term “expression cassette” refers to a distinct component of a vector nucleic acid comprising at least one transgene and regulatory sequences controlling its expression (e.g., promoter, 3′UTR) in a host cell. A tandem expression cassette refers to a component of a vector nucleic acid comprising at least two transgenes under the control of the same set of regulatory sequences for tandem expression of the at least two transgenes. In certain embodiments, the tandem expression cassette comprises at least two transgenes under the control of the same promoter. In certain embodiments, the first transgene and second transgene are separated by an internal ribosome entry site (TRES), furin cleavage site, or self-cleaving viral 2A peptide to allow for co-expression of two proteins from a single mRNA.

A “particle” refers to a fragment of a cell or a small object of at least 10 nm and up to 50 μm in diameter. A particle may be derived from a living cell or organism, the environment, or synthetic. A particle can be a viral particle, prion particle, protein particle, synthetic particle, small mineral particle, or cellular debris.

As used herein, the term “engulfment” refers to a receptor-mediated process wherein endogenous or exogenous cells or particles greater than 10 nm in diameter are internalized by a phagocyte or host cell of the present disclosure. Engulfment is typically composed of multiple steps: (1) tethering of the target cell or particle via binding of an engulfment receptor to a pro-engulfment marker or antigenic marker directly or indirectly (via a bridging molecule) on a target cell or particle; and (2) internalization or engulfment of the whole target cell or particle, or a portion thereof. In certain embodiments, internalization may occur via cytoskeletal rearrangement of a phagocyte or host cell to form a phagosome, a membrane-bound compartment containing the internalized target. Engulfment may further include maturation of the phagosome, wherein the phagosome becomes increasingly acidic and fuses with lysosomes (to form a phagolysosome), whereupon the engulfed target is degraded (e.g., “phagocytosis”). Alternatively, phagosome-lysosome fusion may not be observed in engulfment. In yet another embodiment, a phagosome may regurgitate or discharge its contents to the extracellular environment before complete degradation. In some embodiments, engulfment refers to phagocytosis. In some embodiments, engulfment includes tethering of the target cell or particle by the phagocyte of host cell of the present disclosure, but not internalization. In some embodiments, engulfment includes tethering of the target cell or particle by the phagocyte of host cell of the present disclosure and internalization of part of the target cell or particle.

As used herein, the term “phagocytosis” refers to an engulfment process of cells or large particles (≥0.5 μm) wherein tethering of a target cell or particle, engulfment of the target cell or particle, and degradation of the internalized target cell or particle occurs. In certain embodiments, phagocytosis comprises formation of a phagosome that encompasses the internalized target cell or particle and phagosome fusion with a lysosome to form a phagolysosome, wherein the contents therein are degraded. In certain embodiments, following binding of a CER expressed on a host cell of the present disclosure to a target antigen expressed by a target cell or particle, a phagocytic synapse is formed; an actin-rich phagocytic cup is generated at the phagocytic synapse; phagocytic arms are extended around the target cell or particle through cytoskeletal rearrangements; and ultimately, the target cell or particle is pulled into the phagocyte or host cell through force generated by motor proteins. As used herein, “phagocytosis” includes the process of “efferocytosis”, which specifically refers to the phagocytosis of apoptotic or necrotic cells in a non-inflammatory manner.

The term “immune system cell” or “immune cell” means any cell of the immune system that originates from a hematopoietic stem cell in the bone marrow. Hematopoietic stem cells give rise to two major lineages: myeloid progenitor cells (which give rise to myeloid cells such as monocytes, macrophages, dendritic cells, megakaryocytes and granulocytes) and lymphoid progenitor cells (which give rise to lymphoid cells such as T cells, B cells and natural killer (NK) cells). Exemplary immune system cells include a CD4+ T cell, a CD8+ T cell, a CD4- CD8-double negative T cell, a γδ T cell, a regulatory T cell, a natural killer cell, and a dendritic cell. Macrophages and dendritic cells may also be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

The term “T cells” refers to cells of T cell lineage. “Cells of T cell lineage” refer to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g. , CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naïve T cells (CD45 RA+, CCR7+, CD62L+, CD27+, CD45RO−), central memory T cells (CD45RO⁺, CD62L⁺, CD8⁺), effector memory T cells (CD45RA+, CD45RO−, CCR7−, CD62L−, CD27−), mucosal-associated invariant T (MAIT) cells, Tregs, natural killer T cells, and tissue resident T cells.

The term “B cells” refers to cells of the B cell lineage. “Cells of B cell lineage” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g. , CD19⁺, CD72+, CD24+, CD20⁺), or a physiological, morphological, functional, or immunological feature specific for a B cell. For example, cells of the B cell lineage may be progenitor or precursor cells committed to the B cell lineage (e.g., pre-pro-B cells, pro-B cells, and pre-B cells); immature and inactivated B cells or mature and functional or activated B cells. Thus, “B cells” encompass naïve B cells, plasma cells, regulatory B cells, marginal zone B cells, follicular B cells, lymphoplasmacytoid cells, plasmablast cells, and memory B cells (e.g., CD27⁺, IgD⁻).

The term “cytotoxic activity,” also referred to as “cytolytic activity,” with respect to a cell (e.g., T cell) that expresses an immune receptor (e.g., TCR) on its surface, means that upon antigen-specific signaling (e.g., via the TCR) the cell induces a target cell to undergo apoptosis. In some embodiments, a cytotoxic cell may induce apoptosis in a target cell via the release of cytotoxins, such as perforin, granzyme, and granulysin, from granules. Perforins insert into the target cell membrane and form pores that allow water and salts to rapidly enter the target cell. Granzymes are serine proteases that induce apoptosis in the target cell. Granulysin is also capable of forming pores in the target cell membrane and is a proinflammatory molecule. In some embodiments, a cytotoxic cell may induce apoptosis in a target cell via interaction of Fas ligand, which is upregulated on T cell following antigen-specific signaling, with Fas molecules expressed on the target cell. Fas is an apoptosis-signaling receptor molecule on the surface of a number of different cells.

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

The term “cancer” as used herein is defined as disease characterized by the rapid and uncontrolled growth of aberrant cells. The aberrant cells may form solid tumors or constitute a hematological malignancy. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “subject,” “patient” and “individual” are used interchangeably herein and are intended to include living organisms in which an immune response can be elicited (e.g., mammals). Examples of subjects include humans, primates, cows, horses, goats, sheep, dogs, cats, mice, rats, rabbits, guinea pigs, pigs, and transgenic species thereof.

“Adoptive cellular immunotherapy” or “adoptive immunotherapy” refers to the administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

“Autologous” refers to a graft (e.g., organ, tissue, cells)derived from the same subject to which it is later to be re-introduced.

“Allogeneic” refers to a graft derived from a different subject of the same species.

A “therapeutically effective amount” or “effective amount” of a chimeric protein or cell expressing a chimeric protein of this disclosure (e.g., a tandem expression cassette or a cell expressing a tandem expression cassette) refers to that amount of protein or cells sufficient to result in amelioration of one or more symptoms of the disease, disorder, or undesired condition being treated. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously.

“Treat” or “treatment” or “ameliorate” refers to medical management of a disease, disorder, or undesired condition of a subject. In general, an appropriate dose or treatment regimen comprising a host cell expressing a chimeric protein of this disclosure is administered in an amount sufficient to elicit a therapeutic or prophylactic benefit. Therapeutic or prophylactic/preventive benefit includes improved clinical outcome; lessening or alleviation of symptoms associated with a disease, disorder, or undesired condition; decreased occurrence of symptoms; improved quality of life; longer disease-free status; diminishment of extent of disease, disorder, or undesired condition; stabilization of disease state; delay of disease progression; remission; survival; prolonged survival; or any combination thereof.

The term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with a cancerous condition. An “anti-tumor effect” can also be manifested by prevention of a hematological malignancy or tumor formation.

Additional definitions are provided throughout the present disclosure.

### Transgenes

Tandem expression cassettes of the present disclosure comprise at least two transgenes encoding a chimeric engulfment receptor (CER) and a chimeric antigen receptor (CAR)/or a T cell receptor (TCR). Certain embodiments of the tandem expression cassettes provided herein comprise: (a) a polynucleotide encoding a CER comprising: an extracellular domain comprising a binding domain that binds to a target antigen; an engulfment signaling domain; and a transmembrane domain positioned between and connecting the extracellular domain and the engulfment signaling domain; and (b) a polynucleotide encoding a CAR comprising an extracellular domain comprising a binding domain that binds to a target antigen; an intracellular signaling domain; and a transmembrane domain positioned between and connecting the extracellular domain and the intracellular signaling domain. Other embodiments of the tandem expression cassettes provided herein comprise: (a) a polynucleotide encoding a CER comprising: an extracellular domain comprising a binding domain that binds to a target antigen; an engulfment signaling domain; and a transmembrane domain positioned between and connecting the extracellular domain and the engulfment signaling domain; and (b) a polynucleotide encoding a recombinant TCR binding protein. In certain embodiments, the polynucleotide encoding the CER is positioned 5′ to the polynucleotide encoding the CAR or TCR. In other embodiments, the polynucleotide encoding the CER is positioned 3′ to the polynucleotide encoding the CAR or TCR.

Further aspects of the transgenes and their encoded cellular immunotherapy molecules are provided as follows.

### I. Chimeric Engulfment Receptors (CERs)

Tandem expression cassettes of the present disclosure comprise at least one polynucleotide encoding a CER. Chimeric engulfment receptors generally comprise: (a) an extracellular domain comprising a binding domain that binds to a target antigen, (b) an engulfment signaling domain; and (c) a transmembrane domain positioned between and connecting the extracellular domain and the engulfment signaling domain. In certain embodiments, the extracellular domain of the chimeric engulfment receptors described herein optionally includes an extracellular spacer domain positioned between and connecting the binding domain and transmembrane domain.

Chimeric engulfment receptors described herein are capable of conferring an engulfment phenotype that is specific for a target antigen to a host cell that is modified to express said chimeric engulfment receptor. In certain embodiments, expression of a CER as described herein confers an engulfment phenotype to a host cell that does not naturally exhibit an engulfment phenotype. In certain embodiments, the engulfment activity is phagocytic activity. CERs of the present disclosure may be used to redirect engulfment specificity to target cells that express the targeted antigen.

### Extracellular Domains

As described herein, a CER comprises an extracellular domain specific to a target antigen. In certain embodiments, the extracellular domain comprises a binding domain that specifically binds a target antigen (e.g., phosphatidylserine). Binding of a target molecule by the binding domain may block the interaction between the target molecule (e.g., a receptor or a ligand) and another molecule and, for example, interfere with, reduce or eliminate certain functions of the target molecule (e.g., signal transduction). In some embodiments, the binding of a target molecule may induce certain biological pathways or identify the target molecule or cell expressing the target molecule for elimination.

A binding domain suitable for use in a CER of the present disclosure may be any polypeptide or peptide that specifically binds a target molecule of interest, e.g., phosphatidylserine. Sources of binding domains include extracellular domains of receptors, ligands for cell surface receptors or molecules, and antibodies or antigen binding portions, such as antibody variable regions from various species. For example a binding domain may comprise a, sFv, scFv, Fab, scFv-based grababody, VH domain, VL domain, single domain camelid antibody (VHH), or domain antibody. A binding domain may be derived from a human, primate, rodent, avian, or ovine. Additional sources of binding domains include variable regions of antibodies from other species, such as camelid (from camels, dromedaries, or llamas; Ghahroudi et al., FEBS Lett. 414:521, 1997; Vincke et al., J. Biol. Chem. 284:3273, 2009; Hamers-Casterman et al., Nature 363:446, 1993 and Nguyen et al., J. Mol. Biol. 275:413, 1998), nurse sharks (Roux et al., Proc. Nat'l. Acad. Sci. (USA) 95:11804, 1998), spotted ratfish (Nguyen et al., Immunogen. 54:39, 2002), or lamprey (Herrin et al., Proc. Nat'l. Acad. Sci. (USA) 105:2040, 2008 and Alder et al. Nat. Immunol. 9:319, 2008). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008). In certain embodiments, a binding domain is murine, chimeric, human, or humanized.

In certain embodiments, the CER binding domain comprises an antibody or antigen binding fragment thereof, such as a single chain Fv fragment (scFv) that comprises VH and VL regions, specific for a target disease antigen. In certain embodiments, the antibody or antigen binding fragment is chimeric, human, or humanized. In further embodiments, the V_{H} and V_{L} regions are human or humanized.

A target molecule that is bound by an extracellular domain of a CER of the present disclosure, may be found on or in association with a cell of interest (“target cell”). Exemplary target cells include a cancer cell, a cell associated with an autoimmune disease or disorder or with an inflammatory disease or disorder, an infectious microbe (e.g., bacteria, virus, or fungi), and an infected cell (e.g., virus-infected cell). A cell of an infectious organism, such as a mammalian parasite, is also contemplated as a target cell.

In certain embodiments, the extracellular domain binds to a pro-engulfment marker. As used herein, a pro-engulfment marker is a moiety (e.g., protein, lipid, or polysaccharide) that an apoptotic, necrotic, pyroptotic, or infected cell exhibits on its surface that distinguishes it from a non-apoptotic, non-necrotic, non-pyroptotic, oncotic, or uninfected cell, respectively. A pro-engulfment marker can be an intracellular moiety that is surface exposed on an apoptotic or necrotic cell, a moiety that has altered glycosylation or altered surface charge on an apoptotic or necrotic cell, or a serum moiety that is bound to an apoptotic, necrotic, pyroptotic, or oncotic cell. Examples of pro-engulfment markers for apoptotic cells include phosphatidylserine (PtdSer), ICAM-3, oxidized low density lipoprotein, calreticulin, annexin I, complement C1q, and thrombospondin. Necrotic, oncotic, and pyroptotic cells also expose PtdSer pro-engulfment markers on the cell surface. Engulfment receptors can detect (or bind) a pro-engulfment marker on a target cell (e.g., a damaged, infected, apoptotic, necrotic, pyroptotic, or oncotic cell) directly or indirectly using soluble bridging molecules as intermediaries that bind to the pro-engulfment marker. In certain such embodiments, the pro-engulfment marker targeted by the extracellular domain is phosphatidylserine (PtdSer), ICAM-3, oxidized low density lipoprotein, calreticulin, annexin I, complement C1q, or thrombospondin. Exemplary Tim4 binding domains that bind to phosphatidylserine include amino acid sequences comprising SEQ ID NO:91, SEQ ID NO:96, amino acids 23-279 of SEQ ID NO:91, or amino acids 25-314 of SEQ ID NO:96.

In certain embodiments, the extracellular domain binds to a tumor antigen. Exemplary tumor antigens include CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor a, CD97, CD171, CD179a, CD44v6, WT1, VEGF-α, VEGFR1, IL-13Rα1, IL-13Rα2, IL-11Ra, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGRS, SSX2, AKAP-4, FLT3, fucosyl GM1, GM3, o-acetyl-GD2, and GD2.

In certain embodiments, the extracellular domain binds to a viral antigen, bacterial antigen, fungal antigen, protozoan antigen, or parasitic antigen.

In certain embodiments, the extracellular domain optionally comprises an extracellular, non-signaling spacer or linker domain. Where included, such a spacer or linker domain may position the binding domain away from the host cell surface to further enable proper cell to cell contact, binding, and activation. An extracellular spacer domain is generally located between the extracellular binding domain and the transmembrane domain of the CER. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG₄ hinge region having an amino acid sequence of ESKYGPPCPPCP (SEQ ID NO:63).

Other examples of hinge regions that may be used in the CERs described herein include the hinge region from the extracellular regions of type 1 membrane proteins, such as CD8a, CD4, CD28 and CD7, which may be wild-type or variants thereof. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D. In yet further embodiments, an extracellular spacer domain may be derived from a toll-like receptor (TLR) juxtamembrane domain. A TLR juxtamembrane domain comprises acidic amino acids lying between the leucine rich repeats (LRRs) and the transmembrane domain of a TLR. In certain embodiments, a TLR juxtamembrane domain is a TLR1, TLR2, TLR3, TLR4, TLR5, TLR6, TLR7, TLR8, or TLR9 juxtamembrane domain. An exemplary TLR juxtamembrane domain is a TLR4 juxtamembrane domain comprising an amino acid sequence of SEQ ID NO:1.

Extraceliular domains may be derived from any mammalian species, including humans, primates, cows, horses, goats, sheep, dogs, cats, mice, rats, rabbits, guinea pigs, pigs, and transgenic species thereof. In certain embodiments, an extracellular domain is murine, chimeric, human, or humanized.

### Engulfment Signaling Domains

The engulfment signaling domain of a CER is an intracellular effector domain and is capable of transmitting functional signals to a cell in response to binding of the extracellular domain of the CER to a target molecule. The engulfment signaling domain may be any portion of an engulfment signaling molecule that retains sufficient signaling activity. In some embodiments, a full length or full length intracellular component of an engulfment signaling molecule is used. In some embodiments, a truncated portion of an engulfment signaling molecule or intracellular component of an engulfment signaling molecule is used, provided that the truncated portion retains sufficient signal transduction activity. In further embodiments, an engulfment signaling domain is a variant of an entire or truncated portion of an engulfment signaling molecule, provided that the variant retains sufficient signal transduction activity (i.e., is a functional variant).

Exemplary engulfment signaling domains that may be used in a CER include a MRC1 signaling domain, a MERTK signaling domain, a Tyro3 signaling domain, an Ax1 signaling domain, an ELMO signaling domain, a Traf6 signaling domain, a Syk signaling domain, a MyD88 signaling domain, a PI3K signaling domain, a FcR signaling domain (e.g., FcγR1, FcγR2A, FcγR2C, FcγR2B2 , FcγR3A , FcγR2C , FcγR3A , FcεR1, or FcαR1 signaling domain), a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, a CD79b signaling domain, a TLR signaling domain (e.g., TLR1, TLR2, TLR3, TLR4, TLR5, TLR6, TLR7, TLR8, or TLR9 signaling domain), a Traf2 signaling domain, or a Traf3 signaling domain.

In certain embodiments, the engulfment signaling domain comprises a sequence that has at least about 75%, 80%, 85%, 90%, 91%, 92%, 93%, 94%, 95%, 96%, 97%, 98%, 99%, 99.5%, or 100% identity to a MRC1 signaling domain comprising an amino acid sequence of SEQ ID NO:2, a MERTK signaling domain comprising an amino acid sequence of SEQ ID NO:3, a Tyro3 signaling domain comprising an amino acid sequence of SEQ ID NO:5, an Ax1 signaling domain comprising an amino acid sequence of SEQ ID NO:6, an ELMO signaling domain comprising an amino acid sequence of SEQ ID NO:7, a Traf6 signaling domain comprising an amino acid sequence of SEQ ID NO:8, a Syk signaling domain comprising an amino acid sequence of SEQ ID NO:9, a MyD88 signaling domain comprising an amino acid sequence of SEQ ID NO:10, a PI3K signaling domain comprising an amino acid sequence of SEQ ID NO:11, a FcγRIγ signaling domain comprising an amino acid sequence of SEQ ID NO:12, a FcγR1 signaling domain comprising an amino acid sequence of SEQ ID NO:13, a FcγR2A signaling domain comprising an amino acid sequence of SEQ ID NO:14, a FcγR2C signaling domain comprising an amino acid sequence of SEQ ID NO:15, a FcγR3A signaling domain comprising an amino acid sequence of SEQ ID NO:16, a BAFF-R signaling domain comprising an amino acid sequence of SEQ ID NO:17, a DAP12 signaling domain comprising an amino acid sequence of SEQ ID NO:18, a NFAM1 signaling domain comprising an amino acid sequence of SEQ ID NO:19, a CD79b signaling domain comprising an amino acid sequence of SEQ ID NO:21, a TLR1 signaling domain comprising an amino acid sequence of SEQ ID NO:22, a TLR2 signaling domain comprising an amino acid sequence of SEQ ID NO:23, a TLR3 signaling domain comprising an amino acid sequence of SEQ ID NO:24, a TLR4 signaling domain comprising an amino acid sequence of SEQ ID NO:25, a TLR5 signaling domain comprising an amino acid sequence of SEQ ID NO:26, a TLR6 signaling domain comprising an amino acid sequence of SEQ ID NO:27, a TLR7 signaling domain comprising an amino acid sequence of SEQ ID NO:28, a TLR8 signaling domain comprising an amino acid sequence of SEQ ID NO:29, a TLR9 signaling domain comprising an amino acid sequence of SEQ ID NO:30, a Traf2 signaling domain comprising an amino acid sequence of SEQ ID NO:31, or a Traf3 signaling domain comprising an amino acid sequence of SEQ ID NO:32.

In some embodiments, the engulfment signaling domain is an MRC1 signaling domain comprising or consisting of an amino acid sequence of SEQ ID NO:2, a MERTK signaling domain comprising or consisting of an amino acid sequence of SEQ ID NO:3, a Tyro3 signaling domain comprising or consisting of an amino acid sequence of SEQ ID NO:5, an Ax1 signaling domain comprising or consisting of an amino acid sequence of SEQ ID NO:6, or an ELMO signaling domain comprising or consisting of an amino acid sequence of SEQ ID NO:7, a Traf6 signaling domain comprising or consisting of an amino acid sequence of SEQ ID NO:8, a Syk signaling domain comprising or consisting of an amino acid sequence of SEQ ID NO:9, a MyD88 signaling domain comprising or consisting of an amino acid sequence of SEQ ID NO:10, a PI3K signaling domain comprising or consisting of an amino acid sequence of SEQ ID NO:11, a FcεRIy signaling domain comprising or consisting of an amino acid sequence of SEQ ID NO:12, a FcγR1 signaling domain comprising or consisting of an amino acid sequence of SEQ ID NO:13, a FcγR2A signaling domain comprising or consisting of an amino acid sequence of SEQ ID NO:14, a FcγR2C signaling domain comprising or consisting of an amino acid sequence of SEQ ID NO:15, a FcγR3A signaling domain comprising or consisting of an amino acid sequence of SEQ ID NO:16, a BAFF-R signaling domain comprising or consisting of an amino acid sequence of SEQ ID NO:17, a DAP-12 signaling domain comprising or consisting of an amino acid sequence of SEQ ID NO:18, a NFAM1 signaling domain comprising or consisting of an amino acid sequence of SEQ ID NO:19, a CD79b signaling domain comprising or consisting of an amino acid sequence of SEQ ID NO:21, a TLR1 signaling domain comprising or consisting of an amino acid sequence of SEQ ID NO:22, a TLR2 signaling domain comprising or consisting of an amino acid sequence of SEQ ID NO:23, a TLR3 signaling domain comprising or consisting of an amino acid sequence of SEQ ID NO:24, a TLR4 signaling domain comprising or consisting of an amino acid sequence of SEQ ID NO:25, a TLR5 signaling domain comprising or consisting of an amino acid sequence of SEQ ID NO:26, a TLR6, signaling domain comprising or consisting of an amino acid sequence of SEQ ID NO:27, a TLR7 signaling domain comprising or consisting of an amino acid sequence of SEQ ID NO:28, a TLR8, signaling domain comprising or consisting of an amino acid sequence of SEQ ID NO:29, a TLR9 signaling domain comprising or consisting of an amino acid sequence of SEQ ID NO:30, a Traf2 signaling domain comprising or consisting of an amino acid sequence of SEQ ID NO:31, or a Traf3 signaling domain comprising or consisting of an amino acid sequence of SEQ ID NO:32.

A truncated engulfment signaling domain may be truncated at its N-terminus, its C-terminus, at both the N-terminus and C-terminus. In certain embodiments, the MRC1 engulfment signaling domain is truncated 1, 2, 3, 4, 5, or more amino acids at its N-terminus corresponding to the amino acid sequence of SEQ ID NO:2; the MERTK engulfment signaling domain is truncated 1, 2, 3, 4, 5, or more amino acids at its N-terminus corresponding to the amino acid sequence of SEQ ID NO:3; the Tyro3 engulfment signaling domain is truncated 1, 2, 3, 4, 5, or more amino acids at its N-terminus corresponding to the amino acid sequence of SEQ ID NO:5; the Ax1 engulfment signaling domain is truncated 1, 2, 3, 4, 5, or more amino acids at its N-terminus corresponding to the amino acid sequence of SEQ ID NO:6; the ELMO engulfment signaling domain is truncated 1, 2, 3, 4, 5, or more amino acids at its N-terminus corresponding to the amino acid sequence of SEQ ID NO:7; the Traf6 engulfment signaling domain is truncated 1, 2, 3, 4, 5, or more amino acids at its N-terminus corresponding to the amino acid sequence of SEQ ID NO:8; the Syk engulfment signaling domain is truncated 1, 2, 3, 4, 5, or more amino acids at its N-terminus corresponding to the amino acid sequence of SEQ ID NO:9; the MyD88 engulfment signaling domain is truncated 1, 2, 3, 4, 5, or more amino acids at its N-terminus corresponding to the amino acid sequence of SEQ ID NO:10; the PI3K engulfment signaling domain is truncated 1, 2, 3, 4, 5, or more amino acids at its N-terminus corresponding to the amino acid sequence of SEQ ID NO:11; the FcεRIγ engulfment signaling domain is truncated 1, 2, 3, 4, 5, or more amino acids at its N-terminus corresponding to the amino acid sequence of SEQ ID NO:12; the FcγR1 engulfment signaling domain is truncated 1, 2, 3, 4, 5, or more amino acids at its N-terminus corresponding to the amino acid sequence of SEQ ID NO:13; the FcγR2A engulfment signaling domain is truncated 1, 2, 3, 4, 5, or more amino acids at its N-terminus corresponding to the amino acid sequence of SEQ ID NO:14; the FcγR2C engulfment signaling domain is truncated 1, 2, 3, 4, 5, or more amino acids at its N-terminus corresponding to the amino acid sequence of SEQ ID NO:15; the FcγR3A engulfment signaling domain is truncated 1, 2, 3, 4, 5, or more amino acids at its N-terminus corresponding to the amino acid sequence of SEQ ID NO:16; the BAFF-R engulfment signaling domain is truncated 1, 2, 3, 4, 5, or more amino acids at its N-terminus corresponding to the amino acid sequence of SEQ ID NO:17; the DAP-12 engulfment signaling domain is truncated 1, 2, 3, 4, 5, or more amino acids at its N-terminus corresponding to the amino acid sequence of SEQ ID NO:18; the NFAM1 engulfment signaling domain is truncated 1, 2, 3, 4, 5, or more amino acids at its N-terminus corresponding to the amino acid sequence of SEQ ID NO:19; the CD79b engulfment signaling domain is truncated 1, 2, 3, 4, 5, or more amino acids at its N-terminus corresponding to the amino acid sequence of SEQ ID NO:21; the TLR1 engulfment signaling domain is truncated 1, 2, 3, 4, 5, or more amino acids at its N-terminus corresponding to the amino acid sequence of SEQ ID NO:22; the TLR2 engulfment signaling domain is truncated 1, 2, 3, 4, 5, or more amino acids at its N-terminus corresponding to the amino acid sequence of SEQ ID NO:23; the TLR3 engulfment signaling domain is truncated 1, 2, 3, 4, 5, or more amino acids at its N-terminus corresponding to the amino acid sequence of SEQ ID NO:24; the TLR4 engulfment signaling domain is truncated 1, 2, 3, 4, 5, or more amino acids at its N-terminus corresponding to the amino acid sequence of SEQ ID NO:25; the TLR5 engulfment signaling domain is truncated 1, 2, 3, 4, 5, or more amino acids at its N-terminus corresponding to the amino acid sequence of SEQ ID NO:26; the TLR6 engulfment signaling domain is truncated 1, 2, 3, 4, 5, or more amino acids at its N-terminus corresponding to the amino acid sequence of SEQ ID NO:27; the TLR7 engulfment signaling domain is truncated 1, 2, 3, 4, 5, or more amino acids at its N-terminus corresponding to the amino acid sequence of SEQ ID NO:28; the TLR8 engulfment signaling domain is truncated 1, 2, 3, 4, 5, or more amino acids at its N-terminus corresponding to the amino acid sequence of SEQ ID NO:29; the TLR9 engulfment signaling domain is truncated 1, 2, 3, 4, 5, or more amino acids at its N-terminus corresponding to the amino acid sequence of SEQ ID NO:30; the Traf2 engulfment signaling domain is truncated 1, 2, 3, 4, 5, or more amino acids at its N-terminus corresponding to the amino acid sequence of SEQ ID NO:31; or the Traf3 engulfment signaling domain is truncated 1, 2, 3, 4, 5, or more amino acids at its N-terminus corresponding to the amino acid sequence of SEQ ID NO:32.

In certain embodiments, the MRC1 engulfment signaling domain is truncated 1, 2, 3, 4, 5, or more amino acids at its C-terminus corresponding to the amino acid sequence of SEQ ID NO:2; the MERTK engulfment signaling domain is truncated 1, 2, 3, 4, 5, or more amino acids at its C-terminus corresponding to the amino acid sequence of SEQ ID NO:3; the Tyro3 engulfment signaling domain is truncated 1, 2, 3, 4, 5, or more amino acids at its C-terminus corresponding to the amino acid sequence of SEQ ID NO:5; the Ax1 engulfment signaling domain is truncated 1, 2, 3, 4, 5, or more amino acids at its C-terminus corresponding to the amino acid sequence of SEQ ID NO:6; the ELMO engulfment signaling domain is truncated 1, 2, 3, 4, 5, or more amino acids at its C-terminus corresponding to the amino acid sequence of SEQ ID NO:7; the Traf6 engulfment signaling domain is truncated 1, 2, 3, 4, 5, or more amino acids at its C-terminus corresponding to the amino acid sequence of SEQ ID NO:8; the Syk engulfment signaling domain is truncated 1, 2, 3, 4, 5, or more amino acids at its C-terminus corresponding to the amino acid sequence of SEQ ID NO:9; the MyD88 engulfment signaling domain is truncated 1, 2, 3, 4, 5, or more amino acids at its C-terminus corresponding to the amino acid sequence of SEQ ID NO:10; the PI3K engulfment signaling domain is truncated 1, 2, 3, 4, 5, or more amino acids at its C-terminus corresponding to the amino acid sequence of SEQ ID NO:11; the FcεRIγ engulfment signaling domain is truncated 1, 2, 3, 4, 5, or more amino acids at its C-terminus corresponding to the amino acid sequence of SEQ ID NO:12; the FcγR1 engulfment signaling domain is truncated 1, 2, 3, 4, 5, or more amino acids at its C-terminus corresponding to the amino acid sequence of SEQ ID NO:13; the FcγR2A engulfment signaling domain is truncated 1, 2, 3, 4, 5, or more amino acids at its C-terminus corresponding to the amino acid sequence of SEQ ID NO:14; the FcγR2C engulfment signaling domain is truncated 1, 2, 3, 4, 5, or more amino acids at its C-terminus corresponding to the amino acid sequence of SEQ ID NO:15; the FcγR3A engulfment signaling domain is truncated 1, 2, 3, 4, 5, or more amino acids at its C-terminus corresponding to the amino acid sequence of SEQ ID NO:16; the BAFF-R engulfment signaling domain is truncated 1, 2, 3, 4, 5, or more amino acids at its C-terminus corresponding to the amino acid sequence of SEQ ID NO:17; the DAP-12 engulfment signaling domain is truncated 1, 2, 3, 4, 5, or more amino acids at its C-terminus corresponding to the amino acid sequence of SEQ ID NO:18; the NFAM1 engulfment signaling domain is truncated 1, 2, 3, 4, 5, or more amino acids at its C-terminus corresponding to the amino acid sequence of SEQ ID NO:19; the CD79b engulfment signaling domain is truncated 1, 2, 3, 4, 5, or more amino acids at its C-terminus corresponding to the amino acid sequence of SEQ ID NO:21; the TLR1 engulfment signaling domain is truncated 1, 2, 3, 4, 5, or more amino acids at its C-terminus corresponding to the amino acid sequence of SEQ ID NO:22; the TLR2 engulfment signaling domain is truncated 1, 2, 3, 4, 5, or more amino acids at its C-terminus corresponding to the amino acid sequence of SEQ ID NO:23; the TLR3 engulfment signaling domain is truncated 1, 2, 3, 4, 5, or more amino acids at its C-terminus corresponding to the amino acid sequence of SEQ ID NO:24; the TLR4 engulfment signaling domain is truncated 1, 2, 3, 4, 5, or more amino acids at its C-terminus corresponding to the amino acid sequence of SEQ ID NO:25; the TLR5 engulfment signaling domain is truncated 1, 2, 3, 4, 5, or more amino acids at its C-terminus corresponding to the amino acid sequence of SEQ ID NO:26; the TLR6 engulfment signaling domain is truncated 1, 2, 3, 4, 5, or more amino acids at its C-terminus corresponding to the amino acid sequence of SEQ ID NO:27; the TLR7 engulfment signaling domain is truncated 1, 2, 3, 4, 5, or more amino acids at its C-terminus corresponding to the amino acid sequence of SEQ ID NO:28; the TLR8 engulfment signaling domain is truncated 1, 2, 3, 4, 5, or more amino acids at its C-terminus corresponding to the amino acid sequence of SEQ ID NO:29; the TLR9 engulfment signaling domain is truncated 1, 2, 3, 4, 5, or more amino acids at its C-terminus corresponding to the amino acid sequence of SEQ ID NO:30; the Traf2 engulfment signaling domain is truncated 1, 2, 3, 4, 5, or more amino acids at its C-terminus corresponding to the amino acid sequence of SEQ ID NO:31; or the Traf3 engulfment signaling domain is truncated 1, 2, 3, 4, 5, or more amino acids at its C-terminus corresponding to the amino acid sequence of SEQ ID NO:32.

In certain embodiments, a truncated MyD88 engulfment signaling domain comprises a death domain but lacks a Toll/interleukin-1 receptor (TIR) homology domain. An example of such a truncated MyD88 engulfment signaling domain comprises an amino acid sequence of SEQ ID NO:33. In certain embodiments, a truncated MyD88 engulfment signaling domain comprises a TIR domain. An example of a truncated MyD88 engulfment signaling domain comprising a TIR domain comprises an amino acid sequence of SEQ ID NO:106. An exemplary truncated Traf6 signaling domain comprises an amino acid sequence of SEQ ID NO:34. An exemplary truncated NFAM1 signaling domain comprises an amino acid sequence of SEQ ID NO:35. An exemplary truncated CD79b signaling domain comprises an amino acid sequence of SEQ ID NO:20.

In certain embodiments, a CER comprises a first engulfment signaling domain and a second engulfment signaling domain. In some embodiments, a CER comprises a first engulfment signaling domain and a second engulfment signaling domain that are from the same molecule. In other embodiments, the first engulfment signaling domain and the second engulfment signaling domain are from different molecules.

Engulfment signaling domains may be derived from a mammalian species, including humans, primates, cows, horses, goats, sheep, dogs, cats, mice, rats, rabbits, guinea pigs, pigs, and transgenic species thereof.

### Transmembrane Domains

CERs of the present disclosure comprise a transmembrane domain that connects and is positioned between the extracellular domain and the engulfment signaling domain. The transmembrane domain is a hydrophobic alpha helix that transverses the host cell membrane and anchors the CER in the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be selected from the same molecule as the extracellular domain or the engulfment signaling domain (e.g., a CER comprising a TLR4 engulfment signaling domain and a TLR4 transmembrane domain or a CER comprising a Tim4 binding domain and a Tim4 transmembrane domain). In certain embodiments, the transmembrane domain and the extracellular domain are each selected from different molecules. In other embodiments, the transmembrane domain and the engulfment signaling domain are each selected from different molecules. In yet other embodiments, the transmembrane domain, the extracellular domain, and the engulfment signaling domain are each selected from different molecules.

In certain embodiments, the transmembrane domain comprises aTim1, Tim4, Tim3, FcR (e.g., FcγR1, FcγR2A, FcγR2B2, FcγR2C, FcγR3A, FcεR1, or FcαR1), CD8a, CD28, MERTK, Axl, Tyro3, CD4, DAP12, MRC1, TLR1, TLR2, TLR3, TLR4, TLR5, TLR6, TLR7, TLR8, or TLR9 transmembrane domain.

In certain embodiments, the transmembrane domain comprises a sequence that has at least about 75%, 80%, 85%, 90%, 91%, 92%, 93%, 94%, 95%, 96%, 97%, 98%, 99%, 99.5%, or 100% identity to a Timl transmembrane domain comprising an amino acid sequence of SEQ ID NO:36, Tim4 transmembrane domain comprising an amino acid sequence of SEQ ID NO:37 or 38, Tim3 transmembrane domain comprising an amino acid sequence of SEQ ID NO:39, FcγR1 transmembrane domain comprising an amino acid sequence of SEQ ID NO:40, FcγR2A transmembrane domain comprising an amino acid sequence of SEQ ID NO:41, FcγR2B2 transmembrane domain comprising an amino acid sequence of SEQ ID NO:42, FcγR2C transmembrane domain comprising an amino acid sequence of SEQ ID NO:43, FcγR3A transmembrane domain comprising an amino acid sequence of SEQ ID NO:44, FcεR1 transmembrane domain comprising an amino acid sequence of SEQ ID NO:45, FcαR1 transmembrane domain comprising an amino acid sequence of SEQ ID NO:46, CD8a transmembrane domain comprising an amino acid sequence of SEQ ID NO:47, CD28 transmembrane domain comprising an amino acid sequence of SEQ ID NO:107, MERTK transmembrane domain comprising an amino acid sequence of SEQ ID NO:48, Ax1 transmembrane domain comprising an amino acid sequence of SEQ ID NO:49, Tyro3 transmembrane domain comprising an amino acid sequence of SEQ ID NO:50, CD4 transmembrane domain comprising an amino acid sequence of SEQ ID NO:51, DAP12 transmembrane domain comprising an amino acid sequence of SEQ ID NO:52, MRC1 transmembrane domain comprising an amino acid sequence of SEQ ID NO:53, TLR1 transmembrane domain comprising an amino acid sequence of SEQ ID NO:54, TLR2 transmembrane domain comprising an amino acid sequence of SEQ ID NO:55, TLR3 transmembrane domain comprising an amino acid sequence of SEQ ID NO:56, TLR4 transmembrane domain comprising an amino acid sequence of SEQ ID NO:57, TLR5 transmembrane domain comprising an amino acid sequence of SEQ ID NO:58, TLR6 transmembrane domain comprising an amino acid sequence of SEQ ID NO:59, TLR7 transmembrane domain comprising an amino acid sequence of SEQ ID NO:60, TLR8 transmembrane domain comprising an amino acid sequence of SEQ ID NO:61, or TLR9 transmembrane domain comprising an amino acid sequence of SEQ ID NO:62.

In certain embodiments, the transmembrane domain is a Tim1 transmembrane domain comprising or consisting of an amino acid sequence of SEQ ID NO:36, Tim4 transmembrane domain comprising or consisting of an amino acid sequence of SEQ ID NO:37 or 38, Tim3 transmembrane domain comprising or consisting of an amino acid sequence of SEQ ID NO:39, FcγR1 transmembrane domain comprising or consisting of an amino acid sequence of SEQ ID NO:40, FcγR2A transmembrane domain comprising or consisting of an amino acid sequence of SEQ ID NO:41, FcγR2B2 transmembrane domain comprising or consisting of an amino acid sequence of SEQ ID NO:42, FcγR2C transmembrane domain comprising or consisting of an amino acid sequence of SEQ ID NO:43, FcγR3A transmembrane domain comprising or consisting of an amino acid sequence of SEQ ID NO:44, FcεR1 transmembrane domain comprising or consisting of an amino acid sequence of SEQ ID NO:45, FcαR1 transmembrane domain comprising or consisting of an amino acid sequence of SEQ ID NO:46, CD8a transmembrane domain comprising or consisting of an amino acid sequence of SEQ ID NO:47, CD28 transmembrane domain comprising or consisting of an amino acid sequence of SEQ ID NO:107, MERTK transmembrane domain comprising or consisting of an amino acid sequence of SEQ ID NO:48, Ax1 transmembrane domain comprising or consisting of an amino acid sequence of SEQ ID NO:49, Tyro3 transmembrane domain comprising or consisting of an amino acid sequence of SEQ ID NO:50, CD4 transmembrane domain comprising or consisting of an amino acid sequence of SEQ ID NO:51, DAP12 transmembrane domain comprising or consisting of an amino acid sequence of SEQ ID NO:52, MRC1 transmembrane domain comprising or consisting of an amino acid sequence of SEQ ID NO:53, TLR1 transmembrane domain comprising or consisting of an amino acid sequence of SEQ ID NO:54, TLR2 transmembrane domain comprising or consisting of an amino acid sequence of SEQ ID NO:55, TLR3 transmembrane domain comprising or consisting of an amino acid sequence of SEQ ID NO:56, TLR4 transmembrane domain comprising or consisting of an amino acid sequence of SEQ ID NO:57, TLR5 transmembrane domain comprising or consisting of an amino acid sequence of SEQ ID NO:58, TLR6 transmembrane domain comprising or consisting of an amino acid sequence of SEQ ID NO:59, TLR7 transmembrane domain comprising or consisting of an amino acid sequence of SEQ ID NO:60, TLR8 transmembrane domain comprising or consisting of an amino acid sequence of SEQ ID NO:61, or TLR9 transmembrane domain comprising or consisting of an amino acid sequence of SEQ ID NO:62.

Transrnembrane domains may derived from any mammalian species, including humans, primates, cows, horses, goats, sheep, dogs, cats, mice, rats, rabbits, guinea pigs, pigs, and transgenic species thereof.

In certain embodiments, a chimeric engulfment receptor comprises polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, a chimeric engulfment receptor is murine, chimeric, human, or humanized.

It is understood that direct fusion of one domain to another domain of a CER described herein does not preclude the presence of intervening junction amino acids. Junction amino acids may be natural or non-natural (e.g., resulting from the construct design of a chimeric protein).

Embodiments of CERs for use in the tandem expression cassettes of the present disclosure are provided in Table 1 and also described in PCT Application Nos. PCT/2017/053553 and PCT/US2018/52297, each of which is incorporated by reference in its entirety.

TABLE 1
Exemplary Chimeric Engulfment Receptors

| | | | First | Second | Ex- |
|---|---|---|---|---|---|
| | | Trans- | Engulf- | Engulf- | emplary |
| | | mem- | ment | ment | Amino |
| CER | Binding | brane | Signaling | Signaling | Acid |
| Name | Domain | Domain | Domain | Domain | Sequences |
| CER1 | Tim4 | Tim4 | MERTK | | SEQ ID |
| | | | | | NO: 109 |
| CER5 | Tim4 | Tim4 | TLR4 | | SEQ ID |
| | | | | | NO: 97 |
| CER6 | Tim4 | TLR4 | TLR4 | | SEQ ID |
| | | | | | NO: 110 |
| CER7 | Tim4 + | TLR4 | TLR4 | | SEQ ID |
| | TLR | | | | NO: 111 |
| | juxta- | | | | |
| | membrane | | | | |
| | domain | | | | |
| CER8 | Tim4 | Tim4 | Tyro3 | | SEQ ID |
| | | | | | NO: 112 |
| CER9 | Tim4 | Tim4 | DAP12 | | SEQ ID |
| | | | | | NO: 113 |
| CER10 | Tim4 | DAP12 | DAP12 | | SEQ ID |
| | | | | | NO: 114 |
| CER11 | Tim4 | Tim4 | Axl | | SEQ ID |
| | | | | | NO: 115 |
| CER12 | Tim4 | Tim4 | FcRg | | SEQ ID |
| | | | | | NO: 116 |
| CER13 | Tim4 | FcϵR1 | FcRg | | SEQ ID |
| | | | | | NO: 117 |
| CER15 | Tim4 | Tim4 | MyD88 | | SEQ ID |
| | | | | | NO: 118 |
| CER16 | Tim4 | Tim4 | MyD88_ | | SEQ ID |
| | | | TIR | | NO: 119 |
| CER17 | Tim4 | Tim4 | TLR_3 | | SEQ ID |
| | | | | | NO: 120 |
| CER18 | Tim4 | TLR_3 | TLR_3 | | SEQ ID |
| | | | | | NO: 121 |
| CER19 | Tim4 | Tim4 | TLR_5 | | SEQ ID |
| | | | | | NO: 98 |
| CER20 | Tim4 | TLR_5 | TLR_5 | | SEQ ID |
| | | | | | NO: 122 |
| CER21 | Tim4 | Tim4 | TLR_8 | | SEQ ID |
| | | | | | NO: 99 |
| CER22 | Tim4 | TLR_8 | TLR_8 | | SEQ ID |
| | | | | | NO: 123 |
| CER23 | Tim4 | Tim4 | TLR_9 | | SEQ ID |
| | | | | | NO: 124 |
| CER24 | Tim4 | TLR_9 | TLR_9 | | SEQ ID |
| | | | | | NO: 125 |
| CER25 | Tim4 | Tim4 | NFAM | | SEQ ID |
| | | | | | NO: 100 |
| CER26 | Tim4 | Tim4 | TLR_1 | | SEQ ID |
| | | | | | NO: 126 |
| CER27 | Tim4 | Tim4 | TLR_2 | | SEQ ID |
| | | | | | NO: 101 |
| CER28 | Tim4 | Tim4 | TLR_7 | | SEQ ID |
| | | | | | NO: 127 |
| CER29 | Tim4 | Tim4 | TRAF6 | | SEQ ID |
| | | | | | NO: 102 |
| CER30 | Tim4 | Tim4 | TRAF2 | | SEQ ID |
| | | | | | NO: 128 |
| CER31 | Tim4 | Tim4 | TRAF3 | | SEQ ID |
| | | | | | NO: 103 |
| CER85 | Tim4 | Tim4 | MyD88 | Bafr-R | SEQ ID |
| | | | | | NO: 129 |
| CER86 | Tim4 | Tim4 | MyD88 | DAP12 | SEQ ID |
| | | | | | NO: 130 |
| CER87 | Tim4 | Tim4 | Baff-R | MyD88 | SEQ ID |
| | | | | | NO: 131 |
| CER88 | Tim4 | Tim4 | DAP12 | MyD88 | SEQ ID |
| | | | | | NO: 132 |
| CER89 | Tim4 | Tim4 | MyD88 | CD79b | SEQ ID |
| | | | | (185-229) | NO: 133 |
| CER90 | Tim4 | Tim4 | MyD88 | NFAM1 | SEQ ID |
| | | | | | NO: 134 |
| CER91 | Tim4 | Tim4 | MyD88 | P2A-Rab | SEQ ID |
| | | | | | NO: 135 |
| CER92 | Tim4 | Tim4 | MERTK | MyD88 | SEQ ID |
| | | | | | NO: 136 |
| CER93 | Tim4 | Tim4 | MERTK | Baff-R | SEQ ID |
| | | | | | NO: 137 |
| CER94 | Tim4 | Tim4 | MERTK | DAP12 | SEQ ID |
| | | | | | NO: 138 |
| CER95 | Tim4 | Tim4 | MERTK | CD79b | SEQ ID |
| | | | | (185-229) | NO: 139 |
| CER96 | Tim4 | Tim4 | MERTK | NFAM1 | SEQ ID |
| | | | | | NO: 140 |
| CER97 | Tim4 | Tim4 | AXL | Dap12 | SEQ ID |
| | | | | | NO: 141 |
| CER98 | Tim4 | Tim4 | AXL | CD79b | SEQ ID |
| | | | | | NO: 142 |
| CER99 | Tim4 | Tim4 | AXL | NFAM1 | SEQ ID |
| | | | | | NO: 143 |
| CER102 | Tim4 | Tim4 | TLR8 | NFAM1 | SEQ ID |
| | | | | | NO: 144 |
| CER103A | Tim4 | Tim4 | TLR8 | CD79b | SEQ ID |
| | | | | (185-229) | NO: 145 |
| CER103B | Tim4 | Tim4 | TLR8 | CD79b | SEQ ID |
| | | | | (185-213) | NO: 146 |
| CER104 | Tim4 | Tim4 | TLR8 | DAP12 | SEQ ID |
| | | | | | NO: 147 |
| CER105 | Tim4 | Tim4 | TLR8 | Baff-R | SEQ ID |
| | | | | | NO: 148 |
| CER106 | Tim4 | Tim4 | NFAM1 | TLR8 | SEQ ID |
| | | | | | NO: 149 |
| CER107 | Tim4 | Tim4 | CD79b | TLR8 | SEQ ID |
| | | | (185-213) | | NO: 150 |
| CER108 | Tim4 | Tim4 | DAP12 | TLR8 | SEQ ID |
| | | | | | NO: 151 |
| CER109 | Tim4 | Tim4 | Baff-R | TLR8 | SEQ ID |
| | | | | | NO: 152 |
| CER110 | Tim4 | Tim4 | TRAF6 | DAP12 | SEQ ID |
| | | | | | NO: 153 |
| CER111A | Tim4 | Tim4 | TRAF6 | CD79b | SEQ ID |
| | | | | (185-229) | NO: 154 |
| CER111B | Tim4 | Tim4 | TRAF6 | CD79b | SEQ ID |
| | | | | (185-213) | NO: 155 |
| CER112 | Tim4 | Tim4 | TRAF6 | NFAM1 | SEQ ID |
| | | | | | NO: 156 |
| CER113 | Tim4 | Tim4 | TRAF6 | Baff-R | SEQ ID |
| | | | | | NO: 157 |
| CER114 | Tim4 | Tim4 | TRAF6 | MERTK | SEQ ID |
| | | | | | NO: 158 |
| CER115 | Tim4 | Tim4 | MERTK | TRAF6 | SEQ ID |
| | | | | | NO: 159 |
| CER116 | Tim4 | Tim4 | TRAF6 | TLR8 | SEQ ID |
| | | | | | NO: 160 |
| CER117 | Tim4 | Tim4 | TLR8 | TRAF6 | SEQ ID |
| | | | | | NO: 161 |
| CER118 | Tim4 | Tim4 | TLR1 | NFAM1 | SEQ ID |
| | | | | | NO: 162 |
| CER119A | Tim4 | Tim4 | TLR1 | CD79b | SEQ ID |
| | | | | (185-229) | NO: 183 |
| CER119B | Tim4 | Tim4 | TLR1 | CD79b | SEQ ID |
| | | | | (185-213) | NO: 163 |
| CER120 | Tim4 | Tim4 | TLR1 | DAP12 | SEQ ID |
| | | | | | NO: 164 |
| CER121 | Tim4 | Tim4 | TLR1 | TRAF6 | SEQ ID |
| | | | | | NO: 165 |
| CER122 | Tim4 | Tim4 | TLR2 | DAP12 | SEQ ID |
| | | | | | NO: 171 |
| CER123 | Tim4 | Tim4 | TLR2 | TRAF6 | SEQ ID |
| | | | | | NO: 172 |
| CER124 | Tim4 | Tim4 | TLR2 | NFAM1 | SEQ ID |
| | | | | | NO: 173 |
| CER125A | Tim4 | Tim4 | TLR2 | CD79b | SEQ ID |
| | | | | (185-229) | NO: 174 |
| CER125B | Tim4 | Tim4 | TLR2 | CD79b | SEQ ID |
| | | | | (185-213 | NO: 175 |
| CER126 | Tim4 | Tim4 | TLR2 | TRAF2 | SEQ ID |
| | | | | | NO: 184 |
| CER127 | Tim4 | Tim4 | TRAF2 | TLR2 | SEQ ID |
| | | | | | NO: 185 |
| CER128 | Tim4 | Tim4 | TRAF2 | TLR8 | SEQ ID |
| | | | | | NO: 186 |
| CER129 | Tim4 | Tim4 | TLR8 | TRAF2 | SEQ ID |
| | | | | | NO: 187 |
| hCER21 | Tim4 | Tim4 | TLR8 | | SEQ ID |
| | | | | | NO: 188 |
| hCER29 | Tim4 | Tim4 | TRAF6 | | SEQ ID |
| | | | | | NO: 189 |
| hCER104 | Tim4 | Tim4 | TLR8 | Dap12 | SEQ ID |
| | | | | | NO: 190 |
| hCER116 | Tim4 | Tim4 | TRAF6 | TLR8 | SEQ ID |
| | | | | | NO: 191 |
| hCER117 | Tim4 | Tim4 | TLR8 | TRAF6 | SEQ ID |
| | | | | | NO: 192 |
| hCER122 | Tim4 | Tim4 | TLR2 | Dap12 | SEQ ID |
| | | | | | NO: 193 |
| hCER123 | Tim4 | Tim4 | TLR2 | TRAF6 | SEQ ID |
| | | | | | NO: 194 |
| hCER126 | Tim4 | Tim4 | TLR2 | TRAF2 | SEQ ID |
| | | | | | NO: 195 |

### II. Chimeric Antigen Receptors

In certain embodiments, tandem expression cassettes of the present disclosure comprise a transgene encoding a chimeric antigen receptor (CAR). Chimeric antigen receptors are recombinant receptors that generally comprise: an extracellular domain comprising a binding domain that binds to a target antigen; an intracellular signaling domain, and a transmembrane domain positioned between and connecting the extracellular domain and the intracellular signaling domain.

Binding domains suitable for use in CARs of the present disclosure include any antigen-binding polypeptide. A binding domain may comprise an antibody or antigen binding fragment thereof, including for example, a full length heavy chain, Fab fragment, Fab′, F(ab′)₂, sFv, VH domain, VL domain, dAb, VHH, CDR, and scFv. In certain embodiments, a CAR binding domain is murine, chimeric, human, or humanized.

In certain embodiments, the extracellular domain of CARs provided in the present disclosure optionally comprises an extracellular, non-signaling spacer or linker domain. Where included, such a spacer or linker domain may position the binding domain away from the host cell surface to further enable proper cell to cell contact, binding, and activation. An extracellular spacer domain is generally located between the extracellular binding domain and the transmembrane domain of the CAR. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG₄ hinge region having an amino acid sequence of ESKYGPPCPPCP (SEQ ID NO:63).

Other examples of hinge regions that may be used in the CARs described herein include the hinge region from the extracellular regions of type 1 membrane proteins, such as CD8a, CD4, CD28 and CD7, which may be wild-type or variants thereof. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

CARs of the present disclosure comprise a transmembrane domain that connects and is positioned between the extracellular domain and the intracellular signaling domain. The transmembrane domain is a hydrophobic alpha helix that transverses the host cell membrane and anchors the CAR in the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be selected from the same molecule as the extracellular domain or the intracellular signaling domain (e.g., a CAR comprises a CD28 costimulatory signaling domain and a CD28 transmembrane domain). In certain embodiments, the transmembrane domain and the extracellular domain are each selected from different molecules. In other embodiments, the transmembrane domain and the intracellular signaling domain are each selected from different molecules. In yet other embodiments, the transmembrane domain, the extracellular domain, and the intracellular signaling domain are each selected from different molecules.

Exemplary transmembrane domains for use in CARs of the present disclosure include CD28, CD2, CD3ε, CD3ζ, CD3, CD25, CD27, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, and Zap70. An exemplary CD28 transmembrane domain comprises an amino acid sequence of SEQ ID NO:107.

The intracellular signaling domain of a CAR is an intracellular effector domain and is capable of transmitting functional signals to a cell in response to binding of the extracellular domain of the CAR to a target molecule. The intracellular signaling domain may be any portion of an intracellular signaling molecule that retains sufficient signaling activity. In some embodiments, a full length or full length intracellular component of an intracellular signaling molecule is used. In some embodiments, a truncated portion of an intracellular signaling molecule or intracellular component of an intracellular signaling molecule is used, provided that the truncated portion retains sufficient signal transduction activity. In further embodiments, an intracellular signaling domain is a variant of an entire or truncated portion of an intracellular signaling molecule, provided that the variant retains sufficient signal transduction activity (i.e., is a functional variant).

In certain embodiments, the intracellular signaling domain of a CAR comprises an immunoreceptor tyrosine-based activation motif (ITAM) containing signaling domain. An ITAM containing signaling domain generally contains at least one (one, two, three, four, or more) ITAMs, which refer to a conserved motif of YXXL/I-X₆₋₈-YXXL/I. An ITAM containing signaling domain may initiate T cell activation signaling following antigen binding or ligand engagement. ITAM-signaling domains include, for example, intracellular signaling domains of CD3γ, CD3δ, CD3ε, CD3ζ, CD5, CD22, CD79a, CD278 (ICOS), DAP10, DAP12, and CD66d. Exemplary CD3ζ signaling domains that may be used in CARs of the present disclosure comprise an amino acid sequence of SEQ ID NO:166 or 167.

CAR intracellular signaling domains optionally comprise a costimulatory signaling domain, which, when activated in conjunction with a primary or classic (e.g., ITAM-driven) activation signal, promotes or enhances T cell response, such as T cell activation, cytokine production, proliferation, differentiation, survival, effector function, or combinations thereof. Costimulatory signaling domains for use in CARs include, for example, CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, SLP76, TRIM, ZAP70, or any combination thereof In a particular embodiment, the costimulatory signaling domain comprises a OX40, CD2, CD27, CD28, ICAM-1, LFA-1 (CD11a/CD18), ICOS (CD278), or 4-1BB (CD137) signaling domain. Exemplary CD28 costimulatory signaling domains that may be used in CARs of the present disclosure comprise an amino acid sequence of SEQ ID NO:169 or 170. An exemplary 4-1BB costimulatory signaling domain comprises an amino acid sequence of SEQ ID NO:168. In certain embodiments, a CAR comprises one, two, or more costimulatory signaling domains.

In certain embodiments, a chimeric antigen receptor comprises polynucleotide sequences derived from any mammalian species, including humans, primates, cows, horses, goats, sheep, dogs, cats, mice, rats, rabbits, guinea pigs, pigs, transgenic species thereof, or any combination thereof In certain embodiments, chimeric antigen receptor is murine, chimeric, human, or humanized

In certain embodiments, a CAR is a first generation CAR, a second generation CAR, or a third generation CAR. A first generation CAR generally has an intracellular signaling domain comprising an intracellular signaling domain of CD3ζ, FcγRI, or other ITAM-containing activating domain to provide a T cell activation signal. Second generation CARs further comprise a costimulatory signaling domain (e.g., a costimulatory signaling domain from an endogenous T cell costimulatory receptor, such as CD28, 4-1BB, or ICOS). Third generation CARs comprise an ITAM-containing activating domain, a first costimulatory signaling domain and a second costimulatory signaling domain.

In certain embodiments, a CAR is a T cell receptor-based chimeric antigen receptor (TCR-CAR). A TCR-CAR is a heterodimeric fusion protein generally comprising a soluble TCR (a polypeptide chain comprising a Vα domain and Cα domain and a polypeptide chain comprising a Vβ domain and a Cβ domain), wherein the VβCβ polypeptide chain is linked to a transmembrane domain and an intracellular signaling component (e.g., an ITAM-containing activating domain and optionally a costimulatory signaling domain) (see, e.g., Walseng et al., 2017 Scientific Reports 7:10713).

CARs of the present disclosure may target a variety of antigens, including a viral antigen, bacterial antigen, fungal antigen, parasitic antigen, tumor antigen, autoimmune disease antigen. Exemplary tumor antigens that a CAR may target include CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor a, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2.

### III. T Cell Receptor Binding Proteins

In certain embodiments, tandem expression cassettes of the present disclosure comprise a transgene encoding a recombinant TCR binding protein. Recombinant TCR binding proteins include “traditional” TCRs composed of a heterodimer of α chain polypeptide and β chain polypeptide or a heterodimer of a γ chain polypeptide and a δ chain polypeptide, binding fragments and fusion proteins thereof, including for example, single chain TCRs, single domain TCRs, soluble TCR fusion TCR proteins, and TCR fusion constructs (TRuC™). In certain embodiments, a tandem expression cassette comprises a polynucleotide encoding a recombinant TCR beta chain comprising a TCR beta variable region and a TCR beta constant region, and a polynucleotide encoding a recombinant TCR alpha chain comprising a TCR alpha variable region and a TCR alpha constant region. In certain embodiments, a recombinant TCR is an enhanced affinity TCR.

In certain embodiments, a recombinant TCR binding protein is a single chain TCR (scTCR) comprising a Va joined to a Vβ by a flexible linker. In some embodiments, a scTCR comprises a Vα-linker-Vβ polypeptide. In other embodiments, a scTCR comprises a Vβ-linker-Vα polypeptide.

In certain embodiments, a recombinant TCR binding protein is a single domain TCR (e.g., Vβ).

In certain embodiments, a recombinant TCR binding protein is a single chain TCR (scTCR) fusion protein. A scTCR fusion protein comprises a binding domain comprising a scTCR (a TCR Va domain linked to a TCR Vβ domain), an optional extracellular spacer, a transmembrane domain, and an intracellular signaling domain comprising a CD3ζITAM-containing activating domain and optionally a costimulatory signaling domain (see, Aggen et al., 2012, Gene Ther. 19:365-374; Stone et al., Cancer Immunol. Immunother. 2014, 63:1163-76).

In certain embodiments, a recombinant TCR binding protein is a TCR fusion construct (TRuC™ construct) (see, U.S. Patent Publication No. 2017/0166622). TRuC™ constructs comprise an antigen-specific binding domain (e.g., scFv) fused at least one component of a TCR complex (CD3γ, CD3ε, or CD3δ) to form a TCR complex component fusion protein. A human TCR complex contains the CD3ε polypeptide, the CD3γ polypeptide, the CD3δ polypeptide, the CD3ζ polypeptide, the TCR α chain polypeptide, and the TCR β chain polypeptide. The TCR complex component fusion protein is capable of associating with the other components of the TCR complex to form a functional, complete TCR fusion complex. Unlike TCRs, TRuC™ constructs are capable of binding a target antigen in a MEW independent manner.

In certain embodiments, a TCR binding protein comprises polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, the TCR binding protein is murine, chimeric, human, or humanized.

TCR binding proteins of the present disclosure may bind to a variety of antigens, including tumor antigens, viral antigens, bacterial antigens, fungal antigens, parasitic antigens, and autoimmune disease antigens. Exemplary tumor antigens that a recombinant TCR binding protein may target include WT-1, mesothelin, MART-1, NY-ESO-1, MAGE-A3, HPV E7, survivin, a Fetoprotein, and a tumor-specific neoantigen. Exemplary HPV16 E7 specific TCRs that may be used in tandem expression cassettes of the present disclosure are provided in PCT Published Application No. WO2015/184228 (incorporated by reference in its entirety). In certain embodiments, a HPV16 E7 specific TCR comprises an amino acid sequence of SEQ ID NO:90. The amino acid sequence of SEQ ID NO:90 contains a P2A self-cleaving peptide between the TCRβ chain sequence and the TCRα chain sequence, which would be cleaved in the host cell to form two polypeptide chains. Thus, in certain embodiments, the TCR represented by SEQ ID NO:90 comprises separate TCRβ and TCRα polypeptide chains that are capable dimerizing to form a αβTCR. In certain embodiments, a HPV16 E7 specific TCR comprises a Vβ comprising an amino acid sequence of SEQ ID NO:92. In certain embodiments, a HPV16 E7 specific TCR comprises a Va comprising an amino acid sequence of SEQ ID NO:94. In further embodiments, a HPV16 specific E7 TCR comprises a Vβ comprising an amino acid sequence of SEQ ID NO:92 and a Vα comprising an amino acid sequence of SEQ ID NO:94.

In certain embodiments, a TCR Cα domain, a Cβ domain, or both comprise a cysteine substitution to create an interchain disulfide bond between the two constant domain cysteine residues, which is not present in unmodified TCRs. Such modified TCRs may form more stable heterodimers. In particular embodiments, the Cα domain comprises a Thr→Cys substitution at position 48 of the wild type protein sequence, and the Cβ domain comprises a Ser→Cys substitution at position 56 of the wild type protein sequence (see, PCT Published Application No. WO2015/184228). An exemplary cysteine modified TCR Cβ constant region comprises an amino acid sequence of SEQ ID NO:93.

In certain embodiments, a TCR comprises substitutions of one, two, or three amino acids in the transmembrane domain of the constant region of one or both of the α and β chains with a hydrophobic amino acid to increase the hydrophobicity of the transmembrane domain. In certain embodiments, one, two, or three of the residues selected from Ser112, Met114, and Gly115 of the TCRα chain are substituted with Gly, Ala, Val, Leu, Ile, Pro, Phe, Met, or Trp. An exemplary cysteine modified, “LVL” substituted TCR Ca region comprises an amino acid sequence of SEQ ID NO:95.

In certain embodiments, the CER and the CAR/or TCR binding protein encoded by the tandem expression cassette target the same antigen. In other embodiments, the CER and the CAR/or TCR binding protein encoded by the tandem expression cassette each target different antigens.

### Polynucleotides, Expression Cassettes, Vectors, and Modified Host Cells

In certain aspects, the present disclosure provides nucleic acid molecules that encode any one or more of the receptors (e.g., CERs, CARs, and TCR binding proteins) described herein. A nucleic acid may refer to a single- or double-stranded DNA, cDNA, or RNA, and may include a positive and a negative strand of the nucleic acid which complement one another, including antisense DNA, cDNA, and RNA. A nucleic acid may be naturally occurring or synthetic forms of DNA or RNA. The nucleic acid sequences encoding a desired receptor can be obtained or produced using recombinant methods known in the art using standard techniques, such as by screening libraries from cells expressing the desired sequence or a portion thereof, by deriving the sequence from a vector known to include the same, or by isolating the sequence or a portion thereof directly from cells or tissues containing the same as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). Alternatively, the sequence of interest can be produced synthetically, rather than being cloned.

Polynucleotides encoding the receptor compositions provided herein may be derived from any animal, such as humans, primates, cows, horses, sheep, dogs, cats, mice, rats, rabbits, guinea pigs, pigs, or a combination thereof. In certain embodiments, a polynucleotide encoding at least one receptor or both receptors contained in the tandem expression cassette is from the same animal species as the host cell into which the polynucleotide is inserted.

In certain embodiments, a polynucleotide encoding a receptor comprises a sequence encoding a signal peptide (also referred to as leader peptide or signal sequence) at the 5′-end for targeting of the precursor protein to the secretory pathway. The signal peptide is optionally cleaved from the N-terminus of the extracellular domain during cellular processing and localization of the receptor to the host cell membrane. A polypeptide from which a signal peptide sequence has been cleaved or removed may also be called a mature polypeptide. Examples of signal peptides that may be used in the receptors of the present disclosure include signal peptides derived from endogenous secreted proteins, including, e.g., GM-CSF (amino acid sequence of SEQ ID NO:64) or Tim4 (amino acid sequence of SEQ ID NO:65). As used herein, reference to a polynucleotide or polypeptide sequence of a receptor, e.g., CER, CAR, or TCR binding protein, provided herein may include or exclude the signal sequence. It is understood by persons of skill in the art that for sequences disclosed herein that include a signal peptide sequence, the signal peptide sequence may be replaced with another signal peptide that is capable of trafficking the encoded protein to the extracellular membrane.

In certain embodiments, a receptor encoding polynucleotide of the present disclosure is codon optimized for efficient expression in a target host cell comprising the polynucleotide (see, e.g, Scholten et al., Clin. Immunol. 119:135-145 (2006)). As used herein, a “codon optimized” polynucleotide comprises a heterologous polynucleotide having codons modified with silent mutations corresponding to the abundances of tRNA in a host cell of interest.

Polynucleotides encoding at least two transgenes (e.g., a CER and CAR, a CER and TCR binding protein) provided in the present disclosure may be used to compose tandem expression cassettes. A tandem expression cassette refers to a component of a vector nucleic acid comprising at least two transgenes under the control of, or operatively linked to, the same set of regulatory sequences for tandem or co-expression of the at least two transgenes. Regulatory sequences that may be used in tandem expression cassettes of the present disclosure include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; sequences that enhance protein secretion, or any combination thereof.

In certain embodiments, a tandem expression cassette can be constructed to optimize spatial and temporal control. For example, a tandem expression cassette can include promoter elements to optimize spatial and temporal control. In some embodiments, a tandem expression cassette includes tissue specific promoters or enhancers that enable specific induction of a tandem expression cassette to an organ, a cell type (e.g., immune cell), or a pathologic microenvironment, such as a tumor or infected tissue. An “enhancer” is an additional promoter element that can function either cooperatively or independently to activate transcription. In certain embodiments, a tandem expression cassette includes a constitutive promoter. An exemplary constitutive promoter for use in tandem expression cassettes of the present disclosure is an EF-1α promoter. In certain embodiments, a tandem expression cassette includes an inducible promoter. In certain embodiments, a tandem expression cassette includes a tissue specific promoter.

The at least two transgenes contained within the tandem expression cassettes may be in any order. For example, a tandem expression cassette comprising a polynucleotide a CER and a polynucleotide encoding a CAR may be arranged from 5′ to 3′ : CER-CAR, or CAR-CER. In another example, a tandem expression cassette comprising a polynucleotide a CER and a polynucleotide encoding a TCR may be arranged from 5′ to 3′: CER-TCR, or TCR-CER.

In certain embodiments, receptors that comprise two or more polypeptide chains that associate to form a multimer or complex may be encoded by two or more polynucleotide molecules within a tandem expression construct. Exemplary multimeric receptors contemplated for expression in tandem expression constructs of the present disclosure include multichain CARs, TCRs, TCR-CARs, and TRuC™ constructs. Accordingly, exemplary tandem expression cassette embodiments encoding a CER and a TCR may comprise a polynucleotide encoding a CER, a polynucleotide encoding a TCRα chain polypeptide, and a polynucleotide encoding a TCRβ chain polypeptide.

In certain embodiments, tandem expression cassettes of the present disclosure may comprise an internal ribosome entry site (IRES) or peptide cleavage site such as a furin cleavage site or viral 2A peptide, disposed between each polynucleotide contained within the tandem expression cassette to allow for coexpression of multiple proteins from a single mRNA. For example, an IRES, furin cleavage site, or viral 2A peptide may be disposed between a polynucleotide encoding a CER and a polynucleotide encoding a CAR within a tandem expression cassette. In another example, an IRES, furin cleavage site, or viral 2A peptide may be disposed between each of a polynucleotide encoding a CER, a polynucleotide encoding a TCRα chain polypeptide, and a polynucleotide encoding a TCRβ chain polypeptide. In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of any one of SEQ ID NOs:67, 68, 69 and 75. An exemplary P2A peptide comprises an amino acid sequence of SEQ ID NO:70 or 71. An exemplary E2A peptide sequence comprises an amino acid sequence of SEQ ID NO:72. An exemplary F2A peptide sequence comprises an amino acid sequence of SEQ ID NO:73.

Certain embodiments of tandem expression cassettes of the present disclosure comprise a polynucleotide encoding a CAR/or TCR specific for a target antigen (e.g., tumor antigen) and a polynucleotide encoding a CER that binds a pro-engulfment marker (e.g., an apoptosis marker such as phosphatidylserine). Upon binding a target cell expressing the target antigen by the CAR/or TCR, a cell modified to express such a tandem expression cassette induces apoptosis of the target cell. Apoptosis induces exposure of pro-engulfment markers on the target cell, such as phosphatidylserine, which may then target the damaged or apoptotic cells for engulfment by the CER.

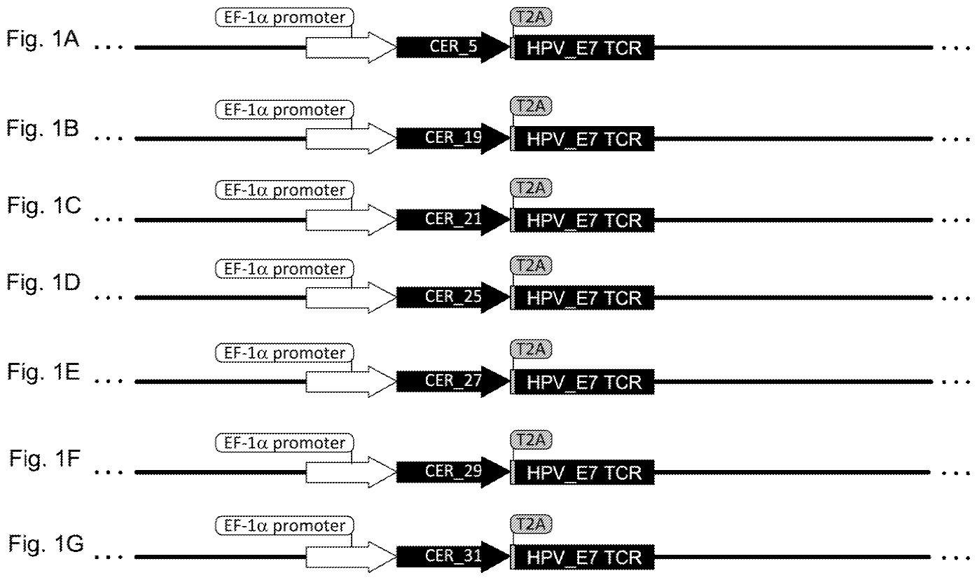

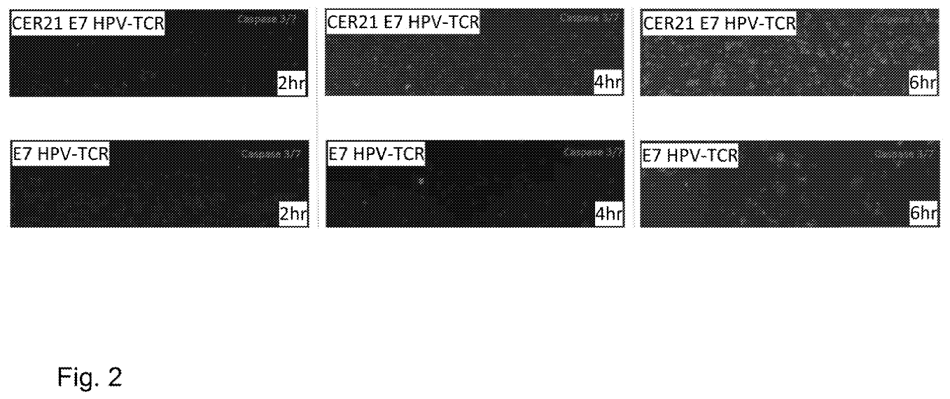

An exemplary tandem expression cassette of the present disclosure comprises: (a) a polynucleotide encoding a CER comprising: an extracellular domain comprising a Tim4 binding domain that binds to phosphatidylserine; a TLR4 engulfment signaling domain; a Tim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1A). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER5-HPV16 E7 TCR”) comprises CER5 comprising an amino acid sequence of SEQ ID NO:97 and HPV16 E7 TCR comprising an amino acid sequence of SEQ ID NO:90.

Another exemplary tandem expression cassette of the present disclosure comprises: (a) a polynucleotide encoding a CER comprising: an extracellular domain comprising a Tim4 binding domain that binds to phosphatidylserine; a TLR5 engulfment signaling domain; a Tim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1B). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER19-HPV16 E7 TCR”) comprises CER19 comprising an amino acid sequence of SEQ ID NO:98 and HPV16 E7 TCR comprising an amino acid sequence of SEQ ID NO:90.

Another exemplary tandem expression cassette of the present disclosure comprises: (a) a polynucleotide encoding a CER comprising: an extracellular domain comprising a Tim4 binding domain; a TLR8 engulfment signaling domain; a Tim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1C). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER21-HPV16 E7 TCR”) comprises CER21 comprising an amino acid sequence of SEQ ID NO:99 and HPV16 E7 TCR comprising an amino acid sequence of SEQ ID NO:90.

Another exemplary tandem expression cassette of the present disclosure comprises: (a) a polynucleotide encoding a CER comprising: an extracellular domain comprising a Tim4 binding domain that binds to phosphatidylserine; a NFAM1 engulfment signaling domain; a Tim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1D). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER25-HPV16 E7 TCR”) comprises CER25 comprising an amino acid sequence of SEQ ID NO:100 and HPV16 E7 TCR comprising an amino acid sequence of SEQ ID NO:90.

Another exemplary tandem expression cassette of the present disclosure comprises: (a) a polynucleotide encoding a CER comprising: an extracellular domain comprising a Tim4 binding domain that binds to phosphatidylserine; a TLR2 engulfment signaling domain; aTim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1E). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER27-HPV16 E7 TCR”) CER27 comprising an amino acid sequence of SEQ ID NO:101 and HPV16 E7 TCR comprising an amino acid sequence of SEQ ID NO:90.

Another exemplary tandem expression cassette of the present disclosure comprises: (a) a polynucleotide encoding a CER comprising: an extracellular domain comprising a Tim4 binding domain that binds to phosphatidylserine; a Traf6 engulfment signaling domain; a Tim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1F). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER29-HPV16 E7 TCR”) comprises CER29 comprising an amino acid sequence of SEQ ID NO:102 and HPV16 E7 TCR comprising an amino acid sequence of SEQ ID NO:90.

Yet another exemplary tandem expression cassette of the present disclosure comprises: (a) a polynucleotide encoding a CER comprising: an extracellular domain comprising a Tim4 binding domain that binds to phosphatidylserine; a Traf3 engulfment signaling domain; a Tim4 transmembrane domain positioned between and connecting the extracellular domain and the engulfment signaling domain; (b) a polynucleotide encoding a HPV-E7 specific recombinant T cell receptor (TCR) beta chain comprising a TCR beta variable region and a TCR beta constant region; and (c) a polynucleotide encoding a HPV-E7 specific recombinant TCR alpha chain comprising a TCR alpha variable region and a TCR alpha constant region (see FIG. 1G). In certain embodiments, the tandem expression cassette comprises a 2A peptide sequence interspersed between the polynucleotide encoding the CER, the polynucleotide encoding the TCRβ chain, and the polynucleotide encoding the TCRα chain. In certain embodiments, the tandem expression cassette comprises an EF-1α promoter operably linked to the CER and TCR polynucleotides. In certain embodiments, such an exemplary tandem expression cassette (“CER31-HPV16 E7 TCR”) comprises CER31 comprising an amino acid sequence of SEQ ID NO:103 and HPV16 E7 TCR comprising an amino acid sequence of SEQ ID NO:90.

A polynucleotide encoding a desired tandem expression cassette can be inserted into an appropriate vector, e.g., a viral vector, non-viral plasmid vector, and non-viral vectors, such as lipid-based DNA vectors, modified mRNA (modRNA), self-amplifying mRNA, CELiD, and transposon-mediated gene transfer (PiggyBac, Sleeping Beauty), for introduction into a host cell of interest (e.g., an immune cell). Polynucleotides encoding a tandem expression cassette of the present disclosure can be cloned into any suitable vector, such as an expression vector, a replication vector, a probe generation vector, or a sequencing vector. In certain embodiments, a polynucleotide encoding the CER, and a polynucleotide encoding the CAR or TCR binding protein are joined together into a single polynucleotide and then inserted into a vector. In other embodiments, a polynucleotide encoding the CER, and a polynucleotide encoding the CAR or TCR binding protein may be inserted separately into a vector such that the expressed amino acid sequence produces a functional CER and CAR/or TCR. A vector that encodes a tandem expression cassette is referred to herein as a “tandem expression vector.”

In certain embodiments, a vector comprises a polynucleotide encoding a tandem expression cassette . In certain embodiments, a vector comprises a tandem expression cassette encoding a CER and a CAR/or TCR binding protein.

In certain embodiments, vectors that allow long-term integration of a tandem expression cassette and propagation to daughter cells are utilized. Examples include viral vectors such as, adenovirus, adeno-associated virus, vaccinia virus, herpes viruses, cytomegalovirus, pox virus, or retroviral vectors, such as lentiviral vectors. Vectors derived from lentivirus can be used to achieve long-term gene transfer and have added advantages over vectors including the ability to transduce non-proliferating cells, such as hepatocytes, and low immunogenicity.

In certain embodiments, non-integrating vectors that that remain episomal are used for the tandem expression cassettes of the present disclosure. Examples of non-integrating viral vectors include adenoviral vectors and integrating viral vectors that have been mutated to be non-integrating, such as non-integrating lentiviral vectors and non-integrating foamy virus vectors.

A vector that encodes a core virus is referred to herein as a “viral vector.” There are a large number of available viral vectors suitable for use with the compositions of the instant disclosure, including those identified for human gene therapy applications (see Pfeifer and Verma, Ann. Rev. Genomics Hum. Genet. 2:177, 2001). Suitable viral vectors include vectors based on RNA viruses, such as retrovirus-derived vectors, e.g., Moloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric receptor transgenes are known in the art and have been previous described, for example, in U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; Verhoeyen et al., Methods Mol. Biol. 506:97, 2009. Retroviral and lentiviral vector constructs and expression systems are also commercially available.

In certain embodiments, a viral vector is used to introduce a non-endogenous polynucleotide encoding a tandem expression cassette to a host cell. A viral vector may be a retroviral vector or a lentiviral vector. A viral vector may also include a nucleic acid sequence encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising a fluorescent protein (e.g., green, yellow), an extracellular domain of human CD2, or a truncated human EGFR (EGFRt or tEGFR; see Wang et al., Blood 118:1255, 2011). An exemplary tEGFR sequence comprises an amino acid sequence of SEQ ID NO:82.

Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5: 1517, 1998).

Other viral vectors recently developed for gene therapy uses can also be used with the compositions and methods of this disclosure. Such vectors include those derived from baculoviruses and a-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

Where temporal control is desired, a tandem expression cassette vector may include an element that allows for inducible depletion of transduced cells. For example, such a vector may include an inducible suicide gene. A suicide gene may be an apoptotic gene or a gene that confers sensitivity to an agent (e.g., a drug). Exemplary suicide genes include chemically inducible caspase 9 (iCASP9) (U.S. Patent Publication No. 2013/0071414), chemically inducible Fas, or Herpes simplex virus thymidine kinase (HSV-TK), which confers sensitivity to ganciclovir. In further embodiments, a tandem expression cassette vector can be designed to express a known cell surface antigen that, upon infusion of an associated antibody, enables depletion of transduced cells. Examples of cell surface antigens and their associated antibodies that may be used for depletion of transduced cells include CD20 and Rituximab, RQR8 (combined CD34 and CD20 epitopes, allowing CD34 selection and anti-CD20 deletion) and Rituximab, and EGFR and Cetuximab.

Inducible vector systems, such as the tetracycline (Tet)-On vector system which activates transgene expression with doxycycline (Heinz et al., Hum. Gene Ther. 2011, 22:166-76) may also be used for inducible expression of tandem expression cassettes. Small molecule responsive transcription factors may also be used to regulate expression. Inducible expression of tandem expression cassettes may be also accomplished via retention using a selective hook (RUSH) system based on streptavidin anchored to the membrane of the endoplasmic reticulum through a hook and a streptavidin binding protein introduced into the CER and CAR/or TCR structure, where addition of biotin to the system leads to the release of the CER and CAR/or TCR from the endoplasmic reticulum (Agaugue et al., 2015, Mol. Ther. 23(Suppl. 1):588).

In certain embodiments, a tandem expression cassette modified host cell may also be modified to co-express one or more small GTPases. Rho GTPases, a family of small (˜21 k Da) signaling G proteins and also a subfamily of the Ras superfamily, regulate actin cytoskeleton organization in various cell types and promote pseudopod extension and phagosome closure during phagocytosis (see, e.g., Castellano et al., 2000, J. Cell Sci. 113:2955-2961). Engulfment requires F-actin recruitment beneath tethered cells or particles, and F-actin rearrangement to allow membrane extension resulting in cell or particle internalization. RhoGTPases include RhoA, Rac1, Rac2, RhoG, and CDC42. Other small GTPases, such as Rapl, is involved in regulation of complement mediated phagocytosis. Co-expression of a small GTPase with the tandem expression cassette encoding a CER may promote or enhance target cell or particle internalization and/or phagosome formation by the host cell. In some embodiments, a recombinant nucleic acid molecule encoding a GTPase is encoded on a separate vector than the tandem expression cassette-containing vector. In other embodiments, a recombinant nucleic acid molecule encoding a GTPase is encoded on the same vector as the tandem expression cassette. The GTPase and tandem expression may be expressed under the regulation of different promoters on the same vector (e.g., at different multiple cloning sites). Alternatively, the tandem expression cassette and GTPase may be expressed under the regulation of one promoter in a multicistronic vector.

Examples of GTPases that may be co-expressed with a tandem expression cassette include Rac1, Rac2, Rab5 (also referred to as Rab5a), Rab7, Rap1, RhoA, RhoG, CDC42, or any combination thereof. In specific embodiments, the GTPase comprises or is a sequence that is at least 90%, at least 91%, at least 92%, at least 93%, at least 94%, at least 95%, at least 96%, at least 97%, at least 98%, at least 99%, at least 99.5%, or 100% identical to a Rac1 amino acid sequence of SEQ ID NO:83, a Rab5 amino acid sequence of SEQ ID NO:84, a Rab7 amino acid sequence of SEQ ID NO:85, a Rapl amino acid sequence of SEQ ID NO:86, a RhoA amino acid sequence of SEQ ID NO:87, a CDC42 amino acid sequence of SEQ ID NO:108, or any combination thereof. In certain embodiments, expression of the GTPase is induced or regulated in a host cell such that following a sufficient amount of time for the CER encoded by the tandem expression cassette to have bound its target antigen, the expression of GPTase is switched on. In further embodiments, expression of the GTPase may be switched off following a sufficient amount of time for CER mediated engulfment of the cells expressing the target antigen.

In certain embodiments, a cell, such as an immune cell, obtained from a subject may be genetically modified into a non-natural or recombinant cell (e.g., a non-natural or recombinant immune cell) by introducing a tandem expression cassette as described herein and whereby the cell expresses a cell surface localized CER and CAR/or TCR. In certain embodiments, a host cell is an immune cell, such as a myeloid progenitor cell or a lymphoid progenitor cell. Exemplary immune cells that may be modified to comprise a tandem expression cassette or a vector comprising a tandem expression cassette include a T cell, a natural killer cell, a B cell, a lymphoid precursor cell, an antigen presenting cell, a dendritic cell, a Langerhans cell, a myeloid precursor cell, a mature myeloid cell, a monocyte, or a macrophage.

In certain embodiments, a B cell is genetically modified to express a tandem expression cassette of the present disclosure. B cells possess certain properties that may be advantageous as host cells, including: trafficking to sites of inflammation , capable of internalizing and presenting antigen, capable of costimulating T cells, highly proliferative, and self-renewing (persist for life). In certain embodiments, a tandem expression cassette modified B cell is capable of digesting an engulfed target cell or engulfed target particle into smaller peptides and presenting them to T cells via an MHC molecule. Antigen presentation by a tandem expression cassette modified B cell may contribute to antigen spreading of the immune response to non-targeted antigens. B cells include progenitor or precursor cells committed to the B cell lineage (e.g., pre-pro-B cells, pro-B cells, and pre-B cells); immature and inactivated B cells; or mature and functional or activated B cells. In certain embodiments, B cells may be naive B cells, plasma cells, regulatory B cells, marginal zone B cells, follicular B cells, lymphoplasmacytoid cell, plasmablast cell, memory B cells, or any combination thereof. Memory B cells may be distinguished from naive B cells by expression of CD27, which is absent on naive B cells. In certain embodiments, the B cells can be primary cells or cell lines derived from human, mouse, rat, or other mammals. B cell lines are well known in the art. If obtained from a mammal, a B cell can be obtained from numerous sources, including blood, bone marrow, spleen, lymph node, or other tissues or fluids. A B cell composition may be enriched or purified.

In certain embodiments, a T cell is genetically modified to express a tandem expression cassette of the present disclosure. Exemplary T cells include CD4⁺ helper, CD8⁺ effector (cytotoxic), naïve (CD45 RA+, CCR7+, CD62L+, CD27+, CD45RO−), central memory (CD45RO⁺, CD62L⁺, CD8⁺), effector memory (CD45RA+, CD45RO−, CCR7−, CD62L−, CD27−), T memory stem, regulatory, mucosal-associated invariant (MAIT), γδ (gd), tissue resident T cells, natural killer T cells, or any combination thereof. In certain embodiments, the T cells can be primary cells or cell lines derived from human, mouse, rat, or other mammals. If obtained from a mammal, a T cell can be obtained from numerous sources, including blood, bone marrow, lymph node, thymus, or other tissues or fluids. A T cell composition may be enriched or purified. T cell lines are well known in the art, some of which are described in Sandberg et al., Leukemia 21:230, 2000. In certain embodiments, the T cells lack endogenous expression of a TCRα gene, TCRβ gene, or both. Such T cells may naturally lack endogenous expression of TCRα and β chains, or may have been modified to block expression (e.g., T cells from a transgenic mouse that does not express TCR α and β chains or cells that have been manipulated to inhibit expression of TCR α and β chains) or to knockout a TCRα chain, a TCRβ chain, or both genes.

In certain embodiments, host cells expressing a tandem expression cassette of the present disclosure are not T cells or cells of a T cell lineage, but cells that are progenitor cells, stem cells or cells that have been modified to express cell surface anti-CD3.

In certain embodiments, gene editing methods are used to modify the host cell genome to comprise a tandem expression cassette of the present disclosure. Gene editing, or genome editing, is a method of genetic engineering wherein DNA is inserted, replaced, or removed from a host cell's genome using genetically engineered endonucleases. The nucleases create specific double-stranded breaks at targeted loci in the genome. The host cell's endogenous DNA repair pathways then repair the induced break(s), e.g., by non-homologous ending joining (NHEJ) and homologous recombination. Exemplary endonucleases useful for gene editing include a zinc finger nuclease (ZFN), a transcription activator-like effector (TALE) nuclease, a clustered regularly interspaced short palindromic repeats (CRISPR)/Cas nuclease system (e.g., CRISPR-Cas9), a meganuclease, or combinations thereof. Methods of disrupting genes or gene expression in immune cells including B cells and T cells, using gene editing endonucleases are known in the art and described, for example, in PCT Publication Nos. WO 2015/066262; WO 2013/074916; WO 2014/059173; Cheong et al., Nat. Comm. 2016 7:10934; Chu et al., Proc. Natl. Acad. Sci. USA 2016 113:12514-12519; methods from each of which are incorporated herein by reference in their entirety.

In certain embodiments, expression of an endogenous gene of the host cell is inhibited, knocked down, or knocked out. Examples of endogenous genes that may be inhibited, knocked down, or knocked out in a B cell include IGH, IGκ, IGλ, or any combination thereof. Examples of endogenous genes that may be inhibited, knocked down, or knocked out in a T cell include a TCR gene (TRA or TRB), an HLA gene (HLA class I gene or HLA class II gene), an immune checkpoint molecule (PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, or PVRIG/CD112R), or any combination thereof. Expression of an endogenous gene may be inhibited, knocked down, or knocked out at the gene level, transcriptional level, translational level, or a combination thereof. Methods of inhibiting, knocking down, or knocking out an endogenous gene may be accomplished, for example, by an RNA interference agent (e.g., siRNA, shRNA, miRNA, etc.) or an engineered endonuclease (e.g., CRISPR/Cas nuclease system, a zinc finger nuclease (ZFN), a Transcription Activator Like Effector nuclease (TALEN), a meganuclease), or any combination thereof. In certain embodiments, an endogenous B cell gene (e.g., IGH, IGκ, or IGλ) is knocked out by insertion of a tandem expression cassette of the present disclosure into the locus of the endogenous B cell gene, such as via an engineered endonuclease. In certain embodiments, an endogenous T cell gene (e.g., a TCR gene, an HLA gene, or an immune checkpoint molecule gene) is knocked out by insertion of a polynucleotide encoding a tandem expression cassette of the present disclosure into the locus of the endogenous T cell gene, such as via an engineered endonuclease.

The present disclosure also provides a composition comprising a population of tandem expression cassette modified host cells. In certain embodiments, the population of tandem expression cassette modified host cells may be a population of B cells, a population of T cells, a population of natural killer cells, a population of lymphoid precursor cells, a population of antigen presenting cells, a population of dendritic cells, a population of Langerhans cells, a population of myeloid precursor cells, a population of mature myeloid cells, or any combination thereof. Furthermore, a population of tandem expression cassette modified host cells of a particular cell type may be composed of one or more subtypes. For example, a population of B cells may be composed of tandem expression cassette modified naïve B cells, plasma cells, regulatory B cells, marginal zone B cells, follicular B cells, lymphoplasmacytoid cell, plasmablast cell, memory B cells, or any combination thereof In another example, a population of T cells may be composed of tandem expression cassette modified CD4⁺ helper T cells, CD8⁺ effector (cytotoxic) T cells, naïve (CD45 RA+, CCR7+, CD62L+, CD27+, CD45RO−) T cells, central memory (CD45RO⁺, CD62L⁺, CD8⁺) T cells, effector memory (CD45RA+, CD45RO−, CCR7−, CD62L−, CD27−) T cells, T memory stem cells, regulatory T cells, mucosal-associated invariant T cells (MAIT), γδ (gd), tissue resident T cells, natural killer T cells, or any combination thereof.

In certain embodiments, a population of host cells is composed of cells that each express the same tandem expression cassette. In other embodiments, a population of host cells is composed of a mixture of two or more subpopulation of host cells, wherein each subpopulation expresses a different tandem expression cassette.

In certain embodiments, when preparing tandem expression cassette modified host cells, e.g., B cells or T cells, one or more growth factor cytokines that promote proliferation of the host cells, e.g., B cells or T cells, may be added to the cell culture. The cytokines may be human or non-human. Exemplary growth factor cytokines that may be used to promote T cell proliferation include IL-2, IL-15, or the like. Exemplary growth factor cytokines that may be used to promote B cell proliferation include CD4OL, IL-2, IL-4, IL-15, IL-21, BAFF, or the like.

Prior to genetic modification of the host cells with a tandem expression cassette vector, a source of host cells (e.g., T cells, B cells, natural killer cells, etc.) is obtained from a subject (e.g., whole blood, peripheral blood mononuclear cells, bone marrow, lymph node tissue, cord blood, thymus tissue, tissue from a site of infection, ascites, pleural effusion, spleen tissue), from which host cells are isolated using methods known in the art. Specific host cell subsets can be collected in accordance with known techniques and enriched or depleted by known techniques, such as affinity binding to antibodies, flow cytometry and/or immunomagnetic selection. After enrichment and/or depletion steps and introduction of a tandem expression cassette, in vitro expansion of the desired modified host cells can be carried out in accordance with known techniques, or variations thereof that will be apparent those skilled in the art.

The expression of receptors encoded by tandem expression cassettes on host cells may be functionally characterized according to any of a large number of art-accepted methodologies for assaying host cell (e.g., T cell) activity, including determination of T cell binding, activation or induction and also including determination of T cell responses that are antigen-specific. Examples include determination of T cell proliferation, T cell cytokine release, antigen-specific T cell stimulation, CTL activity (e.g., by detecting ⁵¹Cr or Europium release from pre-loaded target cells, induction of caspase activity in target cells, extracellular release of lactate dehydrogenase by target cells), changes in T cell phenotypic marker expression, and other measures of T cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein. Cytokine levels may be determined according to methods known in the art, including for example, ELISA, ELISPOT, intracellular cytokine staining, flow cytometry, and any combination thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like.

In certain embodiments, a tandem expression cassette modified host cell comprising a CER has a phagocytic index of about 20 to about 1,500 for a target cell. A “phagocytic index” is a measure of phagocytic activity of the transduced host cell as determined by counting the number of target cells or particles ingested per tandem expression cassette modified host cell during a set period of incubation of a suspension of target cells or particles and tandem expression cassette modified host cells in media. Phagocytic index may be calculated by multiplying [total number of engulfed target cells/total number of counted tandem expression cassette modified cells (e.g., phagocytic frequency)]×[average area of target cell or particle staining per tandem expression cassette⁺ host cell×100 (e.g., hybrid capture)] or [total number of engulfed particles/total number of counted tandem expression cassette modified host cells]×[number of tandem expression cassette modified host cells containing engulfed particles/total number of counted tandem expression cassette⁺ cells]×100. In certain embodiments, a tandem expression cassette modified cell has a phagocytic index of about 30 to about 1,500; about 40 to about 1,500; about 50 to about 1,500; about 75 to about 1,500; about 100 to about 1,500; about 200 to about 1,500; about 300 to about 1,500; about 400 to about 1,500; about 500 to about 1,500; about 20 to about 1,400; about 30 to about 1,400; about 40 to about 1,400; about 50 to about 1,400; about 100 to about 1,400; about 200 to about 1,400; about 300 to about 1,400; about 400 to about 1,400; about 500 to about 1,400; about 20 to about 1,300; about 30 to about 1,300; about 40 to about 1,300; about 50 to about 1,300; about 100 to about 1,300; about 200 to about 1,300; about 300 to about 1,300; about 400 to about 1,300; about 500 to about 1,300; about 20 to about 1,200; about 30 to about 1,200; about 40 to about 1,200; about 50 to about 1,200; about 100 to about 1,200; about 200 to about 1,200; about 300 to about 1,200; about 400 to about 1,200; about 500 to about 1,200; about 20 to about 1,100; about 30 to about 1,100; about 40 to about 1,100; about 50 to about 1,100; about 100 to about 1,100; about 200 to about 1,100; about 300 to about 1,100; about 400 to about 1,100; or about 500 to about 1,100; about 20 to about 1,000; about 30 to about 1,000; about 40 to about 1,000; about 50 to about 1,000; about 100 to about 1,000; about 200 to about 1,000; about 300 to about 1,000; about 400 to about 1,000; or about 500 to about 1,000; about 20 to about 750; about 30 to about 750; about 40 to about 750; about 50 to about 750; about 100 to about 750; about 200 to about 750; about 300 to about 750; about 400 to about 750; or about 500 to about 750; about 20 to about 500; about 30 to about 500; about 40 to about 500; about 50 to about 500; about 100 to about 500; about 200 to about 500; or about 300 to about 500. In further embodiments, the incubation time is from about 2 hours to about 4 hours, e.g., about 2 hours, about 3 hours, or about 4 hours. In yet further embodiments, a tandem expression cassette modified cell exhibits phagocytic index that is statistically significantly higher than a control cell transduced with truncated EGFR. Phagocytic index may be calculated using methods known in the art and as further described in the Examples and PCT Application No. PCT/US2017/053553 (incorporated herein by reference in its entirety), including quantification by flow cytometry or fluorescence microscopy.

Host cells may be from an animal, such as a human, primate, cow, horse, sheep, dog, cat, mouse, rat, rabbit, guinea pig, pig, or a combination thereof. In a preferred embodiment, the animal is a human. Host cells may be obtained from a healthy subject or a subject having a disease associated with expression or overexpression of an antigen.

### Methods of Use

In one aspect, the present disclosure provides methods for conferring antigen specific cytolytic activity and engulfment activity to a cell comprising introducing into a host cell a tandem expression cassette according to any of the embodiments described herein; and expressing the CER and CAR/or TCR binding protein in the host cell. In certain embodiments, the CER and CAR/or TCR binding protein bind to the same target antigen. In some embodiments, the CER and CAR/or TCR bind to different target antigens. In certain embodiments, host cells modified with tandem expression cassettes of the present disclosure are capable of engulfing target cells or portions of target cells. Thus, cells modified with tandem expression cassettes of the present disclosure may possess targeted cell killing capacity via multiple modalities: cytolysis of the target cell, engulfment of the whole target cell, engulfment of parts of the target cell, or any combination thereof.

In another aspect, the present disclosure provides methods for enhancing antigen-specific cytotoxic activity of a cell comprising introducing into a host cell a tandem expression cassette according to any of the embodiments described herein; and expressing the CER and CAR/or TCR binding protein in the host cell, wherein the expression of the tandem expression cassette enhances cytotoxic activity of the host cell as compared to a host cell that expresses the CAR/or TCR binding protein alone. In certain embodiments, the cytotoxic activity of the host cell is increased at least about 10%, 15%, 20%, 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more as compared to the host cell expressing the CAR/or TCR alone. In further embodiments, the tandem expression cassette confers a synergistic cytotoxic response. In some embodiments, the host cell is a T cell or an NK cell. Methods of measuring cytotoxic activity of host cells, particularly immune cells such as T cells and NK cells, include a chromium (⁵¹Cr)-release assay, a β-gal or firefly luciferase release assay, flow cytometric methods of measuring target cell death and effector cell activity (see, e.g., Expert Rev. Vaccines, 2010, 9:601-616). In certain embodiments, cytotoxic activity of host cells may be measured by detecting target cell apoptosis following exposure to the host cell, e.g., caspase 3/7 activity, lactate dehydrogenase release.

In another aspect, a tandem expression cassette according to any of the embodiments provided herein may be used in a method of treating a subject suffering from a disease, disorder or undesired condition. Embodiments of these methods include administering to a subject a therapeutically effective amount of a pharmaceutical composition including one or more tandem expression cassettes, polynucleotides encoding one or more tandem expression cassettes, vectors comprising one or more tandem expression cassettes, or a population of host cells genetically modified to express one or more tandem expression cassettes according to the present description.

Diseases that may be treated with cells expressing tandem expression cassette provided in the present disclosure include cancer, autoimmune diseases, and infectious diseases (viral, bacterial, fungal, protozoan infections). Adoptive immune and gene therapies are promising treatments for various types of cancer (Morgan et al., Science 314:126, 2006; Schmitt et al., Hum. Gene Ther. 20:1240, 2009; June, J. Clin. Invest. 117:1466, 2007) and infectious disease (Kitchen et al., PLoS One 4:38208, 2009; Rossi et al., Nat. Biotechnol. 25:1444, 2007; Zhang et al., PLoS Pathog. 6:e1001018, 2010; Luo et al., J. Mol. Med. 89:903, 2011).

A wide variety of cancers, including solid tumors and leukemias are amenable to treatment using the tandem expression cassette compositions provided herein. Exemplary cancers that may be treated using the receptors, modified host cells, and composition described herein include adenocarcinoma of the breast, prostate, and colon; all forms of bronchogenic carcinoma of the lung; myeloid leukemia; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell). Additional types of cancers that may be treated using the receptors, modified host cells, and composition described herein include histiocytic disorders; malignant histiocytosis; leukemia; Hodgkin's disease; immunoproliferative small; non-Hodgkin's lymphoma; plasmacytoma; multiple myeloma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment using the receptors, modified host cells, and composition described herein: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. The types of cancers that may be treated also include angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Examples of hyperproliferative disorders amenable to therapy using the tandem expression cassette compositions described herein include B-cell cancers, including B-cell lymphomas (such as various forms of Hodgkin's disease, non-Hodgkins lymphoma (NHL) or central nervous system lymphomas), leukemias (such as acute lymphoblastic leukemia (ALL), chronic lymphocytic leukemia (CLL), Hairy cell leukemia, B cell blast transformation of chronic myeloid leukemia) and myelomas (such as multiple myeloma). Additional B cell cancers that may be treated using the receptors, modified host cells, and composition described herein include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

Infectious diseases include those associated with infectious agents and include any of a variety of bacteria (e.g., pathogenic E. coli, S. typhimurium, P. aeruginosa, B. anthracis, C. botulinum, C. difficile, C. perfringens, H. pylori, V. cholerae, Listeria spp., Rickettsia spp., Chlamydia spp., and the like), mycobacteria, and parasites (including any known parasitic member of the Protozoa). Infectious viruses include eukaryotic viruses, such as adenovirus, bunyavirus, herpesvirus, papovavirus, papillomavirus (e.g., HPV), paramyxovirus, picornavirus, rhabdovirus (e.g., Rabies), orthomyxovirus (e.g., influenza), poxvirus (e.g., Vaccinia), reovirus, retrovirus, lentivirus (e.g., HIV), flavivirus (e.g., HCV, HBV) or the like. In certain embodiments, a composition comprising a tandem expression cassette according to the present disclosure is used for treating infection with a microbe capable of establishing a persistent infection in a subject.

Methods of treating a subject comprise administering an effective amount of tandem expression cassette modified cells (i.e., recombinant cells that express tandem expression cassettes). The tandem expression cassette modified cells may be xenogeneic, syngeneic, allogeneic, or autologous to the subject.

Pharmaceutical compositions including tandem expression cassette modified cells may be administered in a manner appropriate to the disease or condition to be treated (or prevented) as determined by persons skilled in the medical art. An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently. The present disclosure provides pharmaceutical compositions comprising tandem expression cassette modified cells and a pharmaceutically acceptable carrier, diluent, or excipient. Suitable excipients include water, saline, dextrose, glycerol, or the like and combinations thereof. Other suitable infusion medium can be any isotonic medium formulation, including saline, Normosol R (Abbott), Plasma-Lyte A (Baxter), 5% dextrose in water, or Ringer's lactate.

A treatment effective amount of cells in a pharmaceutical composition is at least one cell (for example, one tandem expression cassette modified T cell) or is more typically greater than 10² cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m², preferably in a range of about 10⁷ to about 10⁹ cells/m². In a specific embodiment, the tandem expression cassette modified cells are administered in an amount of at least about 1×10⁶ cells, 2×10⁶ cells, 3×10⁶ cells, 4×10⁶ cells, 5×10⁶ cells, 6×10⁶ cells, 7×10⁶ cells, 8×10⁶ cells, 9×10⁶ cells, 1×10⁷ cells, 2×10⁷ cells, 3×10⁷ cells, 4×10⁷ cells, 5×10⁷ cells, 6×10⁷ cells, 7×10⁷ cells, 8×10⁷ cells, 9×10⁷ cells, 1×10⁸ cells, 2×10⁸ cells, 3×10⁸ cells, 4×10⁸ cells, 5×10⁸ cells, 6×10⁸ cells, 7×10⁸ cells, 8×10⁸ cells, 9×10⁸ cells, 1×10⁹ cells, 2×10⁹ cells, 3×10⁹ cells, 4×10⁹ cells, 5×10⁹ cells, 6×10⁹ cells, 7×10⁹ cells, 8×10⁹ cells, 9×10⁹ cells, 1×10¹⁰ cells, 2×10¹⁰ cells, 3×10¹⁰ cells, 4×10¹⁰ cells, 5×10¹⁰ cells, 6×10¹⁰ cells, 7×10¹⁰ cells, 8×10¹⁰ cells, 9×10¹⁰ cells, 1×10¹¹ cells, 2×10¹¹ cells, 3×10¹¹ cells, 4×10¹¹ cells, 5×10¹¹ cells, 6×10¹¹ cells 7×10¹¹ cells, 8×10¹¹ cells, or 9×10¹¹ cells. The number of cells will depend upon the ultimate use for which the composition is intended as well the type of cells included therein. For example, a composition comprising cells modified to contain a tandem expression cassette will comprise a cell population containing from about 5% to about 95% or more of such cells. In certain embodiments, a composition comprising tandem expression cassette modified cells comprises a cell population comprising at least 5%, 10%, 15%, 20%, 25%, 3 0%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 10⁴ cells/ml and generally is greater than 10⁷ cells/ml, generally 10⁸ cells/ml or greater. The cells may be administered as a single infusion or in multiple infusions over a range of time. Repeated infusions of tandem expression cassette modified cells may be separated by days, weeks, months, or even years if relapses of disease or disease activity are present. A clinically relevant number of immune cells can be apportioned into multiple infusions that cumulatively equal or exceed 10⁶, 10⁷, 10⁸, 10⁹, 10¹⁰, or 10¹¹ cells. A preferred dose for administration of a host cell comprising a recombinant expression vector as described herein is about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

Compositions comprising tandem expression cassettes, vectors comprising tandem expression cassettes, or tandem expression cassette modified cells as described herein may be administered intravenously, intraperitoneally, intranasally, intratumorly, into the bone marrow, into the lymph node, and /or into cerebrospinal fluid.

Tandem expression cassette compositions may be administered to a subject in combination with one or more additional therapeutic agents. Examples of therapeutic agents that may be administered in combination with a tandem expression cassette compositions according to the present description include radiation therapy, genetically engineered cellular immunotherapy (e.g., T cell, dendritic cell, natural killer cell, macrophage, chimeric antigen receptor therapy), antibody therapy, immune checkpoint molecule inhibitor therapy, UV light therapy, electric pulse therapy, high intensity focused ultrasound therapy, oncolytic virus therapy, or a pharmaceutical therapy, such as a chemotherapeutic agent, a therapeutic peptide, a hormone, an aptamer, antibiotic, anti-viral agent, anti-fungal agent, anti-inflammatory agent, a small molecule therapy.

Radiation therapy includes external beam radiation therapy (e.g., conventional external beam radiation therapy, stereotactic radiation, 3-dimensional conformal radiation therapy, intensity-modulated radiation therapy, volumetric modulated arc therapy, particle therapy, proton therapy, and auger therapy), brachytherapy, systemic radioisotope therapy, intraoperative radiotherapy, or any combination thereof.

Exemplary antibodies for use in conjunction with the tandem expression cassette compositions described herein include rituxmab, pertuzumab, trastuzumab, alemtuzumab, Ibritumomab tiuxetan, Brentuximab vedotin, cetuximab, bevacizumab, abciximab, adalimumab, alefacept, basilizimab, belimumab, bezlotoxumab, canakinumab, certolizumab pegol, daclizumab, denosumab, efalizumab, golimumab, olaratumab, palivizumab, panitumumab, and tocilizumab.

Exemplary inhibitors of immune checkpoint molecules that may be for use in conjunction with the tandem expression cassette compositions described herein include checkpoint inhibitors targeting PD-L1, PD-L2, CD80, CD86, B7-H3, B7-H4, HVEM, adenosine, GALS, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof In certain embodiments, an immune checkpoint inhibitor may be an antibody, a peptide, an RNAi agent, or a small molecule. An antibody specific for CTLA-4 may be ipilimumab or tremelimumab. An antibody specific for PD-1 may be pidilizumab, nivolumab, or pembrolizumab. An antibody specific for PD-L1 may be durvalumab, atezolizumab, or avelumab.

A chemotherapeutic includes non-specific cytotoxic agents that inhibit mitosis or cell division, as well as molecularly targeted therapy that blocks the growth and spread of cancer cells by targeting specific molecules that are involved in tumor growth, progression, and metastasis (e.g., oncogenes). Exemplary non-specific chemotherapeutics for use in conjunction with the tandem expression cassette compositions described herein may include an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

Examples of chemotherapeutic agents considered for use in combination therapies contemplated herein include vemurafenib, dabrafenib, trametinib, cobimetinib, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®),fdabra tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), ibrutinib, venetoclax, crizotinib, alectinib, brigatinib, ceritinib, and vinorelbine (Navelbine®).

Exemplary alkylating agents for use in combination therapies contemplated herein include nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents for use in combination therapies contemplated herein include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents for use in combination therapies contemplated herein include carboplatin, cisplatin, oxaliplatin, nedaplatin, picoplatin, satraplatin, phenanthriplatin, and triplatin tetranitrate.

Exemplary molecularly targeted inhibitors for use in conjunction with the tandem expression cassette compositions described herein include small molecules that target molecules involved in cancer cell growth and survival, including for example, hormone antagonists, signal transduction inhibitors, gene expression inhibitors (e.g., translation inhibitors), apoptosis inducers, angiogenesis inhibitors (e.g., a VEGF pathway inhibitor), tyrosine kinase inhibitors (e.g., an EGF/EGFR pathway inhibitor), growth factor inhibitors, GTPase inhibitors, serine/threonine kinase inhibitors, transcription factor inhibitors, inhibitors of driver mutations associated with cancer, B-Raf inhibitors, a MEK inhibitors, mTOR inhibitors, adenosine pathway inhibitors, EGFR inhibitors, PI3K inhibitors, BCL2 inhibitors, VEGFR inhibitors, MET inhibitors, MYC inhibitors, BCR-ABL inhibitors, HER2 inhibitors, H-RAS inhibitors, K-RAS inhibitors, PDGFR inhibitors, ALK inhibitors, ROS1 inhibitors, and BTK inhibitors. In certain embodiments, use of molecularly targeted therapy comprises administering a molecularly targeted therapy specific for the molecular target to a subject identified as having a tumor that possesses the molecular target (e.g., driver oncogene). In certain embodiments, the molecular target has an activating mutation. In certain embodiments, use of tandem expression cassette modified cells in combination with a molecularly targeted inhibitor increases the magnitude of anti-tumor response, the durability of anti-tumor response, or both. In certain embodiments, a lower than typical dose of molecularly targeted therapy is used in combination with tandem expression cassette modified cells.

Exemplary angiogenesis inhibitors for use in conjunction with tandem expression cassette compositions described herein may include, without limitation A6 (Angstrom Pharmaceuticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP 02) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQule), ASA404 (Novartis/Antisoma), Atiprimod (Callisto Pharmaceuticals), ATN-161 (Attenuon), AV-412 (Aveo Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Boehringer Ingelheim Pharmaceuticals), BIBW 2992 (Boehringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharaceuticals), Combretastatin (Oxigene), CP-751,871(Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabuse), E7820 (Eisai Limited), E7080 (Eisai Limited), EMD 121974(Cilengitide) (EMD Pharmaceuticals), ENMD-1198 (EntreMed), ENMD-2076 (EntreMed), Endostar (Simcere), Erbitux (ImClone/Bristol-Myers Squibb), EZN-2208 (Enzon Pharmaceuticals), EZN-2968 (Enzon Pharmaceuticals), GC1008 (Genzyme), Genistein, GSK1363089(Foretinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (MedImmune), MLN518(Tandutinib) (Millennium), Neovastat (AE941/Benefin) (Aeterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230(Pasireotide) (Novartis), Squalamine (Genaera), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exelixis), XL647 (Exelixis), XL784 (Exelixis), XL820 (Exelixis), XL999 (Exelixis), ZD6474 (AstraZeneca), Vorinostat (Merck), and ZSTK474.

Exemplary Vascular Endothelial Growth Factor (VEGF) receptor inhibitors for use in conjunction with the tandem expression cassette compositions described herein may include, but are not limited to, Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

Exemplary EGF pathway inhibitors for use in conjunction with the tandem expression cassette compositions described herein may include, without limitation tyrphostin 46, EKB-569, erlotinib (Tarceva®), gefitinib (Iressa®), erbitux, nimotuzumab, lapatinib (Tykerb®), cetuximab (anti-EGFR mAb), ¹⁸⁸Re-labeled nimotuzumab (anti-EGFR mAb), and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980. Exemplary EGFR antibodies include, but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1). Exemplary Epidermal growth factor receptor (EGFR) inhibitors include, but not limited to, Osimertinib (Tagrisso®), Erlotinib hydrochloride (Tarceva®), Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykerb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(1R)-1-phenylethyl]-7H-Pyrrolo[2,3-d]pyrimidin-4-amine (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); and 4-[4-[[(1R)-1-Phenylethyl]amino]-7H-pyrrolo[2,3-d]pyrimidin-6-yl]-phenol (PKI166, CAS 187724-61-4).

Exemplary mTOR inhibitors for use in conjunction with the tandem expression cassette compositions described herein may include, without limitation, rapamycin (Rapamune®), and analogs and derivatives thereof; SDZ-RAD; Temsirolimus (Torisel®; also known as CCI-779); Ridaforolimus (formally known as deferolimus, (1R,2R,4S)-4-[(2R)-2[(1R,9S,12S,15R,16E,18R,19R,21R,23 S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1).

Exemplary Phosphoinositide 3-kinase (PI3K) inhibitors for use in conjunction with the tandem expression cassette compositions described herein may include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6). Exemplary Protein Kinase B (PKB) or AKT inhibitors include, but are not limited to. 8-[4-(1-Aminocyclobutyl)phenyl]-9-phenyl-1,2,4-triazolo[3,4-f][1,6]naphthyridin-3(2H)-one (MK-2206, CAS 1032349-93-1); Perifosine (KRX0401); 4-Dodecyl-N-1,3,4-thiadiazol-2-yl -benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Tricirbine (6-Amino-4-methyl-8-(β-D-ribofuranosyl)-4H,8H-pyrrolo[4,3,2-de]pyrimido[4,5-c]pyridazine); (αS)-α-[[[5-(3-Methyl-1H-indazol-5-yl)-3-pyridinyl]oxy]methyl]-benzeneethanamine (A674563, CAS 552325-73-2); 4-[(4-Chlorophenyl)methyl]-1-(7H-pyrrolo[2,3-d]pyrimidin-4-yl)-4-piperidinamine (CCT128930, CAS 885499-61-6); 4-(4-Chlorophenyl)-4-[4-(1H pyrazol-4-yl)phenyl]-piperidine (AT7867, CAS 857531-00-1); and Archexin (RX-0201, CAS 663232-27-7).

In certain embodiments, a tyrosine kinase inhibitor used in combination with CER modified cells is an anaplastic lymphoma kinase (ALK) inhibitor. Exemplary ALK inhibitors include crizotinib, ceritinib, alectinib, brigatinib, dalantercept, entrectinib, and lorlatinib.

In certain embodiments where tandem expression cassette modified cells are administered in combination with one or more additional therapies, the one or more additional therapies may be administered at a dose that might otherwise be considered subtherapeutic if administered as a monotherapy. In such embodiments, the tandem expression cassette may provide an additive or synergistic effect such that the one or more additional therapies can be administered at a lower dose. Combination therapy includes administration of a tandem expression cassette composition as described herein before an additional therapy (e.g., 1 day to 30 days or more before the additional therapy), concurrently with an additional therapy (on the same day), or after an additional therapy (e.g., 1 day-30 days or more after the additional therapy). In certain embodiments, the tandem expression cassette modified cells are administered after administration of the one or more additional therapies. In further embodiments, the tandem expression cassette modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after administration of the one or more additional therapies. In still further embodiments, the tandem expression cassette modified cells are administered within 4 weeks, within 3 weeks, within 2 weeks, or within 1 week after administration of the one or more additional therapies. Where the one or more additional therapies involves multiple doses, the tandem expression cassette modified cells may be administered after the initial dose of the one or more additional therapies, after the final dose of the one or more additional therapies, or in between multiple doses of the one or more additional therapies.

In certain embodiments, methods of the present disclosure include a depletion step. A depletion step to remove tandem expression cassette modified cells from the subject may occur after a sufficient amount of time for therapeutic benefit in order to mitigate toxicity to a subject. In such embodiments, a vector comprising the tandem expression cassette may include an inducible suicide gene, such as iCASP9, inducible Fas, or HSV-TK. Similarly, the vector may be designed for expression of a known cell surface antigen such as CD20 or truncated EGFR (SEQ ID NO:82) that facilitates depletion of transduced cells through infusion of an associated monoclonal antibody (mAb), for example, Rituximab for CD20 or Cetuximab for EGFR. Alemtuzumab, which targets CD52 present on the surface of mature lymphocytes, may also be used to deplete transduced B cells, T cells, or natural killer cells.

Subjects that can be treated by the compositions and methods of the present disclosure include animals, such as humans, primates, cows, horses, sheep, dogs, cats, mice, rats, rabbits, guinea pigs, or pigs. The subject may be male or female, and can be any suitable age, including infant, juvenile, adolescent, adult, and geriatric subjects.

### EXAMPLES

### Example 1: Construction of Tandem Expression Cassettes

A polynucleotide comprising the extracellular domain of the phosphatidylserine binding protein Tim4 and Tim4 transmembrane domain was fused to the intracellular signaling domain of TLR4 to create chimeric engulfment receptor “CER5” encoding an amino acid sequence of SEQ ID NO:97. A polynucleotide comprising the extracellular domain of the phosphatidylserine binding protein Tim4 and Tim4 transmembrane domain was fused to the intracellular signaling domain of TLR5 to create chimeric engulfment receptor “CER19” encoding an amino acid sequence of SEQ ID NO:98. A polynucleotide comprising the extracellular domain of the phosphatidylserine binding protein Tim4 was Tim4 transmembrane domain and TLR8 intracellular signaling domain to create chimeric engulfment receptor “CER21” encoding an amino acid sequence of SEQ ID NO:99. A polynucleotide comprising the extracellular domain of the phosphatidylserine binding protein Tim4 and Tim4 transmembrane domain was fused to the intracellular signaling domain of NFAM1 to create chimeric engulfment receptor “CER25” encoding an amino acid sequence of SEQ ID NO:100. A polynucleotide comprising the extracellular domain of the phosphatidylserine binding protein Tim4 and Tim4 transmembrane domain was fused to the intracellular signaling domain of TLR2 to create chimeric engulfment receptor “CER27” encoding an amino acid sequence of SEQ ID NO:101. A polynucleotide comprising the extracellular domain of the phosphatidylserine binding protein Tim4 and Tim4 transmembrane domain was fused to the intracellular signaling domain of Traf6 to create chimeric engulfment receptor “CER29” encoding an amino acid sequence of SEQ ID NO:102. A polynucleotide comprising the extracellular domain of the phosphatidylserine binding protein Tim4 and Tim4 transmembrane domain was fused to the intracellular signaling domain of Traf3 to create chimeric engulfment receptor “CER31” encoding an amino acid sequence of SEQ ID NO:103.

A polynucleotide encoding a TCRβ chain and a polynucleotide encoding a TCRα of an HPV16 E7 specific TCR (see, PCT Publication No. WO2015/184228) were fused using a sequence for P2A self-cleaving peptide therebetween. The TCR Vα domain comprises an amino acid sequence of SEQ ID NO:94, and the TCR Vβ region comprises an amino acid sequence of SEQ ID NO:92. The Cα domain comprises a cysteine substitution and LVL substitutions at positions 12, 14, and 15 and comprises an amino acid sequence of SEQ ID NO:95. The CP also comprises a cysteine substitution and comprises an amino acid sequence of SEQ ID NO:93. The encoded HPV16 E7 specific TCR comprises an amino acid sequence of SEQ ID NO:90. Amino acid sequences for the tandem expression constructs described in this example are provided in Table 2.

TABLE 2
Exemplary Tandem Expression Cassettes

| Name | Amino Acid Sequence | SEQ ID NO: # |
|---|---|---|
| CER5_T2A_HPV16 E7 | MSKGLLLLWLVTELWWLYLTPAASEDTIIGFLGQPVT | SEQ ID NO: 176 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLKFYFHLMLLAGCIKYGRGENIYDAFVIYSSQDE | |
| | DWVRNELVKNLEEGVPPFQLCLHYRDFIPGVAIAANI | |
| | IHEGFHKSRKVIVVVSQHFIQSRWCIFEYEIAQTWQF | |
| | LSSRAGIIFIVLQKVEKTLLRQQVELYRLLSRNTYLE | |
| | WEDSVLGRHIFWRRLRKALLDGKSWNPEGTVGTGCNW | |
| | QEATSILEGGGEGRGSLLTCGDVEENPGPMAPGLLCW | |
| | ALLCLLGAGLVDAGVTQSPTHLIKTRGQQVTLRCSPK | |
| | SGHDTVSWYQQALGQGPQFIFQYYEEEERQRGNFPDR | |
| | FSGHQFPNYSSELNVNALLLGDSALYLCASSLGWRGG | |
| | RYNEQFFGPGTRLTVLEDLRNVTPPKVSLFEPSKAEI | |
| | ANKQKATLVCLARGFFPDHVELSWWVNGKEVHSGVCT | |
| | DPQAYKESNYSYCLSSRLRVSATFWHNPRNHFRCQVQ | |
| | FHGLSEEDKWPEGSPKPVTQNISAEAWGRADCGITSA | |
| | SYQQGVLSATILYEILLGKATLYAVLVSTLVVMAMVK | |
| | RKNSRAKRSGSGATNFSLLKQAGDVEENPGPMWGVFL | |
| | LYVSMKMGGTTGQNIDQPTEMTATEGAIVQINCTYQT | |
| | SGFNGLFWYQQHAGEAPTFLSYNVLDGLEEKGRFSSF | |
| | LSRSKGYSYLLLKELQMKDSASYLCASVDGNNRLAFG | |
| | KGNQVVVIPNIQNPEPAVYQLKDPRSQDSTLCLFTDF | |
| | DSQINVPKTMESGTFITDKCVLDMKAMDSKSNGAIAW | |
| | SNQTSFTCQDIFKETNATYPSSDVPCDATLTEKSFET | |
| | DMNLNFQNLLVIVLRILLLKVAGFNLLMTLRLWSS | |
| CER19_T2A_HPV16 E7 | MSKGLLLLWLVTELWWLYLTPAASEDTIIGFLGQPVT | SEQ ID NO: 177 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLTKFRGFCFICYKTAQRLVFKDHPQGTEPDMYKY | |
| | DAYLCFSSKDFTWVQNALLKHLDTQYSDQNRFNLCFE | |
| | ERDFVPGENRIANIQDAIWNSRKIVCLVSRHFLRDGW | |
| | CLEAFSYAQGRCLSDLNSALIMVVVGSLSQYQLMKHQ | |
| | SIRGFVQKQQYLRWPEDFQDVGWFLHKLSQQILKKEK | |
| | EKKKDNNIPLQTVATISLEGGGEGRGSLLTCGDVEEN | |
| | PGPMAPGLLCWALLCLLGAGLVDAGVTQSPTHLIKTR | |
| | GQQVTLRCSPKSGHDTVSWYQQALGQGPQFIFQYYEE | |
| | EERQRGNFPDRFSGHQFPNYSSELNVNALLLGDSALY | |
| | LCASSLGWRGGRYNEQFFGPGTRLTVLEDLRNVTPPK | |
| | VSLFEPSKAEIANKQKATLVCLARGFFPDHVELSWWV | |
| | NGKEVHSGVCTDPQAYKESNYSYCLSSRLRVSATFWH | |
| | NPRNHFRCQVQFHGLSEEDKWPEGSPKPVTQNISAEA | |
| | WGRADCGITSASYQQGVLSATILYEILLGKATLYAVL | |
| | VSTLVVMAMVKRKNSRAKRSGSGATNFSLLKQAGDVE | |
| | ENPGPMWGVFLLYVSMKMGGTTGQNIDQPTEMTATEG | |
| | AIVQINCTYQTSGFNGLFWYQQHAGEAPTFLSYNVLD | |
| | GLEEKGRFSSFLSRSKGYSYLLLKELQMKDSASYLCA | |
| | SVDGNNRLAFGKGNQVVVIPNIQNPEPAVYQLKDPRS | |
| | QDSTLCLFTDFDSQINVPKTMESGTFITDKCVLDMKA | |
| | MDSKSNGAIAWSNQTSFTCQDIFKETNATYPSSDVPC | |
| | DATLTEKSFETDMNLNFQNLLVIVLRILLLKVAGFNL | |
| | LMTLRLWSS | |
| CER21_T2A_HPV16 E7 | MSKGLLLLWLVTELWWLYLTPAASEDTIIGFLGQPVT | SEQ ID NO: 178 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLHHLFYWDVWFIYNVCLAKVKGYRSLSTSQTFYD | |
| | AYISYDTKDASVTDWVINELRYHLEESRDKNVLLCLE | |
| | ERDWDPGLAIIDNLMQSINQSKKTVFVLTKKYAKSWN | |
| | FKTAFYLALQRLMDENMDVIIFILLEPVLQHSQYLRL | |
| | RQRICKSSILQWPDNPKAEGLFWQTLRNVVLTENDSR | |
| | YNNMYVDSIKQYLEGGGEGRGSLLTCGDVEENPGPMA | |
| | PGLLCWALLCLLGAGLVDAGVTQSPTHLIKTRGQQVT | |
| | LRCSPKSGHDTVSWYQQALGQGPQFIFQYYEEEERQR | |
| | GNFPDRFSGHQFPNYSSELNVNALLLGDSALYLCASS | |
| | LGWRGGRYNEQFFGPGTRLTVLEDLRNVTPPKVSLFE | |
| | PSKAEIANKQKATLVCLARGFFPDHVELSWWVNGKEV | |
| | HSGVCTDPQAYKESNYSYCLSSRLRVSATFWHNPRNH | |
| | FRCQVQFHGLSEEDKWPEGSPKPVTQNISAEAWGRAD | |
| | CGITSASYQQGVLSATILYEILLGKATLYAVLVSTLV | |
| | VMAMVKRKNSRAKRSGSGATNFSLLKQAGDVEENPGP | |
| | MWGVFLLYVSMKMGGTTGQNIDQPTEMTATEGAIVQI | |
| | NCTYQTSGFNGLFWYQQHAGEAPTFLSYNVLDGLEEK | |
| | GRFSSFLSRSKGYSYLLLKELQMKDSASYLCASVDGN | |
| | NRLAFGKGNQVVVIPNIQNPEPAVYQLKDPRSQDSTL | |
| | CLFTDFDSQINVPKTMESGTFITDKCVLDMKAMDSKS | |
| | NGAIAWSNQTSFTCQDIFKETNATYPSSDVPCDATLT | |
| | EKSFETDMNLNFQNLLVIVLRILLLKVAGFNLLMTLR | |
| | LWSS | |
| CER25_T2A_HPV16 E7 | MSKGLLLLWLVTELWWLYLTPAASEDTIIGFLGQPVT | SEQ ID NO: 179 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLLWNKKRMRGPGKDPTRKCPDPRSASSPKQHPSE | |
| | SVYTALQRRETEVYACIENEDGSSPTAKQSPLSQERP | |
| | HRFEDDGELNLVYENLLEGGGEGRGSLLTCGDVEENP | |
| | GPMAPGLLCWALLCLLGAGLVDAGVTQSPTHLIKTRG | |
| | QQVTLRCSPKSGHDTVSWYQQALGQGPQFIFQYYEEE | |
| | ERQRGNFPDRFSGHQFPNYSSELNVNALLLGDSALYL | |
| | CASSLGWRGGRYNEQFFGPGTRLTVLEDLRNVTPPKV | |
| | SLFEPSKAEIANKQKATLVCLARGFFPDHVELSWWVN | |
| | GKEVHSGVCTDPQAYKESNYSYCLSSRLRVSATFWHN | |
| | PRNHFRCQVQFHGLSEEDKWPEGSPKPVTQNISAEAW | |
| | GRADCGITSASYQQGVLSATILYEILLGKATLYAVLV | |
| | STLVVMAMVKRKNSRAKRSGSGATNFSLLKQAGDVEE | |
| | NPGPMWGVFLLYVSMKMGGTTGQNIDQPTEMTATEGA | |
| | IVQINCTYQTSGFNGLFWYQQHAGEAPTFLSYNVLDG | |
| | LEEKGRFSSFLSRSKGYSYLLLKELQMKDSASYLCAS | |
| | VDGNNRLAFGKGNQVVVIPNIQNPEPAVYQLKDPRSQ | |
| | DSTLCLFTDFDSQINVPKTMESGTFITDKCVLDMKAM | |
| | DSKSNGAIAWSNQTSFTCQDIFKETNATYPSSDVPCD | |
| | ATLTEKSFETDMNLNFQNLLVIVLRILLLKVAGFNLL | |
| | MTLRLWSS | |
| CER27_T2A_HPV16 E7 | MSKGLLLLWLVTELWWLYLTPAASEDTIIGFLGQPVT | SEQ ID NO: 180 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLHRFHGLWYMKMMWAWLQAKRKPRKAPSRNICYD | |
| | AFVSYSERDAYWVENLMVQELENFNPPFKLCLHKRDF | |
| | IPGKWIIDNIIDSIEKSHKTVFVLSENFVKSEWCKYE | |
| | LDFSHFRLFDENNDAAILILLEPIEKKAIPQRFCKLR | |
| | KIMNTKTYLEWPMDEAQREGFWVNLRAAIKSLEGGGE | |
| | GRGSLLTCGDVEENPGPMAPGLLCWALLCLLGAGLVD | |
| | AGVTQSPTHLIKTRGQQVTLRCSPKSGHDTVSWYQQA | |
| | LGQGPQFIFQYYEEEERQRGNFPDRFSGHQFPNYSSE | |
| | LNVNALLLGDSALYLCASSLGWRGGRYNEQFFGPGTR | |
| | LTVLEDLRNVTPPKVSLFEPSKAEIANKQKATLVCLA | |
| | RGFFPDHVELSWWVNGKEVHSGVCTDPQAYKESNYSY | |
| | CLSSRLRVSATFWHNPRNHFRCQVQFHGLSEEDKWPE | |
| | GSPKPVTQNISAEAWGRADCGITSASYQQGVLSATIL | |
| | YEILLGKATLYAVLVSTLVVMAMVKRKNSRAKRSGSG | |
| | ATNFSLLKQAGDVEENPGPMWGVFLLYVSMKMGGTTG | |
| | QNIDQPTEMTATEGAIVQINCTYQTSGFNGLFWYQQH | |
| | AGEAPTFLSYNVLDGLEEKGRFSSFLSRSKGYSYLLL | |
| | KELQMKDSASYLCASVDGNNRLAFGKGNQVVVIPNIQ | |
| | NPEPAVYQLKDPRSQDSTLCLFTDFDSQINVPKTMES | |
| | GTFITDKCVLDMKAMDSKSNGAIAWSNQTSFTCQDIF | |
| | KETNATYPSSDVPCDATLIEKSFETDMNLNFQNLLVI | |
| | VLRILLLKVAGFNLLMTLRLWSS | |
| CER29_T2A_HPV16 E7 | MSKGLLLLWLVTELWWLYLTPAASEDTIIGFLGQPVT | SEQ ID NO: 181 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLMSLLNCENSCGSSQSESDCCVAMASSCSAVTKD | |
| | DSVGGTASTGNLSSSFMEEIQGYDVEFDPPLESKYEC | |
| | PICLMALREAVQTPCGHRFCKACIIKSIRDAGHKCPV | |
| | DNEILLENQLFPDNFAKREILSLMVKCPNEGCLHKME | |
| | LRHLEDHQAHCEFALMDCPQCQRPFQKFHINIHILKD | |
| | CPRRQVSCDNCAASMAFEDKEIHDQNCPLANVICEYC | |
| | NTILIREQMPNHYDLDCPTAPIPCTFSTFGCHEKMQR | |
| | NHLARHLQENTQSHMRMLALEGGGEGRGSLLTCGDVE | |
| | ENPGPMAPGLLCWALLCLLGAGLVDAGVTQSPTHLIK | |
| | TRGQQVTLRCSPKSGHDTVSWYQQALGQGPQFIFQYY | |
| | EEEERQRGNFPDRFSGHQFPNYSSELNVNALLLGDSA | |
| | LYLCASSLGWRGGRYNEQFFGPGTRLTVLEDLRNVTP | |
| | PKVSLFEPSKAEIANKQKATLVCLARGFFPDHVELSW | |
| | WVNGKEVHSGVCTDPQAYKESNYSYCLSSRLRVSATF | |
| | WHNPRNHFRCQVQFHGLSEEDKWPEGSPKPVTQNISA | |
| | EAWGRADCGITSASYQQGVLSATILYEILLGKATLYA | |
| | VLVSTLVVMAMVKRKNSRAKRSGSGATNFSLLKQAGD | |
| | VEENPGPMWGVFLLYVSMKMGGTTGQNIDQPIEMTAT | |
| | EGAIVQINCTYQTSGFNGLFWYQQHAGEAPTFLSYNV | |
| | LDGLEEKGRFSSFLSRSKGYSYLLLKELQMKDSASYL | |
| | CASVDGNNRLAFGKGNQVVVIPNIQNPEPAVYQLKDP | |
| | RSQDSTLCLFTDFDSQINVPKTMESGTFITDKCVLDM | |
| | KAMDSKSNGAIAWSNQTSFTCQDIFKETNATYPSSDV | |
| | PCDATLIEKSFETDMNLNFQNLLVIVLRILLLKVAGF | |
| | NLLMTLRLWSS | |
| CER31_T2A_HPV16 E7 | MSKGLLLLWLVTELWWLYLTPAASEDTIIGFLGQPVT | SEQ ID NO: 182 |
| TCR | LPCHYLSWSQSRNSMCWGKGSCPNSKCNAELLRTDGT | |
| | RIISRKSTKYTLLGKVQFGEVSLTISNTNRGDSGVYC | |
| | CRIEVPGWFNDVKKNVRLELRRATTTKKPTTTTRPTT | |
| | TPYVTTTTPELLPTTVMTTSVLPTTTPPQTLATTAFS | |
| | TAVTTCPSTTPGSFSQETTKGSAFTTESETLPASNHS | |
| | QRSMMTISTDIAVLRPTGSNPGILPSTSQLTTQKTTL | |
| | TTSESLQKTTKSHQINSRQTILIIACCVGFVLMVLLF | |
| | LAFLMESSKKMDSPGALQTNPPLKLHTDRSAGTPVFV | |
| | PEQGGYKEKFVKTVEDKYKCEKCHLVLCSPKQTECGH | |
| | RFCESCMAALLSSSSPKCTACQESIVKDKVFKDNCCK | |
| | REILALQIYCRNESRGCAEQLMLGHLLVHLKNDCHFE | |
| | ELPCVRPDCKEKVLRKDLRDHVEKACKYREATCSHCK | |
| | SQVPMIALQKHEDTDCPCVVVSCPHKCSVQTLLRSEL | |
| | SAHLSECVNAPSTCSFKRYGCVFQGTNQQIKAHEASS | |
| | AVQHVNLLKEWSNSLEKKVLEGGGEGRGSLLTCGDVE | |
| | ENPGPMAPGLLCWALLCLLGAGLVDAGVTQSPTHLIK | |
| | TRGQQVTLRCSPKSGHDTVSWYQQALGQGPQFIFQYY | |
| | EEEERQRGNFPDRFSGHQFPNYSSELNVNALLLGDSA | |
| | LYLCASSLGWRGGRYNEQFFGPGTRLTVLEDLRNVTP | |
| | PKVSLFEPSKAEIANKQKATLVCLARGFFPDHVELSW | |
| | WVNGKEVHSGVCTDPQAYKESNYSYCLSSRLRVSATF | |
| | WHNPRNHFRCQVQFHGLSEEDKWPEGSPKPVTQNISA | |
| | EAWGRADCGITSASYQQGVLSATILYEILLGKATLYA | |
| | VLVSTLVVMAMVKRKNSRAKRSGSGATNFSLLKQAGD | |
| | VEENPGPMWGVFLLYVSMKMGGTTGQNIDQPTEMTAT | |
| | EGAIVQINCTYQTSGFNGLFWYQQHAGEAPTFLSYNV | |
| | LDGLEEKGRFSSFLSRSKGYSYLLLKELQMKDSASYL | |
| | CASVDGNNRLAFGKGNQVVVIPNIQNPEPAVYQLKDP | |
| | RSQDSTLCLFTDFDSQINVPKTMESGTFITDKCVLDM | |
| | KAMDSKSNGAIAWSNQTSFTCQDIFKETNATYPSSDV | |
| | PCDATLTEKSFETDMNLNFQNLLVIVLRILLLKVAGF | |
| | NLLMTLRLWSS | |

A selected CER polynucleotide and the HPV16 E7 TCR polynucleotide were inserted into the same pLenti lentiviral vector with a T2A sequence (encoding an amino acid sequence of SEQ ID NO:68) therebetween. (see, FIGS. 1A-1G). Peripheral blood was collected by venipuncture from a human donor, and human peripheral blood mononuclear cells (PBMCs) were isolated by density gradient centrifugation using lymphocyte separation media. CD8+ T cells were enriched from PBMCs using a commercially available isolation kit and activated with anti-CD3 and anti-CD28 in

Complete Cell Growth Media. 50 μl of viral vector expressing the CER-HPV16 E7 TCR combination were diluted in 0.5 ml Complete Cell Growth Media and added to the CD8+ T cells. The transduced CD8+ T cells were then centrifuged at 270 x g rpm for 1 hour in a 32° C. pre-warmed centrifuge. The CD8+ T cells were incubated for 24 hours at 37° C. T cells were expanded for another 72 hours in Complete Cell Growth Media, de-beaded, and allowed to expand for 5 days prior to being utilized for functional assays.

### Example 2: CD8 T Cells Transduced with Cer-TRC Tandem Expression Cassette Exhibit Antigen Specific Cytolytic and Phagocytic Activity

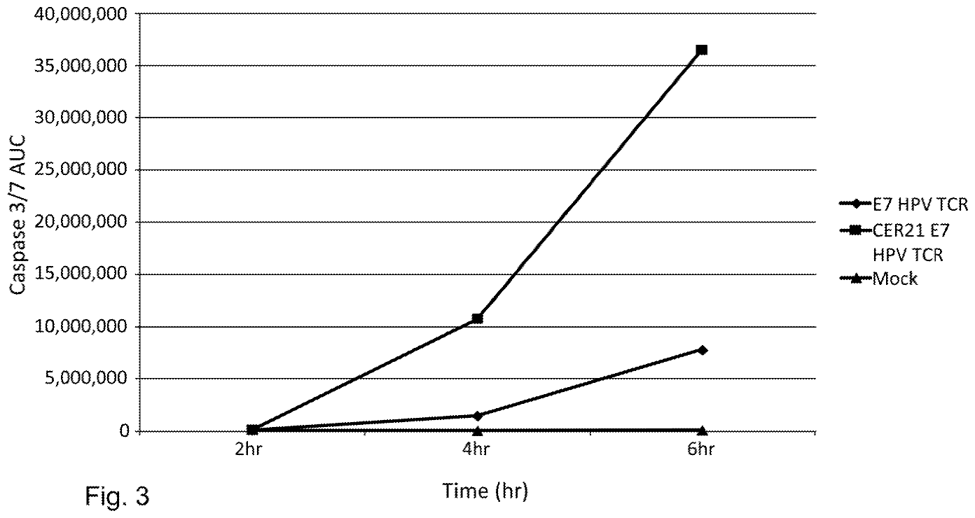

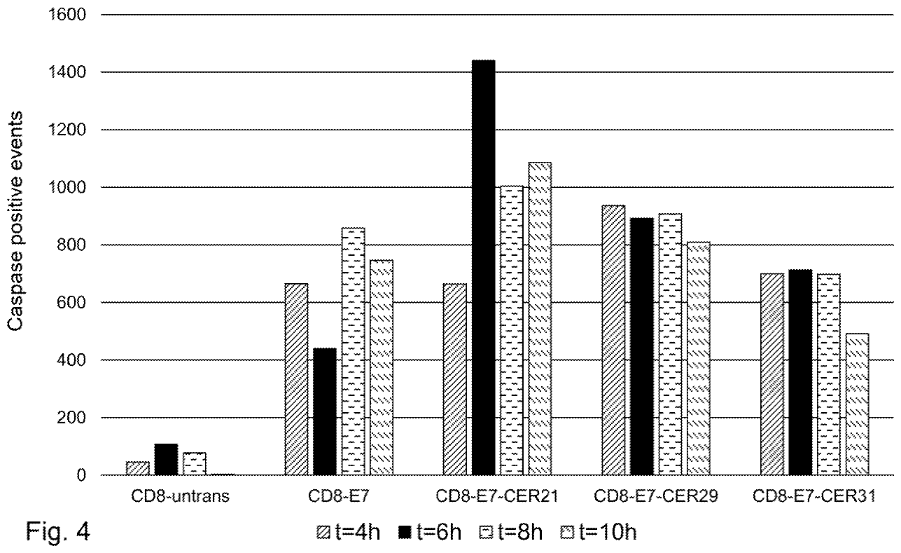

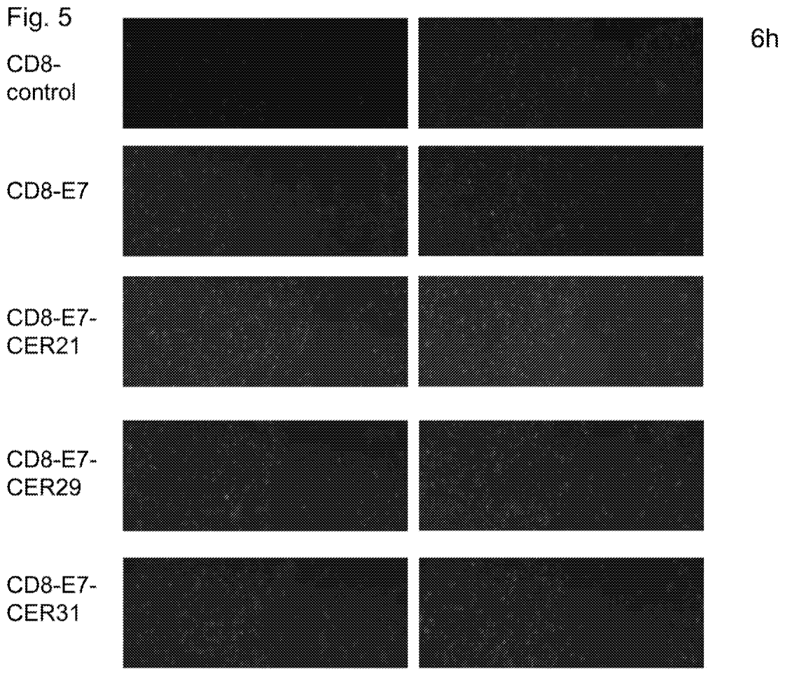

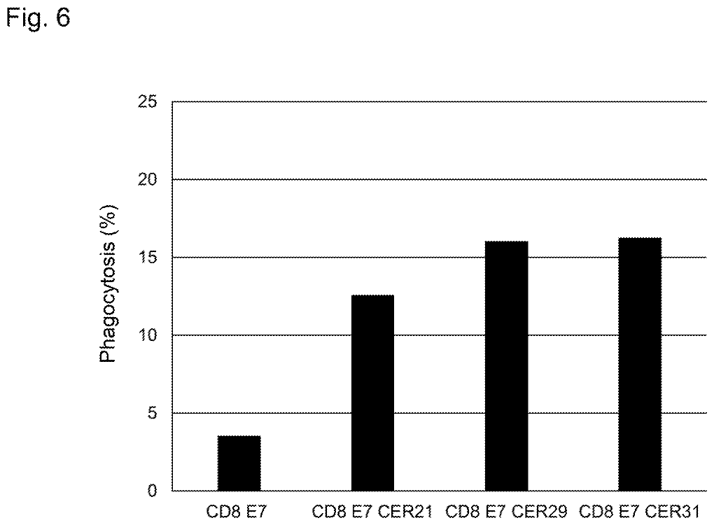

Cytotoxic activity of tandem expression cassette transduced CD8+ T cells was detected using a caspase 3/7 apoptosis reagent (IncuCyte®) that couples the activated caspase 3/7 recognition motif with a red reagent that fluoresces upon cleavage. The fluorescent signal was measured using fluorescent microscopy. Transduced CD8+ T cells were co-cultured with HPV16 E7+ head and neck squamous cell carcinoma cells (SCC152) at a 1:1 ratio, and caspase 3/7 apoptosis reagent was added to the co-culture. CD8+ T cells comprising CER21-HPV16 E7 TCR tandem expression cassette exhibit cytotoxic activity toward SCC152 cells (see, FIG. 2). The cytotoxic response by the CD8+ T cells transduced with CER21-HPV16 E7 TCR tandem expression cassette appears to be exponentially higher than the CD8+ T cells comprising HPV16 E7 TCR alone by 6 hours (see, FIG. 3). CD8+ T cells transduced with CER21-HPV16 E7 TCR tandem expression cassette, CER29-HPV16 E7 TCR tandem expression cassette, or CER31-HPV16 E7 TCR tandem expression cassette were co-cultured with SCC152 cells at a target:effector cell ratio of 1:1. The caspase 3/7 apoptosis reagent was added to the co-culture, and cytotoxic activity was measured over time by measuring fluorescence (see, FIGS. 4 and 5). Control samples were CD8+ T cells transduced with HPV16 E7 TCR alone or mock transduced T cells.

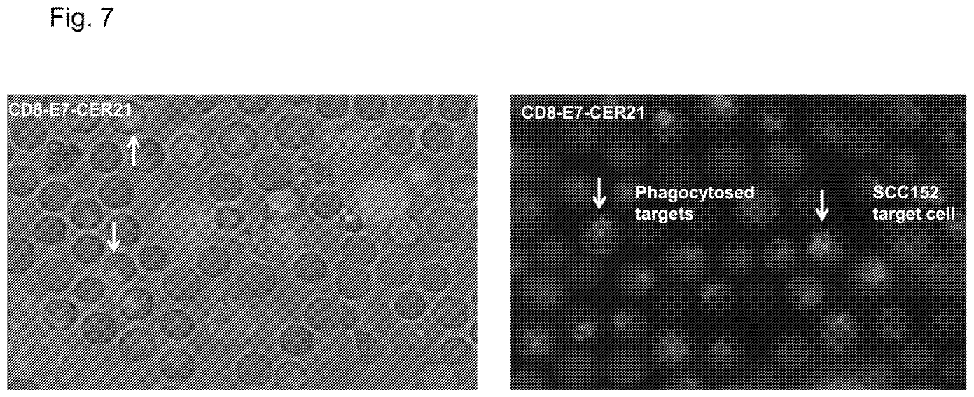

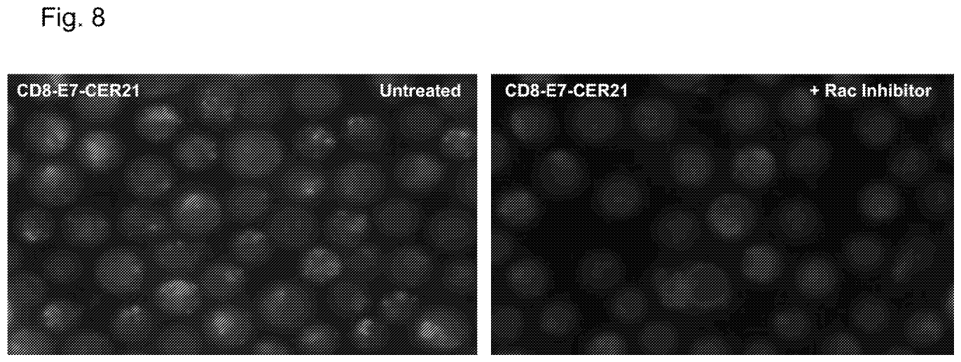

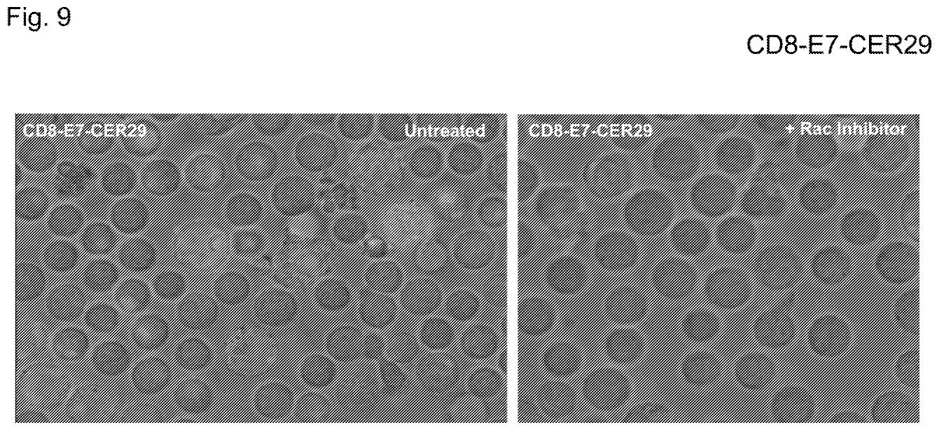

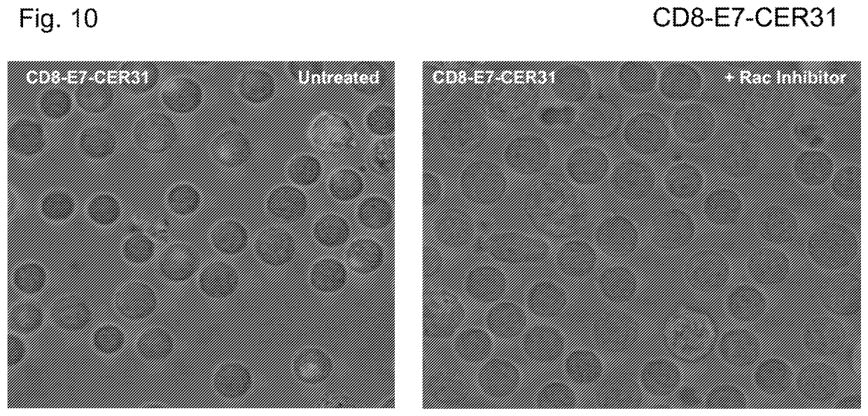

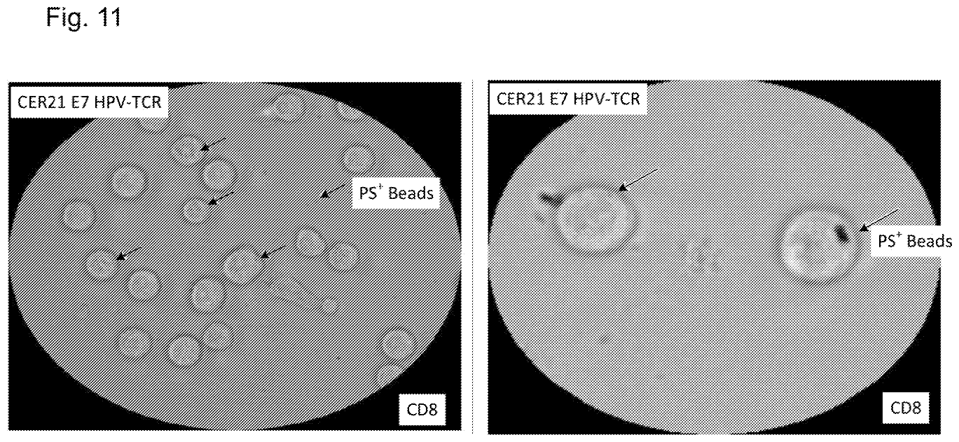

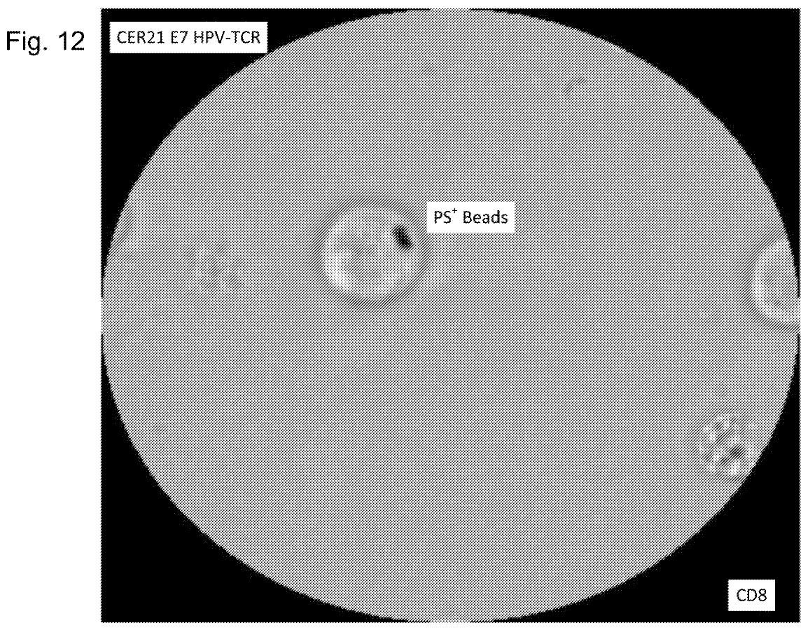

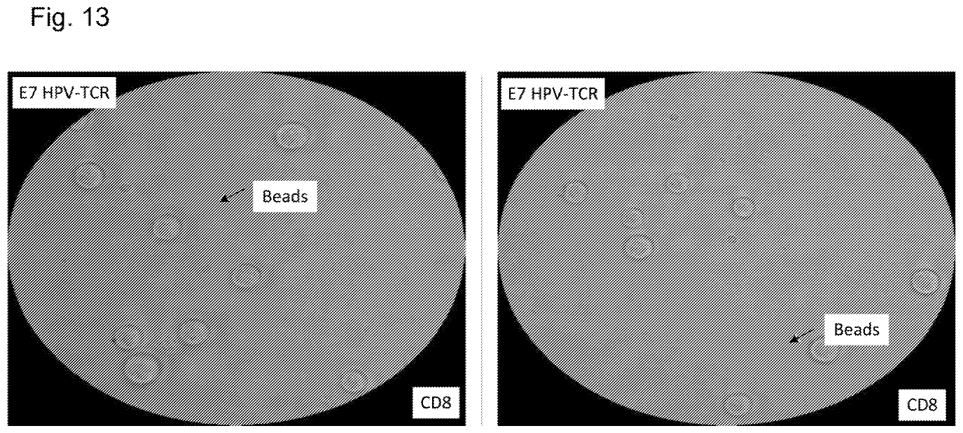

Phagocytic activity of tandem expression cassette transduced CD8+ T cells was detected by co-culturing tandem expression cassette transduced CD8+ T cells with SCC152 cells for 6 hours at a 1:1 ratio. Phagocytic events were visualized and quantified using KEYENCE BZ-X710 fluorescence microscope, 20× objective and hybrid capture software. CD8+ T cells transduced with CER21-HPV16 E7 TCR, CER29-HPV16 E7 TCR, or CER31-HPV16 E7 TCR tandem cassettes were capable of phagocytosing SCC152 cells (see, FIGS. 6 and 7). Rac1 inhibitor NSC23766 (50 μM) was also added to co-culture experiments and in vitro phagocytosis was measured. Treatment with Rac1 inhibitor revealed that the engulfment of SCC152 cells by the CER21-HPV16 E7 TCR, CER29-HPV16 E7 TCR, or CER31-HPV16 E7 TCR transduced T cells occurred in a Rac1-dependent manner (see, FIGS. 8-10). FIGS. 11 and 12 show that CD8+ T cells transduced with CER21-HPV16 E7 TCR tandem expression cassette engulfed strepavidin coated latex beads, to which were coated with biotin-conjugated phosphatidylserine. After about 30 minutes of incubation, the phosphatidylserine coated beads could be visualized inside the CER21-HPV16 E7 TCR+ T cells.

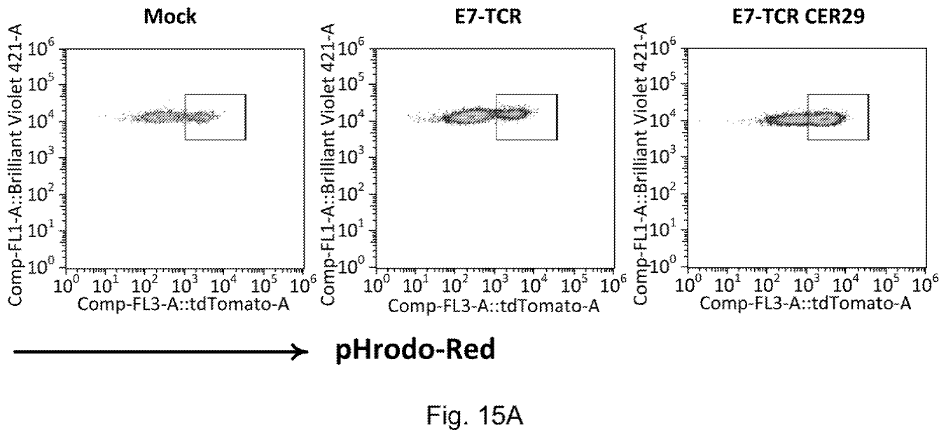

Cytokine response of CD8+ T cells transduced with CER21-HPV16 E7 TCR tandem expression cassette was measured during co-culture experiments with SCC152 cells by sampling the cellular supernatants and showed that CER21-HPV16 E7 TCR+ T cells exhibit antigen specific effector function as measured by IFNy response (see, FIG. 14).

### Example 3: Phagocytic Activity of T Cells Specifically Induced by CER

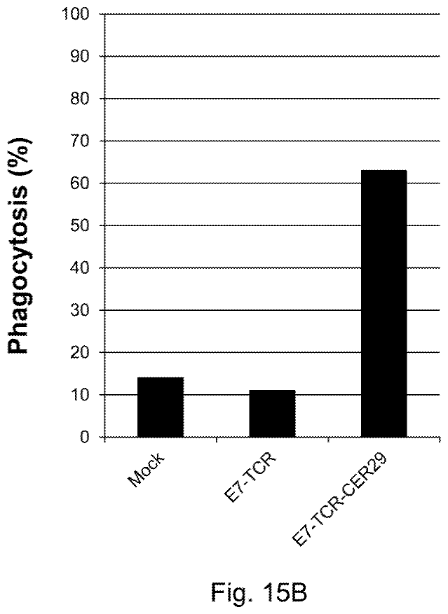

The ability of CER modified-T cells to engulf target cells and recapitulate engulfment-signaling events was evaluated in co-culture assays. T cells were transduced with a nucleic acid encoding HPV E7 specific TCR (polypeptide sequence comprising SEQ ID NO:158) or tandem expression cassette encoding HPV E7 specific TCR+ CER29 (polypeptide sequence comprising SEQ ID NO:169). pHrodo-labeled HPV+ SCC152 head and neck cancer cells were co-incubated with mock transduced T cells, HPV E7 TCR transduced T cells, or HPV E7 TCR/CER29-transduced T cells. Phagocytosis was analyzed by FACS, which showed large populations of cell-trace violet-labeled E7 TCR/CER29-T cells doubly stained with pHrodo positive (see FIG. 15A). Quantification of FACS data showed no difference between mock-transduced T cells and E7 TCR-transduced T cells (see FIG. 15B).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet including but not limited to U.S. Provisional Patent Application No. 62/649,499, filed Mar. 28, 2018, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An expression cassette comprising:
(a) a polynucleotide encoding a chimeric engulfment receptor (CER) comprising:
an extracellular domain comprising a binding domain that binds to a first target antigen;
an engulfment signaling domain;
a transmembrane domain positioned between and connecting the extracellular domain and the engulfment signaling domain; and
(b) a polynucleotide encoding a chimeric antigen receptor (CAR) comprising:
an extracellular domain comprising a binding domain that binds to a second target antigen;
an intracellular signaling domain; and
a transmembrane domain positioned between and connecting the extracellular domain and the intracellular signaling domain.

2. An expression cassette comprising:
(a) a polynucleotide encoding a chimeric engulfment receptor (CER) comprising:
an extracellular domain comprising a binding domain that binds to a first target antigen;
an engulfment signaling domain;
a transmembrane domain positioned between and connecting the extracellular domain and the engulfment signaling domain;
(b) a polynucleotide encoding a recombinant T cell receptor (TCR) binding protein beta chain comprising a TCR beta variable region and a TCR beta constant region; and
(c) a polynucleotide encoding a recombinant TCR alpha chain comprising a TCR alpha variable region and a TCR alpha constant region.

3. The expression cassette of claim 1 or 2, wherein the CER binding domain comprises a scFv, receptor ectodomain, or a ligand.

4. The expression cassette of any one of claims 1-3, wherein the CER extracellular domain further comprises a spacer domain.

5. The expression cassette of any one of claims 1-4, wherein the CER transmembrane domain comprises a Tim1, Tim4, Tim3, FcR, CD8, CD28, MERTK, Axl, Tyro3, BAI1, CD4, DAP12, MRC1, FcR, TLR1, TLR2, TLR3, TLR4, TLR5, TLR6, TLR7, TLR8, or TLR9 transmembrane domain.

6. The expression cassette of any one of claims 1-5, wherein the CER engulfment signaling domain comprises a MERTK, Tyro3, ItgB5, MRC1, BAI1, ELMO, Axl, Syk, MyD88, Zap70, FcγR1, FcγR2A, FcγR2B2, FcγR2C, FcγR3A, FcεR1, FcαR1, BAFF-R, DAP12, NFAM1, CD79b, TLR1, TLR2, TLR3, TLR4, TLR5, TLR6, TLR7, TLR8, TLR9, Traf6, Traf2, or Traf3 signaling domain.

7. The expression cassette of any one of claims 1-6, wherein the CER engulfment signaling domain comprises a primary engulfment signaling domain and a secondary engulfment signaling domain.

8. The expression cassette of any one of claims 1-7, wherein the CER primary and secondary engulfment signaling domains are different.

9. The expression cassette of any one of claims 1-8, wherein the CER primary and secondary engulfment signaling domains are each independently selected from MERTK, Tyro3, ItgB5, MRC1, BAI1, ELMO, Axl, Syk, MyD88, Zap70, FcγR1, FcγR2A, FcγR2B2, FcγR2C, FcγR3A, FcεR1, FcαRl, BAFF-R, DAP12, NFAM1, CD79b, TLR1, TLR2, TLR3, TLR4, TLR5, TLR6, TLR7, TLR8, TLR9, Traf6, Traf2, and Traf3 signaling domain.

10. The expression cassette of any one of claims 1-9, wherein the first target antigen of the CER is a pro-engulfment marker, tumor antigen, viral antigen, or parasite antigen.

11. The expression cassette of claim 10, wherein the pro-engulfment marker is phosphatidylserine.

12. The expression cassette of claim 11, wherein the CER binding domain comprises a Tim4 binding domain that binds phosphatidylserine.

13. The expression cassette of any one of claims 1 and 3-12, wherein the CAR binding domain comprises a scFv.

14. The expression cassette of any one of claims 1 and 3-13, wherein the CAR extracellular domain further comprises a spacer domain.

15. The expression cassette of any one of claims 1 and 3-14, wherein the CAR transmembrane domain comprises a CD28, CD2, CD4, CD8, CD3c, CD3δ, CD3ζ, CD25, CD27, CD40, CD79A, CD79B, CD80, CD86, CD95 (Fas), CD134 (OX40), CD137 (4-1BB), CD150 (SLAMF1), CD152 (CTLA4), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GALS, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, or Zap70 transmembrane domain.

16. The expression cassette of any one of claims 1 and 3-15, wherein the CAR intracellular signaling domain comprises an ITAM-containing activating signaling domain selected from CD3ζ, CD3γ, CD3δ, CD3ε, CD5, CD22, CD79a, CD278 (ICOS), DAP10, DAP12, CD79b, FcR, and CD66d signaling domains.

17. The expression cassette of any one of claims 1 and 3-16, wherein the CAR intracellular signaling domain comprises a first costimulatory signaling domain selected from CD27, CD28, 4-1BB, OX40, CD30, CD40, PD-1, ICOS, lymphocyte function-associated antigen-1 (LFA-1), CD2, CD7, LIGHT, NKG2C, and B7-H3 signaling domain.

18. The expression cassette of any one of claims 1 and 3-17, wherein the CAR intracellular signaling domain comprises a second costimulatory signaling domain selected from CD27, CD28, 4-1BB, OX40, CD30, CD40, PD-1, ICOS, lymphocyte function-associated antigen-1 (LFA-1), CD2, CD7, LIGHT, NKG2C, and B7-H3 signaling domain.

19. The expression cassette of any one of claims 1 and 3-18, wherein the CAR is a first generation CAR, second generation CAR, third generation CAR, or TCR-CAR.

20. The expression cassette of any one of claims 1 and 3-19, wherein the second target antigen of the CAR is a tumor antigen, viral antigen, or parasite antigen.

21. The expression cassette of any one of claims 20, wherein the second target antigen of the CAR is a tumor antigen selected from CD138, CD38, CD33, CD123, CD72, CD79a, CD79b, mesothelin, PSMA, BCMA, ROR1, MUC-16, L1CAM, CD22, CD19, CD20, CD23, CD24, CD37, CD30, CA125, CD56, c-Met, EGFR, GD-3, HPV E6, HPV E7, MUC-1, HER2, folate receptor a, CD97, CD171, CD179a, CD44v6, WT1, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, or GD2.

22. The expression cassette of any one of claims 2-12, wherein the second target antigen of the recombinant TCR is WT-1, mesothelin, MART-1, NY-ESO-1, MAGE-A3, HPV E7, survivin, a Fetoprotein, or a tumor neoantigen.

23. The expression cassette of any one of claims 2-12 and 22, wherein the TCR alpha chain comprises an LVL substitution at positions 12, 14, and 15.

24. The expression cassette of any one of claims 2-12, 22, and 23, wherein the TCR beta chain constant region comprises a cysteine substitution at position 56, TCR alpha chain constant region comprises a cysteine substitution at position 48, or both.

25. The expression cassette of any one of claims 1-24, wherein the polynucleotide encoding the CER is upstream of the polynucleotide encoding the CAR or TCR.

26. The expression cassette of any one of claims 1, 3-21, and 25, further comprising a polynucleotide encoding an IRES element or 2A peptide between the polynucleotide encoding the CER and the polynucleotide encoding the CAR.

27. The expression cassette of any one of claims 2-12, 22-24, and 25, wherein the polynucleotides encoding the CER, TCR alpha chain, and TCR beta chain are separated from each other by a first polynucleotide encoding IRES element or 2A peptide and a second polynucleotide encoding a IRES element or 2A peptide.

28. The expression cassette of any one of claim 26 or 27, wherein the 2A peptide comprises a T2A, P2A, E2A, or F2A peptide.

29. The expression cassette of claim 2, wherein:
(a) the CER comprises an amino acid sequence of any one of the SEQ ID NOs listed in Table 1; and
(b) the TCR comprises an amino acid sequence of SEQ ID NO:90.

30. The expression cassette of any one of claims 1-29, further comprising a promoter operably linked to the expression cassette.

31. A vector comprising the expression cassette of any one of claims 1-30.

32. The vector of claim 31, wherein the vector is a viral vector.

33. The vector of claim 32, wherein the viral vector is a retroviral vector or a lentiviral vector.

34. A host cell comprising the vector of any one of claims 31-33.

35. The host cell of claim 34, wherein the host cell is a T cell.

36. The host cell of claim 35, wherein the T cell is a CD4 T cell, CD8 T cell, or both.

37. The host cell of claim 35 or 36, wherein the T cell is a naïve T cell, central memory T cell, effector T cell, or any combination thereof

38. The host cell of any one of claims 34-37, wherein the host cell is human.

39. The host cell of any one of claims 34-38, wherein the host cell exhibits cytolytic activity towards a cell expressing the antigen targeted by the CAR or TCR and phagocytic activity towards a cell expressing the antigen targeted by the CER.

40. A method of treating a disease in a subject comprising administering an effective amount of the host cell of any one of claims 34-39.

41. The method of claim 40, wherein the disease is a cancer, viral infection, bacterial infection, or parasitic infection.

42. The method of claim 41, wherein the cancer is a solid tumor, melanoma, non-small cell lung cancer, renal cell carcinoma, renal cancer, a hematological cancer, prostate cancer, castration-resistant prostate cancer, colon cancer, rectal cancer, gastric cancer, esophageal cancer, bladder cancer, head and neck cancer, thyroid cancer, breast cancer, triple-negative breast cancer, ovarian cancer, cervical cancer, lung cancer, urothelial cancer, pancreatic cancer, glioblastoma, hepatocellular cancer, myeloma, multiple myeloma, leukemia, Hodgkin's lymphoma, non-Hodgkin's lymphoma, myelodysplastic syndrome, brain cancer, CNS cancer, or malignant glioma.

43. The method of any one of claims 39-42, wherein the host cell is autologous or allogeneic to the subject.

44. The method of any one of claims 39-43, wherein the host cell is administered to the subject in combination with an additional therapeutic agent.

45. The method of claim 44, wherein the additional therapeutic agent is an antibody, radiation therapy, chemotherapeutic agent, small molecule therapy, cellular immunotherapy, oncolytic virus, electropulse therapy, UV light therapy, high frequency ultrasound therapy, antibiotic, anti-fungal agent, or anti-viral agent.

46. The method of claim 44 or 45, wherein the additional therapeutic agent is administered at a subtherapeutic dose.
